SEQUENCE LISTING (12 sequences)

SEQ ID NO: 1 - DNA, 20 bp, Artificial
  Primer
aaggcctaatgccgaacaca

SEQ ID NO: 2 - DNA, 20 bp, Artificial
  Primer
gtctggcccttcttcacctc

SEQ ID NO: 3 - DNA, 21 bp, Artificial
  Primer
gtttatccgtggtgtgtctgg

SEQ ID NO: 4 - DNA, 20 bp, Artificial
  Primer
ctgagctatagaggcctggg

SEQ ID NO: 5 - DNA, 20 bp, Artificial
  Primer
cggaaagcacccgtatggag

SEQ ID NO: 6 - DNA, 20 bp, Artificial
  Primer
cgtccaccacgcctacatag

SEQ ID NO: 7 - DNA, 20 bp, Artificial
  Primer
aatggatgtgtgcagctcag

SEQ ID NO: 8 - DNA, 20 bp, Artificial
  Primer
ggggttgctcaaacactcat

SEQ ID NO: 9 - DNA, 20 bp, Artificial
  Primer
cctgcaccaccaactgctta

SEQ ID NO: 10 - DNA, 21 bp, Artificial
  Primer
ggccatccacagtcttctgag

SEQ ID NO: 11 - DNA, 22 bp, Artificial
  Primer
cgaggaatttgccaagctctga

SEQ ID NO: 12 - DNA, 24 bp, Artificial
  Primer
ttcgggcactgcaggaacaaattc

(19) United States
(12) Patent Application Publication
(10) Pub. No.: US 2022/0041991 A1
(43) Pub. Date: Feb. 10, 2022

(54) CELL STRUCTURE INCLUDING SMALL INTESTINAL EPITHELIAL CELLS, METHOD FOR PRODUCING SAME, AND SUBSTRATE HOLDING SAME

(71) Applicants: DAI NIPPON PRINTING CO., LTD., Tokyo (JP); NATIONAL CENTER FOR CHILD HEALTH AND DEVELOPMENT, Tokyo (JP)

(72) Inventors: Yuichi TANAKA, Tokyo (JP); Koji FUJIMOTO, Tokyo (JP); Natsuki KAKEGAWA, Tokyo (JP); Hidenori AKUTSU, Tokyo (JP)

(73) Assignees: DAI NIPPON PRINTING CO., LTD., Tokyo (JP); NATIONAL CENTER FOR CHILD HEALTH AND DEVELOPMENT, Tokyo (JP)

(21) Appl. No.: 17/426,380

(22) PCT Filed: Jan. 29, 2020
(86) PCT No.: PCT/JP2020/003258

§ 371 Date: Jul. 28, 2021

(30) Foreign Application Priority Data
Jan. 30, 2019 (JP) 2019-014766

(51) Int. Cl. C12N 5/071; C12N 5/00

(52) U.S. Cl. CPC C12N 5/0679; C12N 2535/10; C12N 2533/30; C12N 5/0068

(57) ABSTRACT

The method for producing a cell construct including small intestinal epithelial cells includes: seeding stem cells onto cell culture substrate, the cell culture substrate having surface including cell culture part, wherein the cell culture part includes non-cell-adhesive part, and cell-adhesive part extending continuously or intermittently along periphery of the non-cell-adhesive part and surrounding the non-cell-adhesive part; and culturing the stem cells seeded to differentiate a part of the stem cells into small intestinal epithelial cells.

### TECHNICAL FIELD

The present disclosure relates to a cell construct comprising small intestinal epithelial cells, and a method for producing the cell construct comprising small intestinal epithelial cells.

The present disclosure also relates to a substrate holding the cell construct comprising small intestinal epithelial cells.

### BACKGROUND ART

As pluripotent stem cells such as embryonic stem cells (ES cells) and induced pluripotent stem cells (iPS cells) can be induced to differentiate into cells of interest, clinical applications of pluripotent stem cells in the field of regenerative medicine are expected. Methods for obtaining a structure called organoid similar to human tissues by culturing pluripotent stem cells to induce their differentiation are under study.

For example, Patent Literature 1 describes a method for producing an intestinal epithelial organoid from embryonic stem cells.

As disclosed in Non Patent Literature 2, the technique of inducing non-epithelial cells from an organoid by administering TNF-α into a medium has also been established.

Other examples of the method for inducing the differentiation of induced pluripotent stem cells into small intestinal epithelial cells include methods described in Patent Literature 2.

Patent Literature 3 and Non Patent Literature 3 each describe a method for inducing differentiation into an intestinal tissue by culturing pluripotent stem cells on a substrate on which a pattern of a cell-adhesive part is formed.

### CITATION LIST

### Patent Literature

Patent Literature 1: WO2011/140441
Patent Literature 2: JP Patent No. 6296399
Patent Literature 3: JP Patent No. 6151097
Patent Literature 4: JP Patent Publication (Kokai) No. 2012-120443 A (2012)
Patent Literature 5: JP Patent Publication (Kokai) No. 2013-179910 A (2013)
Patent Literature 6: JP Patent No. 5070565

### Non Patent Literature

Non Patent Literature 1: Workman et al., Nature Medicine, Vol. 23, p. 49-59, 2017
Non Patent Literature 2: Hahn et al., Scientific Reports, Vol. 7, 2435, 2017
Non Patent Literature 3: Uchida et al., JCI Insight, Vol. 2, e86492, 2017
Non Patent Literature 4: Lee et al., Stem Cell Reports, Vol. 6, p. 257-272, 2016
Non Patent Literature 5: Okeyo et al., Tissue Eng Part C, Vol. 21, p. 1105-1115, 2015
Non Patent Literature 6: Golos et al., Placenta, Vol. 34S, p. S56-S61, 2013
Non Patent Literature 7: Chen et al., Biochemical and Biophysical Research Communications, Vol. 436, p. 677-684, 2013
Non Patent Literature 8: Kojima et al., Laboratory Investigation, Vol. 97, p. 1188-1200, 2017
Non Patent Literature 9: Niwa et al., Nature Genetics, Vol. 24, p. 372-376, 2000
Non Patent Literature 10: Onozato et al., Drug and Metabolism Disposition Epub ahead of print, 2018
Non Patent Literature 11: Tsunoda et al., The Journal of Japan Society for Laser Surgery and Medicine, Vol. 28, p. 355-361, 2008
Non Patent Literature 12: Mao et al., Pharmaceutical Research, Vol. 25, p. 1244-1255, 2008
Non Patent Literature 13: Mustata et al., Cell Reports, Vol. 5, p. 421-432, 2013
Non Patent Literature 14: Boyd, Placenta, Vol. 27, p. S24-S26, 2013
Non Patent Literature 15: Han et al., Expert Opinion on Drug Metabolism & Toxicology, Vol. 14, p. 817-829, 2018
Non Patent Literature 16: Kabeya et al., Drug Metabolism and Disposition, Vol. 46, p. 1411-1419, 2018
Non Patent Literature 17: Singh et al., Cell Stem Cell, Vol. 10, p. 312-326, 2012
Non Patent Literature 18: Akutsu et al., Regenerative Therapy, Vol. 1, p. 18-29, 2015
Non Patent Literature 19: Okochi et al., Langmuir, Vol. 25, p. 6947-6953, 2009

### SUMMARY OF INVENTION

### Technical Problem

The intestine is a complex organ including cells derived from all 3 germ layers (endoderm, ectoderm, and mesoderm). The intestine, which is composed of endoderm-derived small intestinal epithelial cells (e.g., intestine cells, goblet cells, endocrine cells, brush cells, Paneth cells, and M cells), mesoderm-derived lymph tissue, smooth muscle cells, interstitial cells of Cajal, ectoderm-derived intestinal nerve plexus, and other types of cells in a complex manner, has a variety of functions including secretion, absorption, and peristaltic movement.

A tissue obtained by the method described in Patent Literature 1 contains only small intestinal epithelial cells. Since activin is used in the induction of differentiation of embryonic stem cells, only cells derived from a substantially single germ layer, i.e., endoderm-derived cells therein, are formed. Hence, for obtaining intestine tissues containing other types of cells derived from mesoderm or ectoderm, it is necessary to separately induce differentiation into these other types of cells, as disclosed in Non Patent Literature 1. The method described in Patent Literature 1 comprises the step of culturing cells embedded in Matrigel for the induction of epithelial differentiation and thus has problems associated with productivity in this respect.

The method described in Non Patent Literature 2 disadvantageously requires labor for culture.

Since a tissue obtained by the method described in Patent Literature 2 contains only small intestinal epithelial cells, this method also has problems similar to those of the method described in Patent Literature 1.

Meanwhile, according to the method described in Patent Literature 3 and Non Patent Literature 3, differentiation not only into intestinal epithelial tissues but into muscular tissues, nervous tissues, and the like can be induced by single culture. This method has high production efficiency because many intestinal tissues can be cultured at the same time on one substrate on which many patterns are formed. The method is also easily applicable to transplantation because the culture is achieved without the use of organism-derived materials.

However, the method described in Patent Literature 3 and Non Patent Literature 3 is still susceptible to improvement for application to industrial production, which is required to have uniformity, in terms of a long culture period and low yields of intestine tissues.

Specifically, there has been a demand for producing a cell construct comprising small intestinal epithelial cells at high yields in a relatively short culture period.

Any cell construct comprising small intestinal epithelial cells has not been provided so far in a form suitable for use in tests aimed at the development of drugs for preventing or treating intestinal diseases, or pathological studies on intestinal diseases.

### Solution to Problem

The present disclosure encompasses the following aspects of the invention.

(1) A method for producing a cell construct comprising small intestinal epithelial cells, comprising:

seeding stem cells onto a cell culture substrate,

the cell culture substrate having a surface comprising a cell culture part, the cell culture part comprising a non-cell-adhesive part, and a cell-adhesive part extending continuously or intermittently along the periphery of the non-cell-adhesive part and surrounding the non-cell-adhesive part; and

culturing the stem cells seeded to differentiate a part of the stem cells into small intestinal epithelial cells.

(2) The method according to (1), wherein a distance between two points of intersection of a straight line with an inner circumference of the cell-adhesive part is larger than 80 μm and 880 μm or smaller, the straight line passing through a midpoint between two points on the inner circumference of the cell-adhesive part opposed to and most distant from each other across the non-cell-adhesive part.

(3) The method according to (1) or (2), wherein a width of the cell-adhesive part along a straight line is larger than 30 μm and 400 μm or smaller, the straight line passing through a midpoint between two points on an inner circumference of the cell-adhesive part opposed to and most distant from each other across the non-cell-adhesive part.

(4) The method according to any of (1) to (3), wherein ratio X′/W′ of distance X′ to width W′ is preferably 0.5 or more, more preferably 1.0 or more, and further preferably 1.3 or more, and is preferably 20.0 or less, more preferably 15.0 or less, and further preferably 10.0 or less,
wherein the distance X is a distance between two points of intersection of a straight line with an inner circumference of the cell-adhesive part, the straight line passing through a midpoint between two points on the inner circumference of the cell-adhesive part opposed to and most distant from each other across the non-cell-adhesive part, and
wherein the width W′ is a width of the cell-adhesive part along a straight line passing through the non-cell-adhesive part.

(5) The method according to (1), wherein a distance between two points of intersection of the periphery of the non-cell-adhesive part with a straight line passing through the barycenter of the non-cell-adhesive part is larger than 80 μm and 880 μm or smaller.

(6) The method according to (1) or (5), wherein a width of the cell-adhesive part along the straight line passing through the barycenter of the non-cell-adhesive part is larger than 30 μm and 400 μm or smaller.

(7) The method according to (1), (5) or (6), wherein ratio X/W of distance X to width W is preferably 0.5 or more, more preferably 1.0 or more, and further preferably 1.3 or more, and is preferably 20.0 or less, more preferably 15.0 or less, and further preferably 10.0 or less, wherein the distance X is a distance between two points of intersection of the periphery of the non-cell-adhesive part with a straight line passing through the barycenter of the non-cell-adhesive part, and
wherein the width W is a width of the cell-adhesive part along a straight line passing through the barycenter of the non-cell-adhesive part.

(8) A method for producing a cell construct comprising small intestinal epithelial cells, comprising:

seeding stem cells onto a cell culture substrate,

the cell culture substrate comprising a support substrate having a surface comprising

a first non-cell-adhesive part, and

one or more cell culture parts arranged in the first non-cell-adhesive part, wherein

each of the one or more cell culture parts comprises a central part serving as a second non-cell-adhesive part, and a cell-adhesive part extending continuously or intermittently along the periphery of the central part and surrounding the central part; and

culturing the stem cells seeded to differentiate a part of the stem cells into small intestinal epithelial cells.

(9) The method according to (8), wherein a distance between two points of intersection of a straight line with an inner circumference of the cell-adhesive part is larger than 80 μm and 880 μm or smaller, the straight line passing through a midpoint between two points on the inner circumference of the cell-adhesive part opposed to and most distant from each other across the central part .

(10) The method according to (8) or (9), wherein a width of the cell-adhesive part along a straight line is larger than 30 μm and 400 μm or smaller, the straight line passing through a midpoint between two points on an inner circumference of the cell-adhesive part opposed to and most distant from each other across the central part.

(11) The method according to any of (8) to (10), wherein ratio X′/W′ of distance X′ to width W′ is preferably 0.5 or more, more preferably 1.0 or more, and further preferably 1.3 or more, and is preferably 20.0 or less, more preferably 15.0 or less, and further preferably 10.0 or less,
wherein the distance X is a distance between two points of intersection of a straight line with an inner circumference of the cell-adhesive part, the straight line passing through a midpoint between two points on the inner circumference of the cell-adhesive part opposed to and most distant from each other across the central part, and
wherein the width W′ is a width of the cell-adhesive part along a straight line passing through the central part.

(12) The method according to (8), wherein a distance between two points of intersection of the periphery of the central part with a straight line passing through the barycenter of the central part is larger than 80 μm and 880 μm or smaller.

(13) The method according to (8) or (12), wherein a width of the cell-adhesive part along a straight line passing through the barycenter of the central part is larger than 30 μm and 400 μm or smaller.

(14) The method according to (8), (12) or (13), wherein ratio X/W of distance X to width W is preferably 0.5 or more, more preferably 1.0 or more, and further preferably 1.3 or more, and is preferably 20.0 or less, more preferably 15.0 or less, and further preferably 10.0 or less,
wherein the distance X is a distance between two points of intersection of the periphery of the central part with a straight line passing through the barycenter of the central part, and
wherein the width W is a width of the cell-adhesive part along a straight line passing through the barycenter of the central part.

(15) A cell construct comprising small intestinal epithelial cells, wherein

when an expression level of ABCG2 transporter and an expression level of ABCB1 transporter in the cell construct are indicated as relative values with respect to an expression level of ABCG2 transporter and an expression level of ABCB1 transporter, respectively, in a small intestinal tissue of an origin animal, the relative value of the expression level of ABCG2 transporter in the cell construct is 90 or more times the relative value of the expression level of ABCB1 transporter.

(16) A cell construct comprising small intestinal epithelial cells, wherein

when an expression level of CUBN and an expression level of villin in the cell construct are indicated as relative values with respect to an expression level of CUBN and an expression level of villin, respectively, in a small intestinal tissue of an origin animal, a ratio of the relative value of the expression level of CUBN to the relative value of the expression level of villin in the cell construct is in a range of 0.2 to 1.8.

(17) The cell construct according to (15) or (16), wherein each of the expression levels is based on a mRNA level.

(18) The cell construct according to any of (15) to (17), comprising endodermal cells, ectodermal cells and mesodermal cells.

(19) A cell construct-holding substrate comprising:

a cell culture substrate having a surface comprising a cell culture part, the cell culture part comprising a non-cell-adhesive part, and a cell-adhesive part extending continuously or intermittently along the periphery of the non-cell-adhesive part and surrounding the non-cell-adhesive part; and

a cell construct comprising small intestinal epithelial cells adhering onto the cell culture part.

(20) The cell construct-holding substrate according to (19), wherein when the cell construct-holding substrate is viewed along the normal direction of the surface, the small intestinal epithelial cells are present at least at a position overlapping the cell-adhesive part.

(21) The cell construct-holding substrate according to (20), wherein

when the cell construct-holding substrate is viewed along the normal direction of the surface, the cell construct overlaps the cell-adhesive part and the non-cell-adhesive part, and

the small intestinal epithelial cells are present in a larger amount at a position overlapping the cell-adhesive part than at a position overlapping the non-cell-adhesive part.

(22) The cell construct-holding substrate according to any of (19) to (21), wherein the cell construct comprises endodermal cells, ectodermal cells and mesodermal cells.

(23) The cell construct-holding substrate according to any of (19) to (22), wherein a distance between two points of intersection of a straight line with an inner circumference of the cell-adhesive part is larger than 80 μm and 880 μm or smaller, the straight line passing through a midpoint between two points on the inner circumference of the cell-adhesive part opposed to and most distant from each other across the non-cell-adhesive part.

(24) The cell construct-holding substrate according to any of (19) to (23), wherein a width of the cell-adhesive part along a straight line is larger than 30 μm and 400 μm or smaller, the straight line passing through a midpoint between two points on an inner circumference of the cell-adhesive part opposed to and most distant from each other across the non-cell-adhesive part.

(25) The cell construct-holding substrate according to any of (19) to (24), wherein ratio X′/W′ of distance X′ to width W′ is preferably 0.5 or more, more preferably 1.0 or more, and further preferably 1.3 or more, and is preferably 20.0 or less, more preferably 15.0 or less, and further preferably 10.0 or less,
wherein the distance X is a distance between two points of intersection of a straight line with an inner circumference of the cell-adhesive part, the straight line passing through a midpoint between two points on the inner circumference of the cell-adhesive part opposed to and most distant from each other across the non-cell-adhesive part, and
wherein the width W′ is a width of the cell-adhesive part along a straight line passing through the non-cell-adhesive part.

The present specification encompasses the contents disclosed in Japanese Patent Application No. 2019-014766 on which the priority of the present application is based.

### Advantageous Effects of Invention

The method of the present disclosure can produce a cell construct comprising small intestinal epithelial cells at high yields in a relatively short culture period.

The cell construct of the present disclosure can be used as a gut organoid in the development of drugs for preventing or treating intestinal diseases or pathological studies on intestinal diseases.

The cell construct-holding substrate of the present disclosure facilitates handling a cell construct comprising small intestinal epithelial cells for use in the development of drugs for preventing or treating intestinal diseases or pathological studies on intestinal diseases.

### BRIEF DESCRIPTION OF DRAWINGS

FIG. 1 is a schematic view of an embodiment in which a cell-adhesive part is an exposed surface of a support substrate, of a cell culture substrate having a plurality of annular cell-adhesive parts used in Example 1 and Example 8. FIG. 1(A) is a plane view of the cell culture substrate, and FIG. 1(B) is a cross-sectional schematic view taken along the A-A line in FIG. 1(A).

FIG. 2 shows observation images of cultures on culture days 1, 6, 11 and 18 obtained by culture using each cell culture substrate having an annular cell-adhesive part having a different inside diameter in Example 1.

FIG. 3 shows observation images of cultures on culture week 3 obtained by culture using each cell culture substrate having an annular cell-adhesive part having a different inside diameter in Example 1.

FIG. 4 shows observation images of a tissue having a sac-shaped structure formed on and detached from a substrate having an annular cell-adhesive part having an inside diameter of 380 μm (the left photograph shows the whole dish, and the right photograph is an observation image of the tissue).

FIG. 5 shows microscopic observation images of cultures on 3 weeks after the start of culture on a substrate in each of Comparative Example 1, Comparative Example 2, and Comparative Example 3.

FIG. 6 shows observation images of cells around one annular cell-adhesive part on each of culture days 4, 9, 13 and 20 in Example 2.

FIG. 7 shows observation images on culture days 1 and 7 of cultures obtained by culture using cell culture substrates differing in inside diameter in Example 3, and observation images of tissues having a sac-shaped structure collected after 3-week culture using a cell culture substrate having an annular cell-adhesive part having an inside diameter of 280 μm.

FIG. 8 shows observation images on culture day 4 of immunostained cultures in Example 4.

FIG. 9 shows observation images on culture day 7 of immunostained cultures in Example 4.

FIG. 10 shows results of staining, with an anti-CDX2 antibody, an anti-villin antibody and DAPI, of tissues formed by culturing stem cells on a cell culture substrate having an annular cell-adhesive part having an inside diameter of 280 μm or 380 μm and a width of 60 μm in Example 5.

FIG. 11 shows results of staining, with an anti-smooth muscle actin antibody, an anti-PGP9.5 antibody and DAPI, of tissues formed by culture on a cell culture substrate having an annular cell-adhesive part having an inside diameter of 380 μm and a width of 60 μm in Example 5.

FIG. 12 shows observation images on day 18 of culture on a cell culture substrate having an annular cell-adhesive part of each size in Example 6.

FIG. 13 shows results of evaluating culture on a cell culture substrate having an annular cell-adhesive part of each size in Example 6.

FIG. 14 shows an observation image of a tissue having a sac-shaped structure obtained by culture on a cell culture substrate having an annular cell-adhesive part having an inside diameter of 580 μm and a width of 60 μm in Example 6.

FIG. 15A shows a square cell-adhesive part having an inside dimension of 280 μm to 300 μm in side and a width of 50 μm to 60 μm used in Example 7.

FIG. 15B shows an annular cell-adhesive part having an inside diameter of 280 μm and a width of 60 μm and lacking ⅛ in the circumferential direction used in Example 7.

FIG. 15C shows a rectangular cell-adhesive part having an inside dimension of 600 μm in long side and 300 μm in short side and a width of 50 μm used in Example 7.

FIG. 15D shows a square cell-adhesive part having an inside dimension of 600 μm in side and a width of 50 μm used in Example 7.

FIG. 16A shows observation images of a culture obtained by culturing stem cells using a cell culture substrate having a cell-adhesive part in the shape shown in FIG. 15A.

FIG. 16B shows observation images of a culture obtained by culturing stem cells using a cell culture substrate having a cell-adhesive part in the shape shown in FIG. 15B.

FIG. 16C shows observation images of a culture obtained by culturing stem cells using a cell culture substrate having a cell-adhesive part in the shape shown in FIG. 15C.

FIG. 16D shows observation images of a culture obtained by culturing stem cells using a cell culture substrate having a cell-adhesive part in the shape shown in FIG. 15D.

FIG. 17 shows observation images on culture days 1, 7 and 11 of cultures obtained by culturing stem cells on a cell culture substrate having an annular cell-adhesive part having an inside diameter of 280 μm or 380 μm and a width of 60 μm in Example 8.

FIG. 18 shows observation images of tissues having a sac-shaped structure obtained by culturing stem cells for 3 weeks on a cell culture substrate having an annular cell-adhesive part having an inside diameter of 280 μm or 380 μm and a width of 60 μm in Example 8.

FIG. 19 shows an average ABCG2/ABCB1 ratio for a cell construct obtained on a cell culture substrate under each condition determined in Example 9.

FIG. 20 shows an average CUBN/villin ratio for a cell construct obtained on a cell culture substrate under each condition determined in Example 9.

FIG. 21 shows observation images of immunostaining of cells that adhered to a cell-adhesive part when Edom iPS stem cells were cultured on a cell culture substrate having a round cell-adhesive part having a diameter of 1500 μm in Example 10.

FIG. 22 shows observation images of immunostaining of cells that adhered onto a substrate when Edom iPS stem cells were cultured on each cell culture substrate, i.e., a dish for cell culture having a diameter of 3.5 cm (culture dish), a glass substrate without any pattern (both, 1.5 cm×2.5 cm in size) (blank glass), and a substrate having a round cell-adhesive part having a diameter of 1500 μm (patterned substrate) in Example 10.

FIG. 23 shows observation images of tissues on culture day 7 when Edom iPS cells were cultured on a substrate having a round cell-adhesive part having a diameter of 1500 μm.

FIG. 24 shows a relative value of the expression level of Oct3/4 in a sample of an accumulating portion with the expression level of Oct3/4 in a sample of a non-accumulating portion defined as 1 when the expression level of Oct3/4 was normalized with the expression level of GAPDH to determine a normalized value as to each of the samples of the accumulating portion and the non-accumulating portion in Example 11.

FIG. 25 shows results of immunostaining in Example 12.

FIG. 26 is a schematic view of an embodiment in which a cell-adhesive part is a surface of cell-adhesive layer, of a cell culture substrate having a plurality of annular cell-adhesive parts. FIG. 26(A) is a plane view of the cell culture substrate, and FIG. 26(B) is a cross-sectional schematic view taken along the A-A line in FIG. 26(A).

FIG. 27 is photographs showing results of immunostaining of nerve cells and muscle cells in a culture on culture day 9 when Edom iPS cells were cultured on a cell culture substrate having an annular cell-adhesive part having an inside diameter of 600 μm and a width of 100 μm in Example 4. The scale bar represents 100 μm.

FIG. 28 is photographs showing results of immunostaining of nerve cells and intestinal epithelial endodermal cells in a culture on culture day 9 when Edom iPS cells were cultured on a cell culture substrate having an annular cell-adhesive part having an inside diameter of 600 μm and a width of 100 μm in Example 4. The scale bar represents 100 μm.

FIG. 29 shows results of study using an accumulating portion after 1-week culture in Example 13. The scale bar represents 40 μm.

FIG. 30 shows results of study using an accumulating portion after 2-week culture in Example 13. The scale bar represents 40 μm.

FIG. 31 shows results of observation on culture day 4 after medium replacement in Example 14.

FIG. 32 shows results of observation on culture day 10 after medium replacement in Example 14.

FIG. 33 is a schematic view of an embodiment in which a cell construct is present at a position overlapping a cell-adhesive part, of a cell construct-holding substrate comprising a cell culture substrate having a non-cell-adhesive part (central part) and an annular cell-adhesive part surrounding it, and a cell construct comprising small intestinal epithelial cells. FIG. 33(A) is a plane view of the cell construct-holding substrate viewed along the normal direction of surface S. FIG. 33(B) is a cross-sectional schematic view taken along the A-A line in FIG. 33(A).

FIG. 34 is a schematic view of an embodiment in which a cell construct is present at a position overlapping a cell-adhesive part and a non-cell-adhesive part (central part), of a cell construct-holding substrate comprising a cell culture substrate having a non-cell-adhesive part (central part) and an annular cell-adhesive part surrounding it, and a cell construct comprising small intestinal epithelial cells. FIG. 34(A) is a plane view of the cell construct-holding substrate viewed along the normal direction of surface S. FIG. 34(B) is a cross-sectional schematic view taken along the A-A line in FIG. 34(A).

FIG. 35 is a schematic view of one embodiment of a cell culture substrate having a cell culture part comprising a non-cell-adhesive part (central part) and a cell-adhesive part surrounding it on each of the upper surfaces of a plurality of protrusions. FIG. 35(A) is a plane view of the cell culture substrate. FIG. 35(B) is a cross-sectional schematic view taken along the A-A line in FIG. 35(A).

FIG. 36 is a schematic view of one embodiment of a cell culture substrate having a cell culture part comprising a non-cell-adhesive part (central part) and a cell-adhesive part surrounding it on each of the bottom surfaces of a plurality of depressions. FIG. 36(A) is a plane view of the cell culture substrate. FIG. 36(B) is a cross-sectional schematic view taken along the A-A line in FIG. 36(A).

FIG. 37 is a schematic view of one embodiment of a cell culture substrate in which a cell culture part comprising a non-cell-adhesive part (central part) and a cell-adhesive part surrounding it occupies the whole of one surface. FIG. 37(A) is a plane view of the cell culture substrate. FIG. 37(B) is a cross-sectional schematic view taken along the A-A line in FIG. 37(A).

### DESCRIPTION OF EMBODIMENTS

### <1. Stem Cell>

Stem cells used in an embodiment of the present disclosure can be stem cells having the ability to differentiate into small intestinal epithelial cells and are preferably stem cells having the ability to differentiate into endodermal cells (small intestinal epithelial cells, etc.), ectodermal cells and mesodermal cells, and more preferably pluripotent stem cells. Particularly, embryonic stem cells (ES cells) or induced pluripotent stem cells (iPS cells) are appropriate as the pluripotent stem cells.

Embryonic stem cells (ES cells) used in an embodiment of the present disclosure are mammal-derived ES cells. For example, it is possible to use ES cells derived from a rodent such as a mouse or a primate such as a human. Particularly preferably, mouse- or human-derived ES cells are used. ES cells are cells of stem cell lines generated from internal cell mass belonging to a part of an embryo in the blastocyst stage that is the initial stage of animal development. ES cells can grow almost infinitely while maintaining pluripotent differentiation capacity to theoretically differentiate into all tissues. As ES cells, for example, it is possible to use cells in which a reporter gene is introduced in the vicinity of the Pdxl gene in order to facilitate the confirmation of the degree of differentiation. For example, the 129/Sv-derived ES cell line having cells in which the LacZ gene is incorporated at the Pdxl locus or the ES cell line SK7 having the GFP reporter transgene under Pdx1 promoter regulation can be used. Alternatively, it is also possible to use the ES cell line PH3 having the mRFP1 reporter transgene under Hnf3βendoderm-specific enhancer fragment regulation and the GFP reporter transgene under Pdxl promoter regulation. It is also possible to use the ES cell lines SEES1, SEES2, SEES3, SEES4, SEES5, SEES6, and SEES7, which were generated in the Department of Cell Engineering, Department of Reproductive Biology of the National Center for Child Health and Development (NCCHD) disclosed in Akutsu H, et al. Regen Ther. 2015; 1:18-29, and cell lines obtained by introducing additional genes into these ES cell lines.

Induced pluripotent stem cells (iPS cells) used in an embodiment of the present disclosure are pluripotent cells obtained by reprogramming somatic cells. Production of induced pluripotent stem cells have been achieved by several groups such as the group led by Professor Shinya Yamanaka at Kyoto University, the group of Rudolf Jaenisch et al. at the Massachusetts Institute of Technology, the group of James Thomson et al. at the University of Wisconsin-Madison, and the group of Konrad Hochedlinger et al. at Harvard University. For example, International publication no. WO2007/069666 discloses nuclear reprogramming factors for somatic cells containing gene products of the Oct family gene, the Klf family gene, and the Myc family gene and nuclear reprogramming factors for somatic cells containing gene products of the Oct family gene, the Klf family gene, the Sox family gene, and the Myc family gene, and further discloses a method for producing induced pluripotent stem cells by nuclear reprogramming of somatic cells, comprising a step of bringing nuclear reprogramming factors into contact with somatic cells.

Types of somatic cells used for preparing iPS cells are not particularly limited, and arbitrary somatic cells can be used. In other words, somatic cells according to an embodiment of the present disclosure include all cells constituting the living body other than germ cells, and may be differentiated somatic cells or undifferentiated stem cells. The origin of somatic cells may be, but is not particularly limited to, any of mammals, birds, fish, reptiles, and amphibians. It is preferably a mammal (for example, a rodent such as a mouse or a primate such as a human) and particularly preferably a mouse or a human. In addition, in a case in which human somatic cells are used, somatic cells of either a fetus, newborn, or adult may be used. Specific examples of somatic cells include fibroblasts (e.g., skin fibroblasts), epithelial cells (e.g., gastric epithelial cells, hepatic epithelial cells, and alveolar epithelial cells), endothelial cells (e.g., blood vessel cells and lymph vessel cells), nerve cells (e.g., neurons and glial cells), pancreatic cells, blood cells, bone marrow cells, muscle cells (e.g., skeletal muscle cells, smooth muscle cells, and cardiomyocytes), hepatic parenchymal cells, nonhepatic parenchymal cells, adipocytes, osteoblasts, cells constituting periodontal tissue (e.g., periodontal ligament cells, cementoblasts, gingival fibroblasts, osteoblasts), and cells constituting the kidneys, eyes, or ears.

The term “iPS cells” refers to stem cells having long-term self-renewal ability under predetermined culture conditions (for example, under conditions for culturing ES cells) and multilineage potential capable of differentiating into ectoderm, mesoderm, and endoderm under predetermined differentiation induction conditions. In addition, iPS cells according to an embodiment of the present disclosure may be stem cells having ability to form a teratoma when transplanted into a test animal such as a mouse.

In order to produce iPS cells from somatic cells, first, at least one type of reprogramming gene is introduced into somatic cells. The term “reprogramming gene” refers to a gene encoding a reprogramming factor functioning to reprogramming somatic cells to iPS cells. Specific examples of a combination of reprogramming genes include, but are not limited to, the following combinations.
(i) Oct gene, Klf gene, Sox gene, Myc gene
(ii) Oct gene, Sox gene, NANOG gene, LIN28 gene
(iii) Oct gene, Klf gene, Sox gene, Myc gene, hTERT gene, SV40 largeT gene
(iv) Oct gene, Klf gene, Sox gene

### <2. Non-Cell-Adhesive Part and Cell-Adhesive Part of Cell Culture Substrate>

A cell culture substrate used in an embodiment of the present disclosure has a surface comprising a cell culture part.

The cell culture part comprises a non-cell-adhesive part, and a cell-adhesive part extending continuously or intermittently along the periphery of the non-cell-adhesive part and surrounding the non-cell-adhesive part.

One or more such cell culture parts are contained on the surface of the cell culture substrate. When two or more cell culture parts are contained, each individual of the cell culture parts may have the features described above.

The non-cell-adhesive part in the cell culture part may also be referred to as a “second non-cell-adhesive part” or a “central part” in order to distinguish it from a non-cell-adhesive part (first non-cell-adhesive part mentioned later) present in a portion other than the cell culture part.

Specifically, the cell culture substrate used in an embodiment of the present disclosure has the non-cell-adhesive part and the cell-adhesive part formed on the surface thereof so as to assume predetermined shapes.

Hereinafter, a portion other than the cell culture part in the cell culture substrate may also be referred to as a “support substrate” in order to describe features, other than the cell culture part comprising the non-cell-adhesive part and the cell-adhesive part, of the cell culture substrate used in an embodiment of the present disclosure. In other words, the cell culture substrate used in one or more embodiments of the present disclosure comprises a support substrate having a surface comprising the one or more cell culture parts.

Accordingly, embodiments of the support substrate and features other than the shapes of the non-cell-adhesive part and the cell-adhesive part will first be described below.

A support substrate used for a cell culture substrate is not particularly limited as long as the support substrate is formed with a material that allows formation of a non-cell-adhesive part and a cell-adhesive part on the surface thereof. Specific examples of a substrate include: support substrates containing inorganic materials such as glass, a metal, ceramic, and silicon; and organic materials represented by elastomers and plastics (e.g., polystyrene resin, polyester resin, polyethylene resin, polypropylene resin, ABS resin, nylon, acrylic resin, fluororesin, polycarbonate resin, polyurethane resin, methylpentene resin, phenol resin, melamine resin, epoxy resin, and vinyl chloride resin). Particularly, a glass substrate is preferably used as a support substrate. The shape of a support substrate is also not limited, and examples thereof include a flat shape such as a flat substrate, a flat membrane, a film, or a porous membrane, and a three-dimensional shape such as a cylinder, a stamp, a multi-well substrate, or a micro flow path.

According to an embodiment of the present disclosure, the term “cell adhesiveness” means the strength of adhering cells, that is to say, the ease of cell adhesion. The term “cell-adhesive part” refers to a region on the surface with favorable cell adhesiveness, and the term “non-cell-adhesive part” refers to a region on the surface with poor cell adhesiveness. Accordingly, when cells are seeded on the surface on which the cell-adhesive part and the non-cell-adhesive part are arranged with a predetermined pattern, cells adhere to the cell-adhesive parts but not to the non-cell-adhesive part, allowing the cells to be arranged in such pattern on the surface of the cell culture substrate.

The term “cell-adhesive part” is defined as a portion to which cells (preferably stem cells) to be actually cultured adhere when seeded on a cell culture substrate. The term “non-cell-adhesive part” is defined as a portion to which cells (preferably stem cells) to be actually cultured do not adhere when seeded. When cells are seeded on a cell culture substrate, the cell culture substrate may have a surface with enhanced cell adhesiveness by coating with a protein or the like. Specific examples of “stem cells” are as described herein. The non-cell-adhesive part may be covered with cells that have adhered to the cell-adhesive part and grown.

As an indicator for judging whether to be a cell-adhesive part or to be a non-cell-adhesive part, the cell adhesion/spreading rate when actually culturing cells can be used. The surface of a cell-adhesive part having cell adhesiveness is preferably a surface having a cell adhesion/spreading rate of 60% or more and more preferably a surface having a cell adhesion/spreading rate of 80% or more. When the cell adhesion/spreading rate is high, cells can be cultured efficiently. The cell adhesion/spreading rate according to an embodiment of the present disclosure can be defined as a proportion of adhering/spread cells at a time point when cells to be cultured at a seeding density in a range of 4000 cells/cm² or more to less than 30000 cells/cm² have been seeded on a surface of a measurement subject, stored in an incubator at 37° C. and a CO₂ concentration of 5%, and cultured for 14.5 hours ({(the number of adhering cells)/(the number of seeded cells)}×100(%)).

In the above measurement, cell seeding is conducted in a manner such that cells suspended in a 10% FBS-containing DMEM medium are seeded on a surface to be measured, and then, the surface to be measured on which cells have been seeded is slowly shaken to allow the cells to be distributed as uniformly as possible. Further, the cell adhesion/spreading rate is measured after changing the medium immediately before the measurement to remove non-adhering cells. Upon measurement of the cell adhesion/spreading rate, areas other than areas where the cell presence density is likely to be specific (for example, the center of a predetermined area where the presence density tends to be high and the periphery of a predetermined area where the presence density tends to be low) are determined to be measurement areas.

The cell-adhesive part may be a region in which a cell-adhesive layer is formed on the surface of the support substrate. When the surface of the support substrate has cell adhesiveness (e.g., the surface of a glass substrate), the cell-adhesive part may be a region on which the surface of the support substrate is exposed. A region on which the surface having cell adhesiveness of the support substrate is exposed is preferred. The non-cell-adhesive part can be a region in which a non-cell-adhesive layer is formed on the surface of the support substrate. The cell-adhesive part and the non-cell-adhesive part can be formed using various materials and methods. Preferably, the non-cell-adhesive part is a portion in which the surface of the support substrate is coated with a non-cell-adhesive layer such as a layer containing a hydrophilic organic compound such as a hydrophilic polymer. The average thickness of the non-cell-adhesive layer constituting the non-cell-adhesive part is, as described in Patent Literature 4, preferably 0.8 nm to 500 μm, more preferably 0.8 nm to 100 μm, further preferably 1 nm to 10 μm and most preferably 1.5 nm to 1 μm. In a case in which the average thickness is 0.8 nm or more, protein adsorption and cell adhesion are unlikely to be affected by areas other than areas covered with the non-cell-adhesive layer on the support substrate, which is preferable. In addition, in a case in which the average thickness is 500 μm or less, coating is relatively easy. Further, as described in Patent Literature 5, in the case of forming a non-cell-adhesive layer with a layer of polyethylene glycol, one example of its film thickness can include 5 nm to 10 nm. Specific examples of the hydrophilic organic compound are as mentioned later.

A method described in Patent Literature 4 and Non Patent Literature 10 can be used as a method for producing a cell culture substrate containing polyethylene glycol (PEG) as a hydrophilic polymer in a non-cell-adhesive layer.

The following two embodiments are particularly preferable for a method for forming the cell-adhesive part and the non-cell-adhesive part.

In the first embodiment, a non-cell-adhesive layer is formed on the surface of a support substrate. Then, a part of the non-cell-adhesive layer is subjected to a predetermined treatment so that cell adhesiveness is exerted to prepare a cell-adhesive part. Specifically, an exemplary method involves forming a non-cell-adhesive hydrophilic film containing a hydrophilic organic compound such as a hydrophilic polymer, as a non-cell-adhesive layer on the surface of a support substrate, and then selectively subjecting a part of the hydrophilic film serving as a non-cell-adhesive layer to oxidation treatment and/or decomposition treatment to modify the part into a cell-adhesive part having cell adhesiveness. In this embodiment, the cell-adhesive part is prepared by forming a non-cell-adhesive hydrophilic film, and then, applying oxidation treatment and/or decomposition treatment to a site where cell adhesion is desired so as to convert the site to a site having cell adhesiveness. In the cell culture substrate formed according to the first embodiment, the non-cell-adhesive part is a portion in which the surface of the support substrate is coated with a layer containing a hydrophilic organic compound such as a hydrophilic polymer, and the cell-adhesive part is a portion on which the surface of the support substrate is exposed by removing the layer containing a hydrophilic organic compound such as a hydrophilic polymer through oxidation treatment and/or decomposition treatment, or a portion coated with a layer modified so as to have cell adhesiveness (=cell-adhesive layer) from the layer containing a hydrophilic organic compound such as a hydrophilic polymer in response to oxidation treatment and/or decomposition treatment.

In the second embodiment, the cell-adhesive part and the non-cell-adhesive part depend on a high or low density of an organic compound on the surface of a support substrate. In the cell culture substrate formed according to the second embodiment, the cell-adhesive part is a surface having a low density of a hydrophilic organic compound such as a hydrophilic polymer (which also includes the absence of the hydrophilic organic compound), and the non-cell-adhesive part is a surface having a high density of a hydrophilic organic compound such as a hydrophilic polymer. The second embodiment exploits the fact that the surface of the support substrate containing a hydrophilic organic compound such as a hydrophilic polymer at a high density has non-cell-adhesiveness, whereas the surface of the support substrate having a low density of the compound has cell adhesiveness. A first region likely to be bonded to the compound and a second region unlikely to be bonded to the compound are established on a support substrate surface, and a film of the compound is formed on the substrate surface. As a result, the first region becomes a non-cell-adhesive part, and the second region becomes a cell-adhesive region. Alternatively, a part of the support substrate surface is selectively masked with a photoresist, and a film of the hydrophilic organic compound is formed on an unmasked region to form a non-cell-adhesive part. Then, the masking is removed so that the surface of the support substrate can be exposed to form a cell-adhesive part.

Instead of these embodiments, a support substrate having a non-cell-adhesive surface (which may be a surface of a non-cell-adhesive layer) is prepared, and a part of the surface may be coated by patterning with a cell-adhesive protein such as collagen or fibronectin to form a cell-adhesive pattern. Alternatively, a support substrate having a cell-adhesive surface (which may be a surface of a cell-adhesive layer) is prepared, and a part of the surface may be coated with a non-cell-adhesive resin such as silicone rubber (e.g., KEIJU(R) manufactured by Mitsubishi Chemical Corp.) to prepare a cell adhesive pattern as a remnant. Alternatively, a support substrate provided with a conductive layer having a predetermined pattern on the surface is prepared, and the surface of the support substrate is covered with a non-cell-adhesive layer. The coating with the non-cell-adhesive layer on the conductive layer is detached by voltage application to the conductive layer, and the conductive layer thus exposed may be used as a cell-adhesive part (for the details, see JP Patent Publication (Kokai) No. 2012-120443 A (2012) and JP Patent Publication (Kokai) No. 2013-179910 A (2013)).

Hereinafter, the first and second embodiments will be described in order in which a cell-adhesive part and a non-cell-adhesive part are formed on a support substrate surface to produce a cell culture substrate having a surface comprising the cell-adhesive part and the non-cell-adhesive part.

First, the first embodiment will be described.

In the first embodiment, a hydrophilic film containing a hydrophilic organic compound, preferably a hydrophilic polymer, is first established as a non-cell-adhesive layer on a support substrate surface, The hydrophilic film is a thin film having water solubility or water swellability, and is not particularly limited as long as it has non-cell-adhesiveness before being oxidated and/or decomposed and an exposed surface of the support substrate after oxidation and/or decomposition or the surface of the thin film modified in response to oxidation treatment and/or decomposition treatment exhibits cell adhesiveness.

When the non-cell-adhesive layer is a hydrophilic film formed from a hydrophilic organic compound, it is preferable to establish a bonding layer, if necessary, between the surface of the support substrate and the hydrophilic film. The bonding layer is preferably a layer containing a material having a functional group capable of being bonded (bonding functional group) to a functional group of the organic compound in the hydrophilic film. Examples of a combination of the bonding functional group of the material of the bonding layer and the functional group of the hydrophilic organic compound include an epoxy group and a hydroxy group, phthalic anhydride and a hydroxy group, a carboxyl group and N-hydroxysuccinimide, a carboxyl group and carbodiimide, and an amino group and glutaraldehyde. Either of the functional groups in each combination may be present in the bonding layer. In these methods, a bonding layer is formed with a material having a predetermined functional group on a support substrate before coating with a hydrophilic organic compound. In the non-cell-adhesive layer, in one example of a silane coupling agent having a terminal epoxy group for use as a material having a bonding functional group, the water contact angle on the surface of the bonding layer before formation of a thin film of a hydrophilic organic compound is typically 45° or more, desirably 47° or more. Such a bonding layer is obtained by forming a coating of a material having a bonding functional group on the surface of a support substrate.

Examples of the hydrophilic organic compound include hydrophilic polymers (including hydrophilic oligomers), water-soluble organic compounds, surface active materials, and amphipathic materials. A hydrophilic polymer is particularly preferable.

Specific examples of the hydrophilic polymer can include polyalkylene glycol, zwitterionic polymers having a phospholipid polar group, polyacrylamide, polyacrylic acid, polymethacrylic acid, polyvinyl alcohol, and polysaccharides. Specific examples of these hydrophilic polymers also include their derivative forms. Examples of the molecular shape of the hydrophilic polymer can include linear or branched polymers and dendrimers.

Specifically, the polyalkylene glycol is preferably polyethylene glycol, polypropylene glycol, or a copolymer of polyethylene glycol and polypropylene glycol, for example, Pluronic F108 or Pluronic F127.

Specifically, the zwitterionic polymer having a phospholipid polar group is preferably poly(methacryloyloxyethyl phosphorylcholine) (=MPC polymer), a copolymer of methacryloyloxyethyl phosphorylcholine and an acrylic monomer, or the like.

Specific examples of the polyacrylamide can include poly(N-isopropylacrylamide).

Specific examples of the polymethacrylic acid can include poly(-hydroxyethyl methacrylate).

Specific examples of the polysaccharide can include dextran and heparin.

It is desirable that the surface of the support substrate having a non-cell-adhesive layer should have high non-cell-adhesiveness in a state coated with the non-cell-adhesive layer and an exposed surface of the support substrate after oxidation treatment and/or decomposition treatment of the non-cell-adhesive layer, or the surface of a layer formed by modifying the non-cell-adhesive layer through oxidation treatment and/or decomposition treatment should exhibit cell adhesiveness.

The hydrophilic polymer is particularly preferably polyethylene glycol (PEG). PEG includes at least an ethylene glycol chain (EG chain) comprising at least one ethylene glycol unit ((CH₂)₂—O), which may be either linear or branched chain. The ethylene glycol chain has a structure expressed by, for example, the following formula:

—((CH₂)₂—O)m-

(m is an integer indicating the degree of polymerization).
m is preferably an integer of 1 to 13 and more preferably an integer of 1 to 10.

PEG also includes ethylene glycol oligomer. In addition, PEG may be functional-group-containing PEG. Examples of a functional group include an epoxy group, a carboxyl group, an N-hydroxysuccinimide group, a carbodiimide group, an amino group, a glutaraldehyde group, and a (meth)acryloyl group. A functional group is optionally introduced via a linker, preferably at the end of the chain. Examples of functional-group-containing PEG include PEG(meth)acrylate and PEG di(meth)acrylate.

The cell-adhesive part can be formed by applying oxidation treatment and/or decomposition treatment to a non-cell-adhesive layer containing a hydrophilic organic compound formed on the surface of a support substrate so that the surface of the support substrate having cell adhesiveness is exposed or so that the non-cell-adhesive layer is modified and converted into a cell-adhesive layer.

According to an embodiment of the present disclosure, the word “oxidation” has a narrow meaning and refers to a reaction in which an organic compound reacts with oxygen and the content of oxygen becomes larger than that before the reaction.

According to an embodiment of the present disclosure, the word “decomposition” refers to a change in which a bond of an organic compound is cleaved to generate two or more organic compounds from the organic compound. Typically, “decomposition treatment” is caused by, but is not limited to, oxidation treatment, ultraviolet irradiation, or other treatments. In a case in which “decomposition treatment” is accompanied by oxidation (i.e., oxidation decomposition), “decomposition treatment” and “oxidation treatment” refer to the same treatment. The removal of a non-cell-adhesive layer by decomposition is also included in “decomposition treatment”.

Decomposition by ultraviolet irradiation means that an organic compound absorbs ultraviolet rays and decomposes via an excited state. When a system in which an organic compound is present together with molecular species containing oxygen (e.g., oxygen or water) with ultraviolet rays, ultraviolet rays are absorbed by the organic compound and decomposition occurs, and in some cases, the molecular species is activated to react with the organic compound. The latter reaction can be classified as “oxidation.” A reaction in which an organic compound is decomposed via oxidation caused by the activated molecular species can be classified as “decomposition by oxidation” rather than “decomposition by ultraviolet irradiation.”

As described above, “oxidation treatment” and “decomposition treatment” may be duplicated as operations, and therefore, they cannot be distinguished clearly. In view of this, the term “oxidation treatment and/or decomposition treatment ” is used herein.

Next, the second embodiment will be described. In the cell culture substrate formed according to the second embodiment, the cell-adhesive part in the surface of the support substrate is a surface having a low density of a hydrophilic organic compound such as a hydrophilic polymer (which also includes the absence of the hydrophilic organic compound), and the non-cell-adhesive part is a surface having a high density of a hydrophilic organic compound. In other words, the cell-adhesive part and the non-cell-adhesive part differ in the density of a hydrophilic organic compound. A higher density thereof tends to render cells more unlikely to adhere. In the cell-adhesive part, the density of the hydrophilic organic compound is low to an extent that cells can adhere thereto. Preferred examples of the hydrophilic organic compound and the hydrophilic polymer are as already mentioned about the first embodiment.

In the second embodiment, in the case of forming a cell-adhesive part and a non-cell-adhesive part with a hydrophilic film having a controlled density, it is preferable to form a bonding layer, if necessary, on a support substrate in order to enhance adhesion to the support substrate, and then form a hydrophilic film made of a hydrophilic organic compound. The bonding layer is preferably a layer containing a material containing a functional group capable of being bonded (bonding functional group) to a functional group of the hydrophilic organic compound. Examples of a combination of the functional group of the material of the bonding layer and the functional group of the hydrophilic organic compound include an epoxy group and a hydroxy group, phthalic anhydride and a hydroxy group, a carboxyl group and N-hydroxysuccinimide, a carboxyl group and carbodiimide, and an amino group and glutaraldehyde. Either of the functional groups in each combination may be present in the bonding layer. In these methods, a bonding layer is formed with a material having a predetermined functional group on a support substrate before coating with a hydrophilic material. The density of the material in the bonding layer is an important factor for defining a bonding force. The density can be readily evaluated based on the water contact angle as an index on the surface of the bonding layer. The water contact angle is a value measured 30 seconds after dropwise addition of pure water from a microsyringe using CA-Z manufactured by Kyowa Interface Science Co., Ltd.

In the bonding layer of the cell-adhesive part, the material having a bonding functional group has a low density. In the cell-adhesive part, in an example of a silane coupling agent having a terminal epoxy group for use as a material having a bonding functional group constituting the bonding layer, the water contact angle on the surface of the bonding layer before formation of a thin film of a hydrophilic organic compound is typically 10° to 43°, desirably 15° to 40°. Examples of a method for forming such a bonding layer include a method of forming a coating of a material having a bonding functional group (bonding layer) on the surface of a support substrate, followed by oxidation treatment and/or decomposition treatment of the surface of the bonding layer. Examples of an oxidation treatment and/or decomposition treatment method for a bonding layer surface include a method for irradiating a bonding layer surface with ultraviolet rays, a method for treating a bonding layer surface with a photocatalyst, and a method for treating a bonding layer surface with an oxidizing agent. The whole bonding layer surface may be subjected to oxidation treatment and/or decomposition treatment, or the bonding layer surface may be partially treated. The partial treatment can be performed by using a mask such as a photomask or a stencil mask or using a stamp. In addition, oxidation treatment and/or decomposition treatment may be carried out by a direct drawing method such as a method using laser such as ultraviolet laser. As for conditions, etc., the same conditions as in a method for forming a cell-adhesive part by the oxidation treatment and/or decomposition treatment of a hydrophilic film can be applied. The cell-adhesive part can be formed by forming a thin film of a hydrophilic organic compound on the bonding layer thus formed.

In the bonding layer of the non-cell-adhesive part, the material having a bonding functional group has a high density. In the non-cell-adhesive part, in an example of a silane coupling agent having a terminal epoxy group for use as a material having a bonding functional group, the water contact angle on the surface of the bonding layer before formation of a thin film of a hydrophilic organic compound is typically 45° or more, desirably 47° or more. Such a bonding layer is obtained by forming a coating of a material having a bonding functional group on the surface of a support substrate. In the case of partially subjecting a bonding layer surface to oxidation treatment and/or decomposition treatment, a residual portion that has not undergone the treatment becomes a bonding layer having the water contact angle. The non-cell-adhesive layer can be formed by forming a thin film of a hydrophilic organic compound on the bonding layer thus formed.

In the second embodiment, a part of the support substrate surface is selectively masked with a photosensitive photoresist, and a film of the hydrophilic organic compound is formed on an unmasked region to form a non-cell-adhesive part. Then, the masking is removed so that the surface of the support substrate can be exposed to form a cell-adhesive part.

Subsequently, features of a cell-adhesive part and a non-cell-adhesive part formed according to the first or second embodiment described above or other methods will be further described.

The amount of carbon in a cell-adhesive part (including a bonding layer when there is a bonding layer) is preferably lower than the amount of carbon in a non-cell-adhesive part (including a bonding layer when there is a bonding layer). Specifically, the amount of carbon in a cell-adhesive part is preferably 20% to 99% of the amount of carbon in a non-cell-adhesive part. This range is appropriate, particularly, when the thickness of a hydrophilic organic compound layer (the total thickness of a bonding layer and the hydrophilic film when there is a bonding layer) contained in a cell-adhesive part or a non-cell-adhesive part is 10 μm or less. “Amount of carbon (atomic concentration, %)” is as defined below.

In addition, the proportion of carbon bonded to oxygen (%) with respect to carbon present in a cell-adhesive part (including a bonding layer when there is a bonding layer) is preferably smaller than the proportion of carbon bonded to oxygen (%) with respect to carbon present in a non-cell-adhesive part (including a bonding layer when there is a bonding layer). Specifically, the proportion of carbon bonded to oxygen (%) with respect to carbon present in a cell-adhesive part is preferably 35% to 99% of the proportion of carbon bonded to oxygen (%) with respect to carbon present in a non-cell-adhesive part. This range is appropriate, particularly, when the thickness of a hydrophilic film (the total thickness of a bonding layer and the hydrophilic film when there is a bonding layer) is 10 μm or less. “Proportion of carbon bonded to oxygen (atomic concentration, %)” is as defined below.

Contact angle measurement, ellipsometry, atomic force microscopy, electron microscopy, Auger electron spectroscopy, X-ray photoelectron spectroscopy, various mass spectrometry methods, or the like can be used as an approach of evaluating a hydrophilic organic compound layer (including a bonding layer when there is a bonding layer) contained in a cell-adhesive part or a non-cell-adhesive part. Among these approaches, X-ray photoelectron spectroscopy (XPS/ESCA) has the best quantitativeness. A relative quantitative value is determined by this measurement method and is generally calculated in atomic concentration (%). Hereinafter, an X-ray photoelectron spectroscopy method according to an embodiment of the present disclosure will be described in detail.

“The amount of carbon” in a cell-adhesive part and a non-cell-adhesive part is defined as “the amount of carbon calculated from the analysis value of the C1s peak obtained by using an X-ray photoelectron spectrometer”. According to an embodiment of the present disclosure, “the proportion of carbon bonded to oxygen” in a cell-adhesive part and a non-cell-adhesive part is defined as “the proportion of carbon bonded to oxygen calculated from the analysis value of the C1s peak obtained by using an X-ray photoelectron spectrometer.” Specific measurement can be carried out as described in JP Patent Publication (Kokai) No. 2007-312736 A (2007).

### <3. Feature of Shape of Cell Culture Part in Cell Culture Substrate>

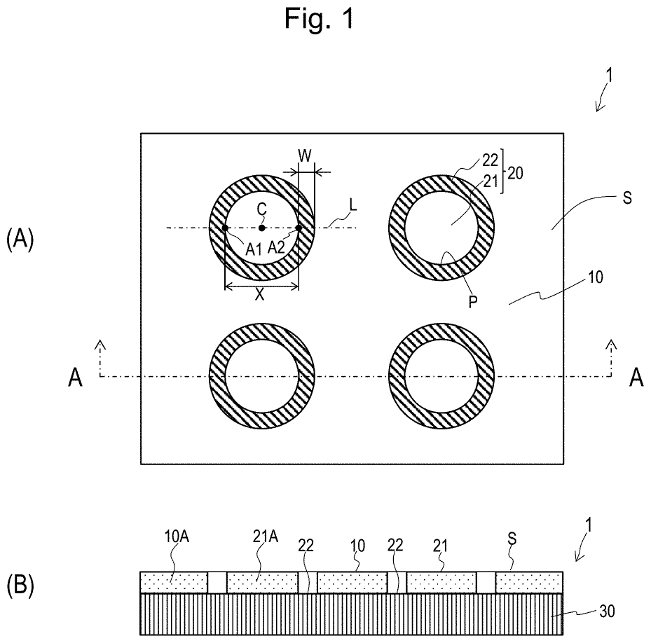

Features of a cell culture substrate used in an embodiment of the present disclosure will be described, mainly, with reference to FIG. 1.

Cell culture substrate 1 used in an embodiment of the present disclosure has surface S comprising cell culture part 20.

The cell culture part 20 has non-cell-adhesive part (central part) 21, and cell-adhesive part 22 extending continuously or intermittently (FIG. 1 shows an example of extending continuously) along periphery P of the non-cell-adhesive part 21 and surrounding the non-cell-adhesive part 21. This embodiment in an example in which one or more cell culture parts 20 are contained on surface S of cell culture substrate 1 and each of the one or more cell culture parts 20 has the features described above.

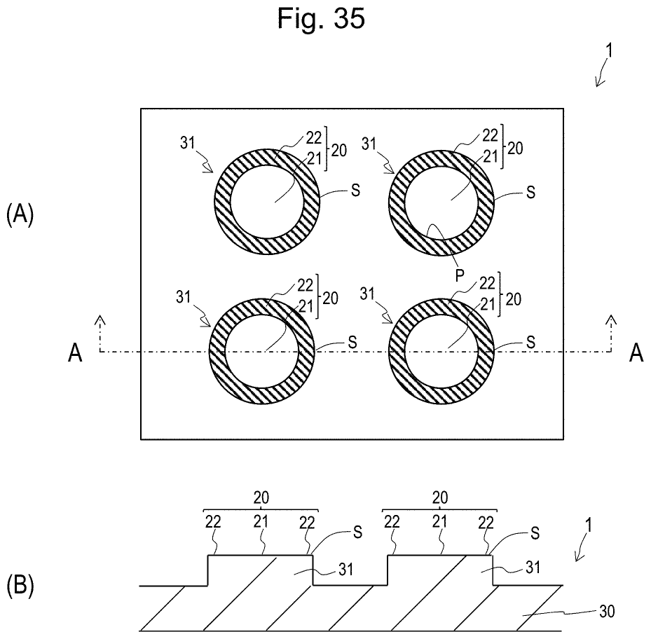

In the example shown in FIG. 1, one or more cell culture parts 20 are scattered in the form of islands in non-cell-adhesive part 10. In this example, the non-cell-adhesive part 10 may also be referred to as “first non-cell-adhesive part”, and the non-cell-adhesive part 21 in the cell-adhesive part 20 may also be referred to as “second non-cell-adhesive part”. In the description below, the “non-cell-adhesive part 21” may also be referred to as “central part 21” or “central part 21 serving as a non-cell-adhesive part”. The first non-cell-adhesive part 10 is not an essential constituent. An example of a cell culture substrate having no first non-cell-adhesive part 10 will be separately described with reference to FIGS. 35 to 37.

In the cell culture substrate 1, a portion in which first non-cell-adhesive part 10 and cell-adhesive part 20 are arranged on the surface is referred to as “support substrate 30”.

In the example shown in FIG. 1, the first non-cell-adhesive part 10 and the central part 21 serving as a second non-cell-adhesive part are the surfaces of first non-cell-adhesive layer 10A and second non-cell-adhesive layer 21A, respectively, disposed on the surface of the support substrate 30.

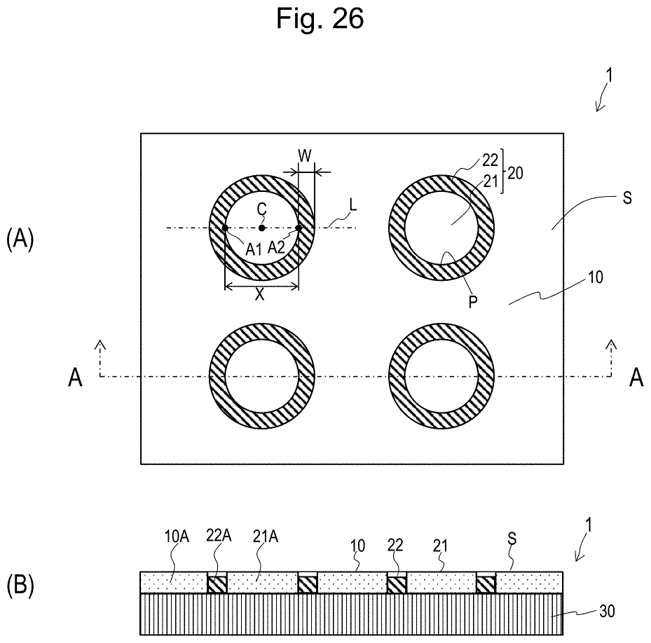

In the example shown in FIG. 1, the cell-adhesive part 22 is an exposed surface of the support substrate 30. As in an example shown in FIG. 26, the cell-adhesive part 22 may be the surface of cell-adhesive layer 22A disposed on the surface of the support substrate 30.

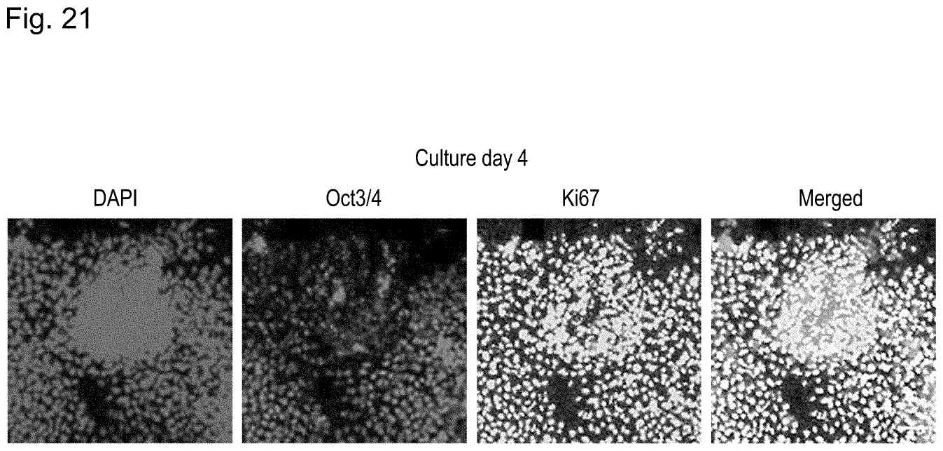

In FIG. 1(B) and FIG. 21(B), the thicknesses of the non-cell-adhesive layer 10A, the non-cell-adhesive layer 21A and the cell-adhesive layer 22A as well as the level difference between the cell-adhesive part 22 and the non-cell-adhesive layer 10A or the non-cell-adhesive layer 21A is emphatically shown for the sake of convenience. However, the thickness and the level difference are sufficiently small with respect to the sizes of cells to be cultured and the cell construct. Therefore, surface S comprising one or more cell culture parts 20 can support cells as a substantially flat surface.

FIG. 1(B) shows an example in which the support substrate 30 is in direct contact with the first non-cell-adhesive layer 10A and the second non-cell-adhesive layer 21A. As already mentioned, a bonding layer may intervene therebetween. Likewise, FIG. 26(B) shows an example in which the support substrate 30 is in direct contact with the first non-cell-adhesive layer 10A, the second non-cell-adhesive layer 21A, and the cell-adhesive layer 22A. As already mentioned, a bonding layer may intervene therebetween.

Specific examples and production methods of the support substrate 30, the first non-cell-adhesive part 10, the first non-cell-adhesive layer 10A, the second cell-adhesive part 21, the second non-cell-adhesive layer 21A, the cell-adhesive part 22, and the cell-adhesive layer 22A are as already mentioned.

The present inventors have found that, surprisingly, when stem cells are cultured on cell culture substrate 1 having such a structure, stem cells attach and grow on cell-adhesive part 22 surrounding non-cell-adhesive part (central part) 21 to form an accumulating portion having dense accumulations of cells and a sac-shaped cell construct (tissue) that can differentiate into intestinal epithelial cells expressing a trophectodermal cell marker is easily formed in the formed accumulating portion of cells. The obtained cell construct comprises small intestinal epithelial cells and has functions as a gut organoid. The present inventors have found that the differentiation induction of stem cells using a cell culture substrate having the structure allows a cell construct comprising small intestinal epithelial cells to be released from the cell culture substrate in a relatively short time and collected and furthermore, collection efficiency is exceedingly high.

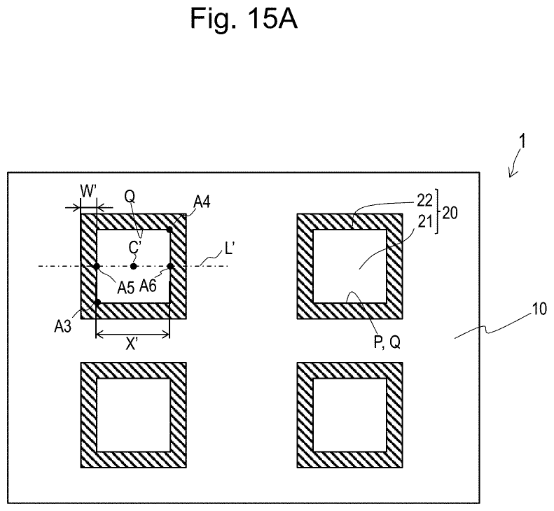

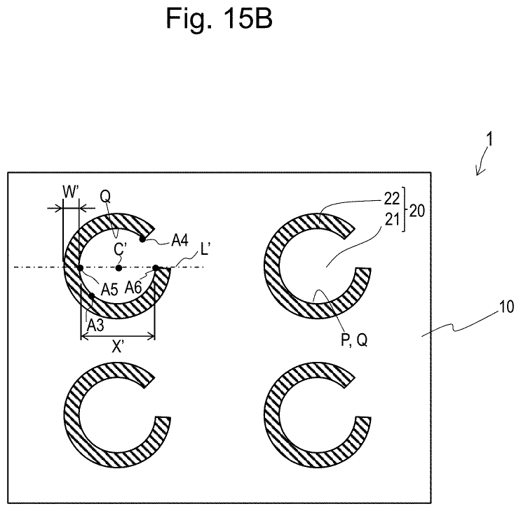

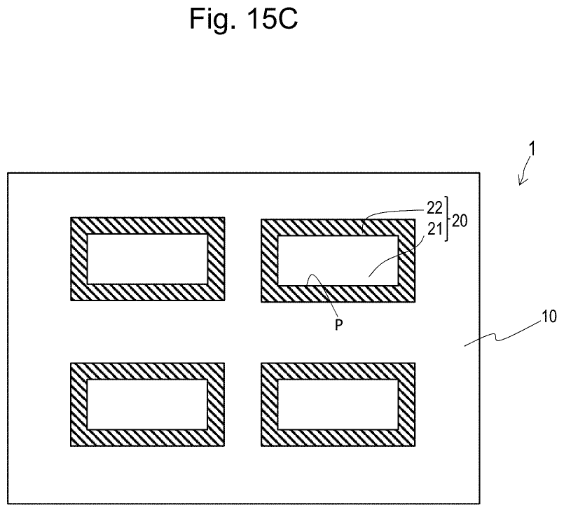

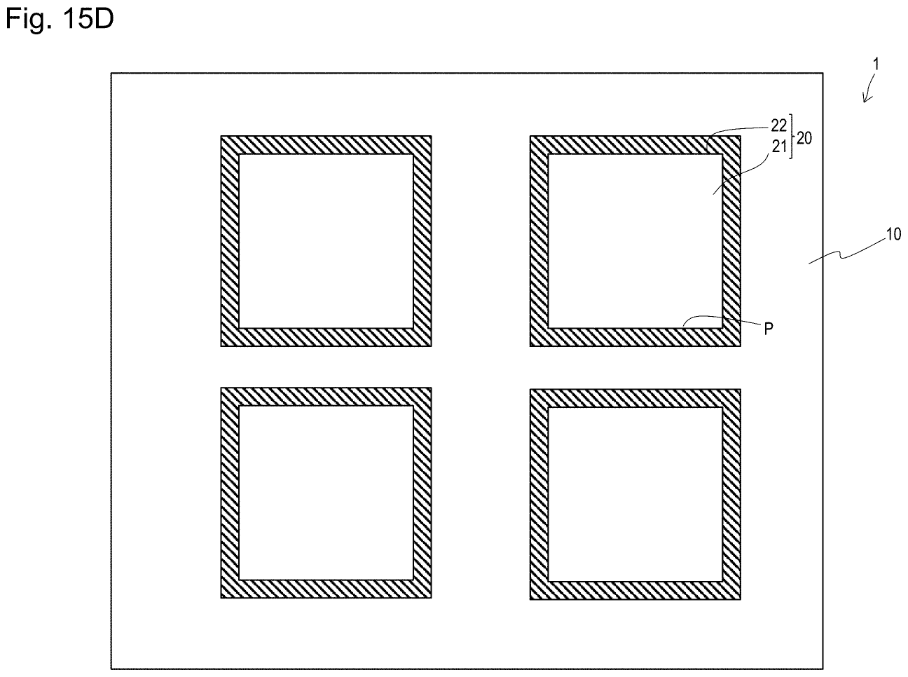

The shape or size of the cell culture part 20 is not particularly limited. In a preferable embodiment, distance X between two points of intersection, A1 and A2, of periphery P of the central part 21 with straight line L passing through barycenter C of the central part 21 is larger than 80 μm and 880 μm or smaller, more preferably 180 μm or larger and 880 μm or smaller, and particularly preferably 180 μm or larger and 600 μm or smaller. If the distance X is too small, a specific sac-shaped structure is unlikely to be obtained on the outer circumference of the cell construct because the central part 21 is immediately covered with trophectodermal cells during growing culture. On the other hand, if the distance X is too large, the production efficiency of the cell construct is reduced because the time required for cells to grow and completely cover the central part 21 is long. When the distance X falls within the above range, a cell construct having a sac-shaped structure can be cultured at high yields in a relatively short time. The distance X refers to the diameter of a circle when the shape of the central part 21 is a circle as shown in FIG. 1 and FIG. 15B. When the circle is a true circle, the distance X is constant regardless of how the straight line L is drawn. When the central part 21 is rectangular as shown in FIG. 15A, 15C or 15D, the distance X is largest with the straight line L drawn in the diagonal direction and is smallest with the straight line L drawn in the shorter direction. According to an embodiment of the present disclosure, preferably, the distance X falls within the above range over the whole circumference (i.e., for any straight lines L drawn).

In another preferable embodiment of the cell culture part 20, width W of the cell-adhesive part 22 along the straight line L passing through barycenter C of the central part 21 is larger than 30 μm and 400 μm or smaller, more preferably 40 μm or larger and 400 μm or smaller, and particularly preferably 60 μm or larger and 300 μm or smaller. If the width W is too small, cells are disadvantageously likely to be detached during culture. For the induction of a sac-shaped cell construct, it is desirable that a plurality of cells should adhere in the width direction of the cell-adhesive part 22 to form an accumulating portion. For this purpose, larger width W is preferable. Therefore, as described above, the width W is preferably 40 μm or larger, more preferably 60 μm or larger. On the other hand, if the width W is too large, a cell construct having a uniform structure is unlikely to be obtained because the density of cells adhering to the cell-adhesive part 22 is likely to be biased and makes it difficult to form an accumulating portion of cells uniform in the width direction. When the width W falls within the above range, a cell construct can be cultured at high yields in a relatively short time. The width W refers to the width of the cell-adhesive part 22 in the diameter direction of a circle when the shape of the central part 21 is a circle as shown in FIG. 1 and FIG. 15B. When circle is a true circle, the width W is constant regardless of how the straight line L is drawn. When the central part 21 is rectangular as shown in FIG. 15A, 15C or 15D, the width W is largest with the straight line L drawn in the diagonal direction and is smallest with the straight line L drawn in the shorter direction. According to an embodiment of the present disclosure, preferably, the width W falls within the above range over the whole circumference (i.e., for any straight lines L drawn).

In an alternative preferable embodiment of the cell culture part 20, ratio X/W of distance X to width W is preferably 0.5 or more, more preferably 1.0 or more, and further preferably 1.3 or more, and is preferably 20.0 or less, more preferably 15.0 or less, and further preferably 10.0 or less. The distance X is a distance between two points of intersection, A1 and A2, of periphery P of the central part 21 with straight line L passing through barycenter C of the central part 21. The width W is a width of the cell-adhesive part 22 along the straight line L. When the ratio X/W falls within the above range, a cell construct can be cultured at high yields in a relatively short time. According to an embodiment of the present disclosure, preferably, the ratio X/W falls within the above range over the whole circumference (i.e., for any straight lines L drawn).

The shape and size of the cell-adhesive part 22 can be defined by midpoint C′, distance X′, width W′, and straight line L′ described below, instead of barycenter C, distance X, width W, and straight line L described above. The midpoint C′, the distance X′, the width W′, and the straight line L′ will be described with reference to FIG. 15A and FIG. 15B. A straight line passing through midpoint C′ between two points, A3 and A4, on an inner circumference Q of the cell-adhesive part 22, opposed to and most distant from each other across the central part 21 is defined as straight line L′. A distance between two points of intersection, A5 and A6, of this straight line L′ with the inner circumference Q of the cell-adhesive part 22 is defined as distance X′. A width of the cell-adhesive part 22 along the straight line L′ passing through midpoint C′ is defined as width W′.

The distance X′ is preferably larger than 80 μm and 880 μm or smaller, more preferably 180 μm or larger and 880 μm or smaller, particularly preferably 180 μm or larger and 600 μm or smaller, and particularly preferably 180 μm or larger and 500 μm or smaller. If the distance X′ is too small, a specific sac-shaped structure is unlikely to be obtained on the outer circumference of the cell construct because the central part 21 is immediately covered with cells during growing culture. On the other hand, if the distance X′ is too large, the production efficiency of the cell construct is reduced because the time required for cells to grow and completely cover the central part 21 is long. When the distance X′ falls within the above range, a cell construct having a sac-shaped structure can be cultured at high yields in a relatively short time. The distance X′ refers to the diameter of a circle when the shape of the central part 21 is a circle as shown in FIG. 1 and FIG. 15B. When the circle is a true circle, the distance X′ is constant regardless of how the straight line L′ is drawn. When the central part 21 is rectangular as shown in FIG. 15A, 15C or 15D, the distance X′ is largest with the straight line L′ drawn in the diagonal direction and is smallest with the straight line L′ drawn in the shorter direction. According to an embodiment of the present disclosure, preferably, the distance X′ falls within the above range over the whole circumference (i.e., for any straight lines L′ drawn).

The width W′ is preferably larger than 30 μm and 400 μm or smaller, more preferably 40 μm or larger and 400 μm or smaller, and particularly preferably 60 μm or larger and 300 μm or smaller. If the width W′ is too small, cells are disadvantageously likely to be detached during culture. For the induction of a sac-shaped cell construct, it is desirable that a plurality of cells should adhere in the width direction of the cell-adhesive part 22 to form an accumulating portion. For this purpose, larger width W′ is preferable. Therefore, as described above, the width W′ is preferably 40 μm or larger, more preferably 60 μm or larger. On the other hand, if the width W′ is too large, a cell construct having a uniform structure is unlikely to be obtained because the density of cells adhering to the cell-adhesive part 22 is likely to be biased and makes it difficult to form an accumulating portion of cells uniform in the width direction. When the width W′ falls within the above range, a cell construct can be cultured at high yields in a relatively short time. The width W′ refers to the width of the cell-adhesive part 22 in the diameter direction of a circle when the shape of the central part 21 is a circle as shown in FIG. 1 and FIG. 15B. When the circle is a true circle, the width W′ is constant regardless of how the straight line L′ is drawn. When the central part 21 is rectangular as shown in FIG. 15A, 15C or 15D, the width W′ is largest with the straight line L′ drawn in the diagonal direction and is smallest with the straight line L′ drawn in the shorter direction. According to an embodiment of the present disclosure, preferably, the width W′ falls within the above range over the whole circumference (i.e., for any straight lines L′ drawn).

Ratio X′/W′ is preferably 0.5 or more, more preferably 1.0 or more, and further preferably 1.3 or more, and is preferably 20.0 or less, more preferably 15.0 or less, and further preferably 10.0 or less. When the ratio X′/W′ falls within the above range, a cell construct can be cultured at high yields in a relatively short time. According to an embodiment of the present disclosure, preferably, the ratio X′/W′ falls within the above range over the whole circumference (i.e., for any straight lines L′ drawn).

In FIG. 1 and FIG. 15B, the central part 21 is round, and the cell-adhesive part 22 has an annular shape concentrically surrounding the round central part 21. This high symmetric fashion is particularly preferable for obtaining a uniform cell construct. However, the present invention is not limited by such an example, and an inner outline and an outer outline may have a rectangular shape, as shown in FIG. 15A, FIG. 15C, or FIG. 15D, in which the central part 21 is rectangular (square or rectangle) and the cell-adhesive part 22 is disposed along periphery P of the rectangular central part 21. Although not shown, the central part may be oval, and the cell-adhesive part may have an oval annular shape extending along the central part. In the examples described above, the inner outline and the outer outline of the cell-adhesive part have similar shapes, though not limited thereto. For example, the inner outline of the cell-adhesive part (i.e., the outer outline of the central part) may be polygonal (e.g., rectangular), and the outer outline of the cell-adhesive part may be round or oval; or alternatively, the inner outline of the cell-adhesive part (i.e., the outer outline of the central part) may be round or oval, and the outer outline of the cell-adhesive part may be polygonal (e.g., rectangular). Alternatively, the central part 21 may be semicircular.

In the examples of FIG. 1, FIG. 15A, FIG. 15C, and FIG. 15D, the cell-adhesive part 22 extends continuously along periphery P of the central part 21 serving as a second non-cell-adhesive part and surrounds the central part 21 over the whole circumference. However, the cell-adhesive part may have a shape extending intermittently. Specifically, as shown in the example of FIG. 15B, the cell-adhesive part 22 extends intermittently along periphery P of the central part 21 serving as a second non-cell-adhesive part and surrounds the central part 21. Even in such a structure, cells that have adhered on the cell-adhesive part 22 can form a tissue through growth so as to connect the gap portion of the cell-adhesive part 22. In an embodiment in which the cell-adhesive part 22 extends intermittently along periphery P of the central part 21, the discontinued portion has a length of preferably ½ or less, more preferably ¼ or less, further preferably ⅙ or less, and furthermore preferably ⅛ or less, of the complete periphery P of the central part 21 per location. In the case of including a plurality of discontinued portions, the discontinued portions have a total length of preferably ½ or less, more preferably ¼ or less, further preferably ⅙ or less, and furthermore preferably ⅛ or less, of the complete periphery P of the central part 21.

A cell culture substrate used in an embodiment of the present disclosure has a structure where the cell-adhesive part extends so as to surround the non-cell-adhesive central part, whereby cells that attach and grow on this cell-adhesive part become dense and are more likely to differentiate into cells having the properties of trophectodermal cells while grown cells are likely to accumulate. As a result, a sac-shaped cell construct in which cells having the properties of trophectodermal cells are distributed on the outer circumference can be efficiently obtained.

By contrast, in an approach described in Patent Literature 2 and Non Patent Literature 3, a cell-adhesive part has a round shape which causes cells having the properties of trophectodermal cells to spread into the inside through growth. Thus, a sac-shaped cell construct in which cells having the properties of trophectodermal cells are distributed on the outer circumference is unlikely to be obtained and is also unlikely to be induced to differentiate into endodermal cells. Hence, it is considered that a collection rate was decreased as shown in results of Comparative Example 1 mentioned later. When a cell-adhesive part has a round shape, it is considered that the cell-adhesive part had a large area which consumed a time for forming an accumulating portion.

A plurality of cell culture parts 20, if present, as in cell culture substrate 1 are isolated from each other and arranged apart from each other with a distance of preferably 0.20 mm or more and more preferably 0.30 mm or more. By arranging cell culture parts 20 to be apart from each other with not less than a certain distance, cells within each cell culture part 20 are uniformly cultured with a certain distance from each of other cell culture parts 20 without forming intercellular bonds with cells in other cell culture parts 20 adjacent thereto, thereby making it possible to construct an experimental system with high reproducibility.

Cell culture substrate 1 according to each of the embodiments shown in FIG. 1, FIG. 15A, FIG. 15B, FIG. 15C, FIG. 15D, and FIG. 26 has a structure where one or more cell culture parts 20 are present in first non-cell-adhesive part 10. An embodiment of a cell culture substrate having no first non-cell-adhesive part 10 will be separately described with reference to FIGS. 35 to 37.

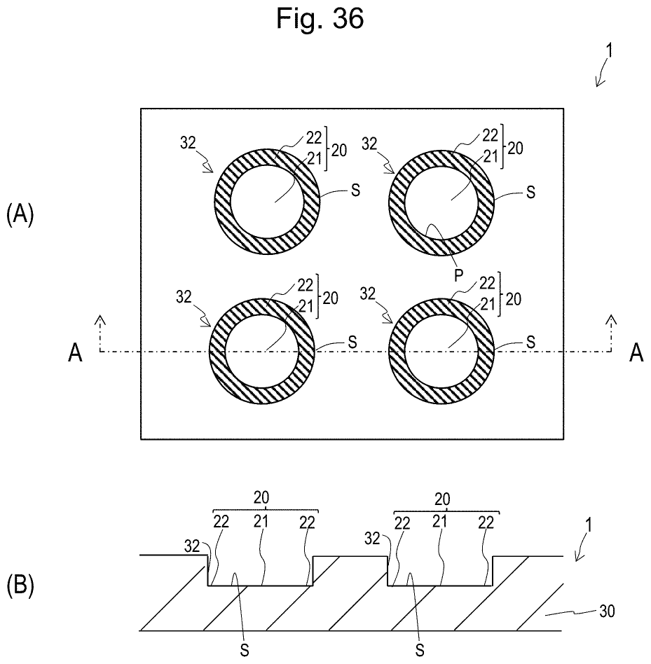

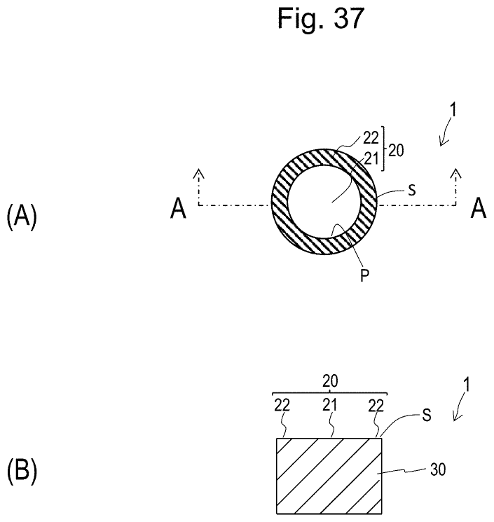

Differences of cell culture substrate 1 shown in each of FIGS. 35 to 37 from cell culture substrate 1 shown in FIG. 1 or FIG. 26 will be described below. In cell culture substrate 1 shown in each of FIGS. 35 to 37, features and production methods of non-cell-adhesive part (central part) 21 and cell-adhesive part 22 constituting cell culture part 20 are the same as in non-cell-adhesive part 21 and cell-adhesive part 22 in cell culture substrate 1 shown in FIG. 1 or FIG. 26, so that the description about cross-sectional features of non-cell-adhesive part 21 and cell-adhesive part 22 on the cross section of cell culture substrate 1 in FIG. 35(B), FIG. 36(B) or FIG. 37(B) is omitted. Other features that are not mentioned about cell culture substrate 1 shown in each of FIGS. 35 to 37 are the same as in cell culture substrate 1 shown in FIG. 1 or FIG. 26, so that the description is omitted.

Cell culture substrate 1 according to one embodiment of the present disclosure shown in FIG. 35 has surface S comprising cell culture part 20. The cell culture part 20 has non-cell-adhesive part 21, and cell-adhesive part 22 extending continuously or intermittently (FIG. 35 shows an example of extending continuously) along periphery P of the non-cell-adhesive part 21 and surrounding the non-cell-adhesive part 21. In the cell culture substrate 1 shown in FIG. 35, a portion the surface on which cell-adhesive part 20 is arranged is referred to as “support substrate 30”.

The cell culture substrate 1 shown in FIG. 35 has support substrate 30 having one or more protrusions 31, and comprises non-cell-adhesive part 21 and cell-adhesive part 22 surrounding the non-cell-adhesive part 21 on upper surface S of each protrusion 31. In this embodiment, the protrusion 31 has round upper surface S, though the protrusion 31 may have other shapes. In this embodiment, the cell-adhesive part 22 is present on the periphery of the upper surface S of the protrusion 31, and the support substrate is absent outside the cell-adhesive part 22, in a planar view. Therefore, cells that have adhered to the cell-adhesive part 22, when cultured, do not spread to the outside of the cell-adhesive part 22, and spread on the cell-adhesive part 22 and the non-cell-adhesive part 21 thereinside to form a cell construct.

According to the embodiment shown in FIG. 35, since cell culture parts 20 (each consisting of non-cell-adhesive part 21 and cell-adhesive part 22) are present on upper surfaces S of protrusions 31 isolated from each other in the horizontal direction, cells within each cell culture part 20 are cultured without forming intercellular bonds with cells in other cell culture parts 20 adjacent thereto, easily constructing an experimental system with high reproducibility. In this embodiment, a plurality of protrusions 31, if present, are isolated from each other and arranged apart from each other with a distance of preferably 0.20 mm or more and more preferably 0.30 mm or more.

Cell culture substrate 1 according to one embodiment of the present disclosure shown in FIG. 36 has surface S comprising cell culture part 20. The cell culture part 20 has non-cell-adhesive part 21, and cell-adhesive part 22 extending continuously or intermittently (FIG. 36 shows an example of extending continuously) along periphery P of the non-cell-adhesive part 21 and surrounding the non-cell-adhesive part 21. In the cell culture substrate 1 shown in FIG. 36, a portion the surface on which cell-adhesive part 20 is arranged is referred to as “support substrate 30”.

The cell culture substrate 1 shown in FIG. 36 has support substrate 30 having one or more depressions 32, and comprises non-cell-adhesive part 21 and cell-adhesive part 22 surrounding the non-cell-adhesive part 21 on bottom surface S of each depression 32. In this embodiment, the depression 32 has round bottom surface S, though the depression 32 may have other shapes. In this embodiment, the cell-adhesive part 22 is present on the periphery of the bottom surface S of the depression 32, and the outside of the cell-adhesive part 22 is the peripheral wall surface of the depression 32, in a planar view. Therefore, cells that have adhered to the cell-adhesive part 22, when cultured, do not spread to the outside of the cell-adhesive part 22, and spread on the cell-adhesive part 22 and the non-cell-adhesive part 21 thereinside to form a cell construct.

According to the embodiment shown in FIG. 36, since cell culture parts 20 (each consisting of non-cell-adhesive part 21 and cell-adhesive part 22) are present on bottom surfaces S of depressions 32 isolated from each other in the horizontal direction, cells within each cell culture part 20 are cultured without forming intercellular bonds with cells in other cell culture parts 20 adjacent thereto, easily constructing an experimental system with high reproducibility. In this embodiment, a plurality of depressions 32, if present, are isolated from each other and arranged apart from each other with a distance of preferably 0.20 mm or more and more preferably 0.30 mm or more.

Cell culture substrate 1 according to one embodiment of the present disclosure shown in FIG. 37 has surface S comprising cell culture part 20. The cell culture part 20 has non-cell-adhesive part 21, and cell-adhesive part 22 extending continuously or intermittently (FIG. 37 shows an example of extending continuously) along periphery P of the non-cell-adhesive part 21 and surrounding the non-cell-adhesive part 21. In the cell culture substrate 1 shown in FIG. 37, a portion the surface on which cell-adhesive part 20 is arranged is referred to as “support substrate 30”.

The cell culture substrate 1 shown in FIG. 37 has one cell culture part 20 formed throughout flat surface S of support substrate 30, and cell-adhesive part 22 is arranged on the periphery of the surface S. In this embodiment, the support substrate 30 has round surface S, though the support substrate 30 may have other shapes. In this embodiment, the cell-adhesive part 22 is present on the periphery of the surface S of the support substrate 30, and the support substrate is absent outside the cell-adhesive part 22, in a planar view. Therefore, cells that have adhered to the cell-adhesive part 22, when cultured, do not spread to the outside of the cell-adhesive part 22, and spread on the cell-adhesive part 22 and the non-cell-adhesive part 21 thereinside to form a cell construct.

According to the embodiment shown in FIG. 37, since only cell culture part 20 (consisting of non-cell-adhesive part 21 and cell-adhesive part 22) is present in one cell culture substrate 1, cells within the cell culture part 20 are cultured without forming intercellular bonds with other cells, easily constructing an experimental system with high reproducibility.

### <4. Cell Construct>

A cell construct produced according to an embodiment of the present disclosure comprises small intestinal epithelial cells.

The small intestinal epithelial cells are typically small intestinal epithelial cells expressing a trophectodermal cell marker.

An embodiment of the present disclosure is based on the surprising finding that cells accumulate with a high density in a cell-adhesive part extending so as to surround a non-cell-adhesive part during cell culture, and differentiate into small intestinal epithelial cells expressing a trophectodermal cell marker, to obtain a cell construct having a sac-shaped structure where the resulting cells are distributed on the outer circumference. In Examples, it has been confirmed that cytokeratin 7, a marker of trophectodermal cells, and CDX2, a marker of small intestinal epithelial cells and trophectodermal cells, were strongly expressed in cells that accumulated on the cell-adhesive part.

The small intestinal epithelial cells can be confirmed based on the expression of transcription factors CDX2 and HNF4 in cell nuclei and villin in villi and the expression of an endodermal marker E-cadherin, etc. The presence of these markers can be detected by immunohistochemical staining using antibodies, PCR evaluation using mRNA, or the like.

A cell construct according to an embodiment of the present disclosure comprises small intestinal epithelial cells and is useful as a gut organoid having gut-like functions. The term “gut organoid” refers to a cell construct (tissue) having functions (specifically, for example, a motile movement function, a mucus secretion function, and a substance absorption function) similar to those of an intestine of the origin organism of cells, and in particular, an intestine of a mammal such as a human or a human intestine. The cell construct according to an embodiment of the present disclosure is useful in the development of drugs for preventing or treating intestinal diseases or pathological studies on intestinal diseases.

The whole shape of the cell construct according to an embodiment of the present disclosure is not particularly limited but it is usually granular. The word “granular” also encompasses “spherical.”

As in Non Patent Literature 4, strong expression of CDX2 indicates that mouse ES cells differentiate into trophectodermal cells. In Examples of the present disclosure, it has been confirmed that a marker CDX2 or cytokeratin 7 was strongly expressed in an accumulating portion, also suggesting differentiation into cells having the properties of trophectodermal cells.

Non Patent Literature 5 states that a tissue having a sac-shaped structure containing CDX2-positive cells is obtained from human iPS cells. Accordingly, in the present disclosure, the possibility is suggested that in an accumulating portion having dense accumulations of cells on a cell-adhesive part, stem cells differentiate by culture into CDX2-positive cells, which also corresponds to the formation of a sac-shaped structure.

Besides, Non Patent Literatures 6 to 8 also disclose that ES cells or iPS cells have the ability to differentiate into trophectodermal cells.

As shown in these past findings and later in the present disclosure, decrease in the expression of Oct3/4 gene has been observed in cells that have accumulated on a cell-adhesive part. Therefore, in the present disclosure, it is predicted that stem cells attach and grow on a cell-adhesive part to form an accumulating portion where the stem cells differentiate into small intestinal epithelial cells expressing a trophectodermal cell marker.

In studies shown in Non Patent Literature 9, decreased expression of Oct gene indicates that mouse ES cells differentiate into trophectoderm. It is predicted that similar differentiation also occurs in a cell construct according to an embodiment of the present disclosure.

A feature of a cell construct induced according to an embodiment of the present disclosure is the expression of ABCG2 transporter at a level equivalent to or more than that of cDNA expression in the small intestine of an origin animal (human small intestine when the origin animal is a human). The expression of ABCG2 transporter is markedly high as compared with previously obtained gut organoids.

The ABCG2 transporter in the intestine contributes to excretion, for example, excretion of uric acid, in the intestine as a role, and its decreased function is considered to cause symptoms such as gout. Pharmacokinetic observation requires examining not only absorpition but excretion. In this context, cDNA of the small intestine of an origin animal and in particular, the human small intestine, which can be used as a reference of a relative value, is not particularly limited by site, sex, age, race, etc. The site is desirably the jejunum or the ileum which is the hindgut. The age desirably reaches the age of adulthood. Also, various commercially available products may be used, and obtained from a human tissue obtained after receiving informed consent. PCR Ready First Strand cDNA—Human Adult Normal Tissue: Small Intestine (BioChain Institute Inc.), or PCR Ready First Strand cDNA-Human Adult Normal Tissue: Ileum (BioChain Institute Inc.) which is a product obtained from the ileum in order to reliably obtain the expression of CUBN can be used as commercially available products of human small intestinal cDNA.

However, the expression of the ABCG2 transporter is not sufficient in previously induced gut organoids. Examples of a past analysis case include an example of Non Patent Literature 10 in which expression is observed only at a level equal to or less than that in a human intestine. In a gut organoid obtained using human ES cells as described in Non Patent Literature 3, the expression of the ABCG2 transporter is also at a level equal to or less than that in a human intestine. In the case of performing previous pattern culture described in Non Patent Literature 2 using iPS cells in Examples mentioned later, the obtained value has been equivalent to a value obtained using ES cells.

Non Patent Literatures 11 and 12 state that ABCG2 transporter is strongly expressed in the placenta, i.e., trophectodermal cells.

For example, Non Patent Literature 13 has reported that by culturing mouse intestinal tissues, organoid-like tissues are induced through ex vivo culture without the use of Lgr5-positive intestinal epithelial stem cells as used in Non Patent Literature 1. In this literature, Trop2-positive cells are expressed in a mouse embryonic intestine and serves as a basis for differentiation into an organoid. This Trop2 marker is also strongly expressed in trophectodermal cells which have similar properties. Hence, the mechanism of development into small intestinal epithelial cells according to an embodiment of the present disclosure is presumably similar thereto.

The similarity in properties between trophectodermal cells and small intestinal epithelial cells is also mentioned in Non Patent Literature 14.

Thus, in the present disclosure, it is suggested that a phenomenon described in Non Patent Literature 13 also occurs. It is considered that trophectodermal cells are induced to differentiate into small intestinal epithelial cells expressing a trophectodermal cell marker. Furthermore, owing to a short period of differentiation induction culture, a cell construct (tissue) is obtained in the state of early-phase cells. Therefore, it is considered that the expression level of ABCG2 transporter in the constituent cells is likely to be elevated.

In a cell construct according to an embodiment of the present disclosure, when an expression level of ABCG2 transporter and an expression level of ABCB1 transporter in the cell construct are indicated as relative values with respect to an expression level of ABCG2 transporter and an expression level of ABCB1 transporter, respectively, in a small intestinal tissue of an origin animal, the relative value of the expression level of ABCG2 transporter in the cell construct is 90 or more times and preferably 100 or more times the relative value of the expression level of ABCB1 transporter. The upper limit of the ratio is not particularly limited but it is, for example, 1000 or less times. The cell construct according to an embodiment of the present disclosure is useful as a model of an immature human intestine. For example, Non Patent Literature 15 describes the expression of ABCB1 transporter and ABCG2 transporter in the placenta or embryos. According to this description, the expression of ABCB1 transporter in the human embryonic small intestine is low up to 20 weeks post-implantation and is elevated in newborns to the same level as in adults. By contrast, the expression of ABCG2 transporter as high as that in adults is observed from 5.5 weeks post-implantation. Hence, in an immature intestine, the expression of ABCG2 transporter is considered to be higher than that of ABCB1 transporter.

In a case in which the origin animal of stem cells for use in the preparation of the cell construct is a human, human small intestinal cDNA that can be used as a reference is as already mentioned.

Non Patent Literature 16 describes the production of a gut organoid with higher expression of ABCG2 transporter than that in a human adult intestine by suspension culture. However, this product also has high expression of ABCB1 transporter by induction and thus differs from the one obtained according to an embodiment of the present disclosure. Furthermore, the product described in Non Patent Literature 16 consists only of small intestinal epithelial cells.

Another example of an approach of artificially elevating the expression of a transporter includes overexpression by gene transfer. However, there is no case in which the expression of a predetermined transporter is elevated in three-dimensional tissues of organoids, animal intestines, or the like, though this is achieved in cultured cells. In this respect as well, a cell construct according to an embodiment of the present disclosure has a feature.

Examples of an approach of measuring the intensity of expression of ABCG2 transporter and ABCB1 transporter include a method of measuring mRNA by a genetic approach (e.g., qPCR including real-time PCR, and comparison based on the density of a band obtained by agarose gel electrophoresis), and a method of measuring protein expression using an antibody (e.g., flow cytometry). A genetic approach of measuring a mRNA level is desirable.

In this context, the reason why the expression level of ABCG2 transporter is defined by its ratio to the expression level of ABCB1 transporter, not in itself, is as described below.

In a cell construct according to an embodiment of the present disclosure, the transporters are expressed mainly in epithelial cells but are also expressed in stromal cells. Hence, if the expression level of a protein (e.g., CDX2 or villin) expressed only in epithelial cells is used as a reference of the expression level of ABCG2 transporter, the value varies depending on the amount of stromal cells in the cell construct. This problem can be circumvented by evaluating the ratio between ABCG2 transporter and ABCB1 transporter, which are expressed together in cells.

A protein cubilin encoded by CUBN gene is a receptor that is rarely expressed in the duodenum and the jejunum but is expressed in the ileum, and works in the absorption of cobalamin (vitamin B12) (Jensen et al., Physiological Reports, e12086, 2014). A feature of a cell construct induced according to an embodiment of the present disclosure is a high expression level of CUBN. A cell construct comprising small intestinal epithelial cells and having a high expression level of CUBN is considered to have at least properties similar to those of the ileum. Since cubilin is expressed in small intestinal epithelial cells, it is preferable for evaluating the expression level of the CUBN gene to evaluate a relative value to the expression level of a marker characteristic of small intestinal epithelial cells. Examples of the marker characteristic of small intestinal epithelial cells can include CDX2, villin, and E cadherin. Villin is desirable.

Accordingly, a cell construct according to an embodiment of the present disclosure is preferably a cell construct comprising small intestinal epithelial cells, wherein

when an expression level of CUBN and an expression level of villin in the cell construct are indicated as relative values with respect to an expression level of CUBN and an expression level of villin, respectively, in a small intestinal tissue of an origin animal, the relative value of the expression level of CUBN in the cell construct is in a range of 0.2 to 1.8 and more preferably 0.24 to 1.72 with respect to the relative value of the expression level of villin.

A cell construct having this feature has at least properties similar to those of the ileum.

In a case in which the origin animal of stem cells for use in the preparation of the cell construct is a human, human small intestinal cDNA that can be used as a reference is as already mentioned.

A cell construct according to an embodiment of the present disclosure more preferably comprises endodermal cells, ectodermal cells and mesodermal cells.

The endoderm forms the digestive tract and tissues of organs, such as the lung, thyroid, pancreas and liver, and cells of secretory glands opening into the digestive tract, peritoneum, pleura, larynx, eustachian tube, trachea, bronchial tube, urinary tract (the bladder, most of the urethra, part of the ureter), and other tissues. Differentiation from ES cells or iPS cells into endodermal cells can be confirmed by measuring expression levels of endoderm-specific genes. Examples of endoderm-specific genes include AFP, SERPINA1, SST, ISL1, IPF1, IAPP, EOMES, HGF, ALBUMIN, PAX4, and TAT.

Particular endodermal cells that may be contained in a cell construct according to an embodiment of the present disclosure include small intestinal epithelial cells. The gut organoid preferably contains, as small intestinal epithelial cells, at least one type of cells selected from enterocytes, goblet cells, enteroendocrine cells, and Paneth cells, and particularly preferably contain, as small intestinal epithelial cells, all of enterocytes, goblet cells, enteroendocrine cells, and Paneth cells. It is possible to determine the presence of endodermal cells in a cell construct according to an embodiment of the present disclosure based on the positive expression of endodermal cell markers. The enterocyte marker may be CDX2, the goblet cell marker may be MUC2, the enteroendocrine cell marker may be CGA, and the Paneth cell marker may be DEFA6. In addition to the above, ECAD, Na⁺/K⁺-ATPase, and villin are intestinal epithelial cell markers. Further, it is also possible to use the definitive endoderm markers FOXA2, SOX17, and CXCR4 as markers for detecting endodermal cells. The primary endoderm and mesoderm markers GATA4, GATA6, and T (Brachyury) also can be used as markers for detecting endodermal cells.

The ectoderm forms several tissues including, for example, skin epidermis, epithelium of the urethral end of a man, hair, nails, skin glands (including mammary gland and sweat gland), sensory organs (oral cavity, pharynx, nose, epithelium at the distal end of the rectum, salivary gland) and lens. A part of the ectoderm is invaginated in a groove shape in the developmental process to form a neural tube, which is also a source of neurons and melanocytes of the central nervous system such as the brain and spinal cord. The ectoderm also forms the peripheral nervous system. Differentiation from ES cells or iPS cells into ectodermal cells can be confirmed by measuring expression levels of ectoderm-specific genes. Examples of ectoderm-specific genes may include β-TUBLIN, NESTIN, GALANIN, GCM1, GFAP, NEUROD1, OLIG2, SYNAPTPHYSIN, DESMIN, and TH.

Particular ectodermal cells that may be contained in a cell construct according to an embodiment of the present disclosure include cells that constitute the intestinal nerve plexus. It is possible to determine the presence of ectodermal cells in a cell construct according to an embodiment of the present disclosure based on the positive expression of ectodermal cell markers. As markers for detecting ectodermal cells, the intestinal nerve plexus marker PGP9.5 and the neural progenitor marker SOX1 can be used.

The mesoderm forms a body cavity and mesothelium lining inside thereof, muscles, skeletons, skin dermis, connective tissue, heart, blood vessels (including vascular endothelium), blood (including blood cells), lymph vessels, spleen, kidneys, ureters, and gonad (testis, uterus, and gonadal epithelium). Differentiation from ES cells or iPS cells into mesodermal cells can be confirmed by measuring expression levels of mesoderm-specific genes. Examples of mesoderm-specific genes may include FLK-1, COL2A1, FLT1, HBZ, MYF5, MYOD1, RUNX2, and PECAM1.

Particular mesodermal cells that may be contained in a cell construct according to an embodiment of the present disclosure include smooth muscle cells and interstitial cells of Cajal. It is possible to determine the presence of mesodermal cells in gut organoids based on the positive expression of mesodermal cell markers. As mesodermal cell markers, the smooth muscle cell marker a-smooth muscle actin (SMA) and the Cajal cell markers CD34 and CKIT (for double-positive cells) can be used. The primary endoderm and mesoderm markers GATA4, GATA6, and T (Brachyury) also can be used as markers for detecting mesodermal cells.

A cell construct according to an embodiment of the present disclosure preferably comprises small intestinal epithelial cells in at least a part of the outer surface thereof. According to this embodiment, substances outside of the cell construct can be absorbed into the inside via small intestinal epithelial cells on the outer surface thereof, which is preferable. In addition, in this embodiment, when small intestinal epithelial cells on the outer surface are transporter-positive, it allows uptake of substances via transporters, which is further preferable. In other words, the gut organoid containing transporter-positive small intestinal epithelial cells has gut-like substance absorption ability.

### <5. Method for Producing Cell Construct Comprising Small Intestinal Epithelial Cell>

A method for producing a cell construct comprising small intestinal epithelial cells according to an embodiment of the present disclosure comprises:

seeding stem cells onto a cell culture substrate having the features described above; and

culturing the stem cells seeded to differentiate a part of the stem cells into small intestinal epithelial cells.

The small intestinal epithelial cells are typically small intestinal epithelial cells expressing a trophectodermal cell marker.

A cell culture substrate used in an embodiment of the present disclosure is preferably subjected to precoating treatment with a precoating agent in order to promote adhesion of stem cells to cell-adhesive parts. A precoating treatment agent is a component for promoting adhesion of stem cells to cell-adhesive parts when applied in advance to a cell culture substrate. Examples of the precoating treatment agent include extracellular matrixes (collagen, fibronectin, proteoglycan, laminin, and vitronectin), gelatin, lysine, peptides, and gel matrixes containing any thereof, serum, and other substances. By performing precoating treatment, it is possible to promote adhesion of stem cells having low adhesiveness to cell-adhesive parts and effectively carry out adhesion culture and differentiation induction of cells.

Stem cells can be cultured under conditions that allow them to remain undifferentiated before seeding. A medium for use in this culture is not particularly limited as long as it is a medium that does not allow differentiation induction of stem cells. Examples thereof include a medium containing a leukemia inhibitory factor which are known to have a feature of allowing mouse embryonic stem cells and mouse induced pluripotent stem cells to remain undifferentiated and a medium containing basic FGF which are known to have a feature of allowing human iPS cells to remain undifferentiated.

The step of culturing the stem cells seeded in the cell culture substrate to differentiate a part of them into small intestinal epithelial cells can be performed in a medium that permits growth and differentiation induction of stem cells. The medium is not particularly limited. Specific examples thereof include media used in Patent Literature 1, Patent Literature 2, and Non Patent Literature 4, and commercially available media such as StemFit (Ajinomoto Co., Inc.), StemFlex (Life Technologies Corp.), and ReproFF (ReproCELL Inc.). The medium may be a serum-containing medium, or may be a serum-free medium containing a known component having a property of replacing serum. The contribution of factors (FGF2, IGF-1, and heregulin) included in Patent Literature 1, Patent Literature 2, and Non Patent Literature 4 to the growth of pluripotent stem cells is described in Non Patent Literatures 17 and 18.

As a medium, MEM medium, BME medium, DMEM medium, DMEM-F12 medium, αMEM medium, IMDM medium, ES medium, DM-160 medium, Fisher medium, F12 medium, WE medium and RPMI1640 medium, etc. can be used. It is possible to add various growth factors, antibiotics, amino acids, and other additives to a medium. For example, 0.1 to 2% pyruvic acid, 0.1 to 2% nonessential amino acids, 0.1 to 2% penicillin/streptomycin, 0.1 to 1% glutamine, 0.1 to 2% β mercaptoethanol, and a 1 mM to 20 mM ROCK inhibitor (e.g., Y-27632) may be added.

The seeding density of stem cells on a cell culture substrate is not particularly limited as long as it complies with an ordinary method. In an embodiment of the present disclosure, stem cells are seeded on a cell culture substrate at a density of preferably 3×10⁴ cells/cm² or more, more preferably 3×10⁴ to 5×10⁵ cells/cm², and further preferably 3×10⁴ to 2.5×10⁵ cells/cm².

The culture temperature is usually 37° C. It is preferable to carry out the culture under a CO₂ concentration atmosphere of about 5% by using an appropriate cell culture apparatus such as a CO₂ cell culture apparatus.

The culture period of stem cells after seeding to the cell culture substrate differs depending on the initial seeding density of cells and the shape and size of a cell-adhesive part but is preferably on the order of 2 to 4 weeks. The present inventors have found that: when stem cells are cultured on a cell culture substrate having a structure described herein and induced to differentiate, a cell construct comprising small intestinal epithelial cells induced to differentiate are detached by spontaneous floating in 2 to 4 weeks after seeding and can be collected; and the collection rate of the cell construct thus collected is markedly high. On a substrate having a round cell-adhesive part described in Non Patent Literature 3, a cell construct is detached after a lapse of 30 days or more, and furthermore, its collection rate is very low. In contrast to this, a method according to an embodiment of the present disclosure is advantageous.

Although a cell construct induced to differentiate by a method according to an embodiment of the present disclosure is detached by spontaneous floating from a cell culture substrate, the detachment of the cell construct from the cell culture substrate may be promoted by use of various approaches, such as mild enzymatic treatment (e.g., Accutase and TrypLE), EDTA treatment, flowing of a liquid such as a medium, and physical detachment with a scraper, which do not disrupt the cell construct.

After detachment of the cell construct from the cell culture substrate, suspension culture may be further continued. The period of the suspension culture is not limited.

A cell construct according to an embodiment of the present disclosure having a feature of an immature human small intestine having a higher expression level of ABCG2 transporter than the expression level of ABCB1 transporter can be obtained by detachment and collection in 2 to 4 weeks after seeding.

### <6. Cell Construct-Holding Substrate>

An alternative aspect of the present disclosure relates to a cell construct-holding substrate comprising:

a cell culture substrate having a surface comprising a cell culture part, the cell culture part comprising a non-cell-adhesive part, and a cell-adhesive part extending continuously or intermittently along the periphery of the non-cell-adhesive part and surrounding the non-cell-adhesive part; and

a cell construct comprising small intestinal epithelial cells allowed to adhere onto the cell culture part.

The cell construct-holding substrate holds a cell construct comprising small intestinal epithelial cells on a substrate and facilitates the handleability of the cell construct comprising small intestinal epithelial cells for use in tests aimed at the development of drugs for preventing or treating intestinal diseases, or pathological studies on intestinal diseases. Also, the cell construct-holding substrate may be used as a scaffold in culturing viruses or microbes, such as norovirus, which grow in small intestinal tissues.

By further continuing the culture of the cell construct on the cell construct-holding substrate in a medium for culturing the cell construct comprising small intestinal epithelial cells as described above, it is also possible to maturate the cell construct comprising small intestinal epithelial cells and release and collect it from the cell culture substrate.

The growth properties of cells constituting the cell construct are higher in the state of the cell construct allowed to adhere to a cell culture substrate than in the cell construct floating in a medium. Therefore, a cell construct-holding substrate according to an embodiment of the present disclosure is suitable for this purpose.

The cell construct adheres to a cell culture substrate while cells spread. Hence, a cell construct-holding substrate according to an embodiment of the present disclosure is suitable as a scaffold for culturing viruses or microbes that grow in small intestinal tissues.

For the cell construct-holding substrate, it is preferable that the cell construct should be in contact with a buffer solution or a medium such that the cell construct can survive.

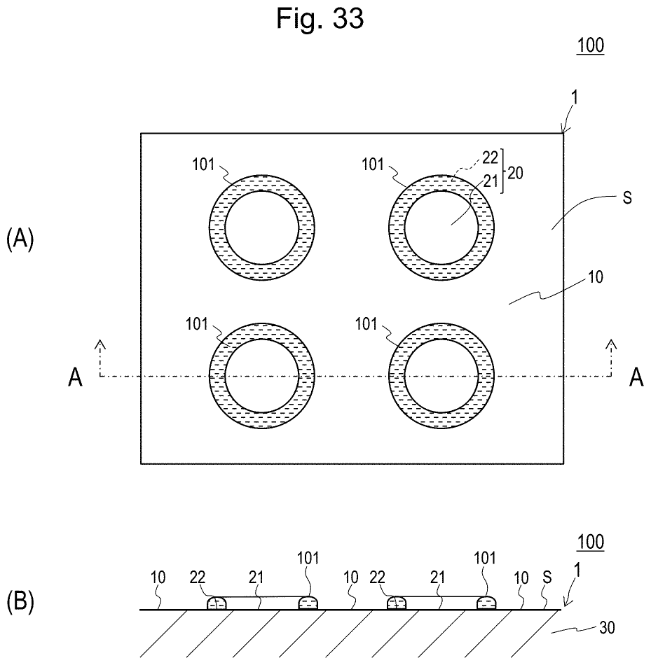

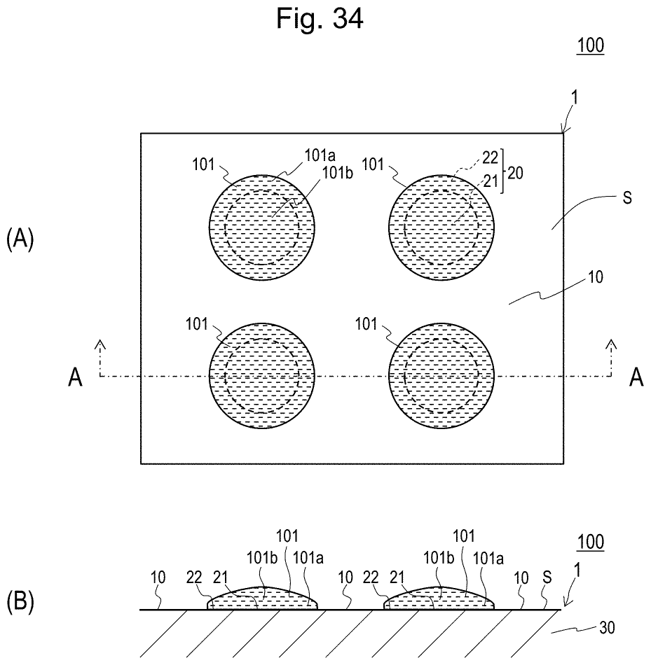

One embodiment of the cell construct-holding substrate will be described with reference to FIG. 33, and another embodiment thereof will be described with reference to FIG. 34.

Cell construct-holding substrate 100 according to the embodiment shown in FIG. 33 comprises cell culture substrate 1 and cell construct 101 comprising small intestinal epithelial cells.

The cell culture substrate 1 has surface S comprising cell culture part 20. The cell culture part 20 has non-cell-adhesive part (central part) 21, and cell-adhesive part 22 extending continuously or intermittently (FIG. 33 shows an example of extending continuously) along periphery P of the non-cell-adhesive part 21 and surrounding the non-cell-adhesive part 21. In the example shown in FIG. 33, the surface S further comprises first non-cell-adhesive part 10, and one or more cell culture parts 20 are arranged in the first non-cell-adhesive part 10. In the cell culture substrate 1, a portion in which first non-cell-adhesive part 10 and cell-adhesive part 20 are arranged on the surface is referred to as “support substrate 30”.

Features of the cell culture substrate and a production method thereof are as described herein and in particular, <2. Non-cell-adhesive part and cell-adhesive part of cell culture substrate> and <3. Feature of shape of cell culture part in cell culture substrate> described above, so that the description is omitted. Since the thicknesses of the first non-cell-adhesive part 10, the second non-cell-adhesive part (central part) 21, and the cell-adhesive part 22 and/or the level differences therebetween are sufficiently small with respect to the size of the cell construct 101, the surface S comprising the first non-cell-adhesive part 10, the second non-cell-adhesive part (central part) 21, and the cell-adhesive part 22 is shown as a flat plane surface in FIG. 33(B).

In the cell construct-holding substrate 100 according to the embodiment shown in FIG. 33, when the cell construct-holding substrate 100 is viewed along the normal direction of the surface S, the cell construct 101 comprising small intestinal epithelial cells is present at least at a position overlapping the cell-adhesive part 22. When stem cells are seeded to the cell culture substrate 1 where cell culture is performed, the stem cells attach and grow on the cell-adhesive part 22 to form an accumulating portion having dense accumulations of cells. Then, the cells of the accumulating portion differentiate into small intestinal epithelial cells expressing a trophectodermal cell marker in the formed accumulating portion of the cells to form cell construct 101 shown in FIG. 33. When the cell construct-holding substrate 100 is viewed along the normal direction of the surface S, the small intestinal epithelial cells present at a position overlapping the cell-adhesive part 22 are preferably cells expressing one or more markers selected from CDX2, cytokeratin 7 and E-cadherin.

Cell construct-holding substrate 100 according to the embodiment shown in FIG. 34 comprises cell culture substrate 1 and cell construct 101 comprising small intestinal epithelial cells. When the cell construct-holding substrate 100 is viewed along the normal direction of the surface S, the cell construct 101 comprising small intestinal epithelial cells is present at a position overlapping the cell-adhesive part 22 and the non-cell-adhesive part (central part) 21. In this embodiment, features of the cell culture substrate 1 are the same as in the embodiment shown in FIG. 33, so that the description is omitted. In FIG. 34(B) as well, the surface S comprising the first non-cell-adhesive part 10, the second non-cell-adhesive part (central part) 21, and the cell-adhesive part 22, of the support substrate 30 is shown as a flat plane surface.

When stem cells are seeded to the cell culture substrate 1 where cell culture is performed, the stem cells attach and grow on the cell-adhesive part 22 to form an accumulating portion having dense accumulations of cells. As shown in FIG. 33, the cell-adhesive part 22 is covered with the cell construct 101 comprising small intestinal epithelial cells. By further continuing culture, the non-cell-adhesive part (central part) 21 is covered with cells migrated from the cell construct 101 on the cell-adhesive part 22 to form cell construct 101 that covers the non-cell-adhesive part (central part) 21 and the cell-adhesive part 22.

When the cell construct-holding substrate 100 is viewed along the normal direction of the surface S, the cell construct 101 according to the embodiment shown in FIG. 34 preferably contains small intestinal epithelial cells in a larger amount at position 101a overlapping the cell-adhesive part 22 than at position 101b overlapping the non-cell-adhesive part (central part) 21. This can be confirmed by immunostaining and observing the cell construct 101 allowed to adhere on the cell culture substrate 1 using an antibody against a small intestinal epithelial cell marker (e.g., one or more markers selected from CDX2, cytokeratin 7 and E-cadherin). The cell construct-holding substrate 100 is viewed along the normal direction of the surface S. The mRNA level of a small intestinal epithelial cell maker in mRNA in a cell sample obtained from the position 101a overlapping the cell-adhesive part 22 in the cell construct 101 is compared with the mRNA level of the small intestinal epithelial cell marker in mRNA in a cell sample obtained from the position 101b overlapping the non-cell-adhesive part (central part) 21 in the cell construct 101. In a case in which the former is larger than the latter, it can be determined that the cell construct 101 contains small intestinal epithelial cells in a larger amount at the position 101a overlapping the cell-adhesive part 22 than at the position 101b overlapping the non-cell-adhesive part (central part) 21.

When the cell construct-holding substrate 100 is viewed along the normal direction of the surface S, the cell construct 101 according to the embodiment shown in FIG. 34 preferably contains nerve cells and muscle cells at position 101b overlapping the non-cell-adhesive part (central part) 21 and more preferably contains nerve cells and muscle cells in a larger amount at position 101b overlapping the non-cell-adhesive part (central part) 21 than at position 101a overlapping the cell-adhesive part 22.

In another preferable example of the cell construct 101 according to the embodiment shown in FIG. 34, it preferably comprises endodermal cells (including small intestinal epithelial cells), ectodermal cells and mesodermal cells, preferably has a sac-shaped structure, and further preferably has the features described in <4. Cell construct> described above.

A cell construct-holding substrate according to one or more embodiments of the present disclosure can be produced by seeding stem cells on the surface of a cell culture substrate and culturing them. Preferable embodiments of the stem cells and culture conditions are as described in <1. Stem cell> and <5. Method for producing cell construct comprising small intestinal epithelial cell> described above. However, for the purpose of producing a cell construct-holding substrate, it is preferable that a cell culture substrate after reaching a desired phase before a cell construct that has adhered onto a cell culture part of the cell culture substrate is released from the cell culture substrate should be exposed to conditions under which the culture and maturation of the cell construct are unlikely to proceed, to prepare a cell construct-holding substrate product.

Examples of the “conditions under which the culture and maturation of the cell construct are unlikely to proceed” include low-temperature conditions, and conditions in media or buffer solutions containing no nutrient factor or containing a low concentration of a nutrient factor.

Hereinafter, the present disclosure will be described with reference to specific experimental results. However, the scope of the present disclosure is not limited by the experimental results.

### EXAMPLES

### Example 1

### (Production of Cell Culture Substrate)

A cell culture substrate having a cell-adhesive part made of an annular pattern having an inside diameter of 180 μm, 280 μm or 380 μm and a width of 60 μm (see FIG. 1), which was a region formed by the oxidation decomposition of a layer of polyethylene glycol 400 formed on a glass substrate, and a non-cell-adhesive part which was a region of the surface (inside and outside the annular pattern of the cell-adhesive part) of the glass substrate coated with polyethylene glycol 400 was produced. The cell culture substrate has a plurality of cell-adhesive parts formed at intervals of 300 to 500 μm and made of the annular pattern (see FIG. 1). In the description below, the cell-adhesive part made of the annular pattern is referred to as “annular cell-adhesive part”.

The cell culture substrate was produced by the procedures described in JP Patent No. 5070565 (Patent Literature 6) and Okochi et al., Langmuir, Vol. 25, p. 6947-6953, 2009 (Non Patent Literature 19). The summary thereof will be described below.

### (First Stage Reaction)

39.0 g toluene, 0.48 g epoxysilane TSL8350 (manufactured by GE Toshiba Silicones Co., Ltd.) , and 0.97 g triethylamine were mixed and stirred at room temperature for 10 minutes. A 10-cm square glass substrate, which was previously UV-cleaned, was immersed in the silane solution such that its surface to be washed turned up. After being left at room temperature for 16 hours, the substrate was washed with ethanol and water and dried in nitrogen blow. A thin film containing an epoxy group was thereby formed on the glass substrate surface.

### (Second Stage Reaction)

Polyethylene glycol having an average molecular weight of 400 (PEG400) in an amount of 50 g was stirred, during which concentrated sulfuric acid was added dropwise in an amount of 25 μl. The resulting mixture was stirred as it was for several minutes, and the whole amount was then transferred to a glass dish. The above-described substrate was immersed therein so as to allow reaction to proceed at 80° C. for 20 minutes. After the reaction, the substrate was washed well with water and dried in nitrogen blow. A uniform hydrophilic thin film was thereby formed on the glass surface.

### (Oxidation Treatment)

A photomask having a titanium oxide photocatalyst applied on the whole surface was produced. The photomask used was in a 5-inch size and had openings with shapes corresponding to the plurality of annular cell-adhesive parts with the above-described size at intervals of 300 to 500 μm. The photomask also had openings having a width of about 1.5 cm located therearound. The illuminance of a light exposure machine was measured in advance at a wavelength of 350 nm and used as a guideline for the setting of light exposure time. The photocatalyst layer of this photomask was contacted with the hydrophilic thin film of the substrate surface, and the resultant was loaded in the light exposure machine so as to be irradiated with light from the photomask side. The hydrophilic thin film of the substrate surface was partially oxidation-decomposed by light exposure for 50 seconds with a mercury lamp having an illuminance of 20 mW/cm² at a wavelength of 350 nm. This substrate was cut into a size of 25 mm×15 mm and used as a cell culture substrate. The cell culture substrate was subjected to EOG sterilization treatment for 22 hours before being used in cell culture.

The cell culture substrate was disposed on the bottom surface of a 3.5 cm petri dish (Corning Inc.), coated by contact with vitronectin (Life Technologies Corp.) diluted 1/100 with phosphate-buffered saline (PBS) at room temperature for 30 minutes or longer, then washed with PBS three times, and then used.

The cell culture substrate thus obtained has a cross-sectional structure as shown in FIG. 1(B).

### (Culture)

The Department of Cell Engineering, Department of Reproductive Biology of NCCHD established Edom iPS cells as a human iPS cell line by causing cells obtained from menstrual blood to transiently express four Yamanaka factors with Sendai virus vectors (PLoS Genet. 2011 May; 7 (5): e1002085. Published online 2011 May 26. doi: 10.1371/journal.pgen.1002085PMCID: PMC3102737). Edom iPS cells were allowed to grow in advance using StemFit medium (Ajinomoto Co., Inc.) in a vitronectin-coated dish for cell culture (Corning Inc.). The cells thus allowed to grow were detached from the dish by treatment with EDTA (Invitrogen Corp.) diluted 1/1000 with PBS at 37° C. for 10 minutes, then seeded at 1×10⁶ cells to the cell culture substrate, and cultured. As a medium, the XF hESC medium described in Non Patent Literature 3 was used. On the day of seeding, the medium contained Y27632 but was replaced with the medium free from Y27632 on the following day and maintained. On day 4 or later, medium replacement was performed once every 3 to 4 days. During culture, tissues spontaneously detached from the cell culture substrate were collected, and maintained by suspension culture using the same medium as above in another petri dish.

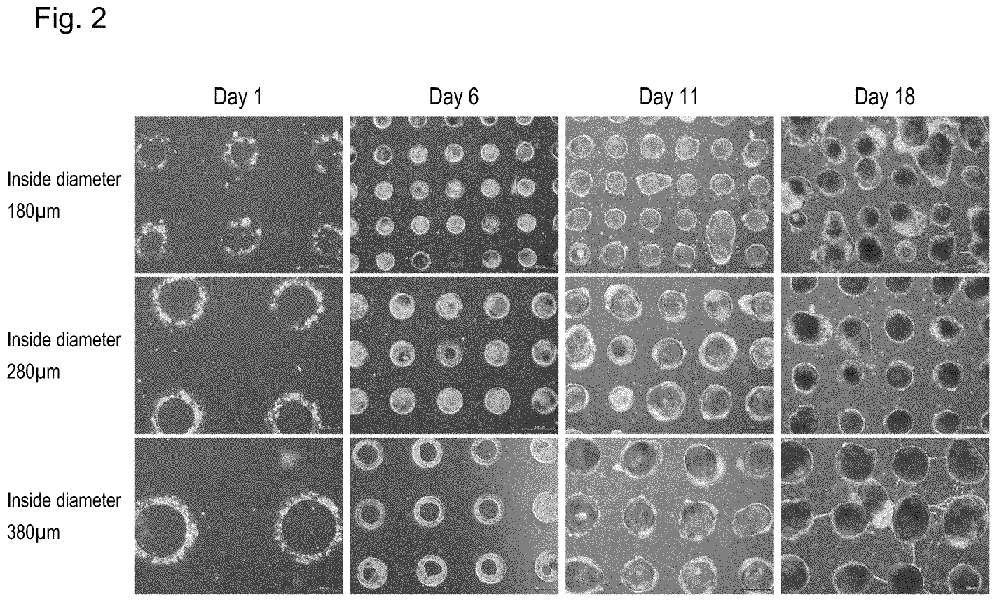

FIG. 2 shows observation images of cultures on culture days 1, 6, 11 and 18 obtained by culture using each cell culture substrate having an annular cell-adhesive part having a different inside diameter. The photographs of culture day 1 have a higher magnification factor than that of the others. As observed, cells grew first in the annular cell-adhesive part, and then, the inside non-cell-adhesive part surrounded by the annular cell-adhesive part was covered with the cells that grew.

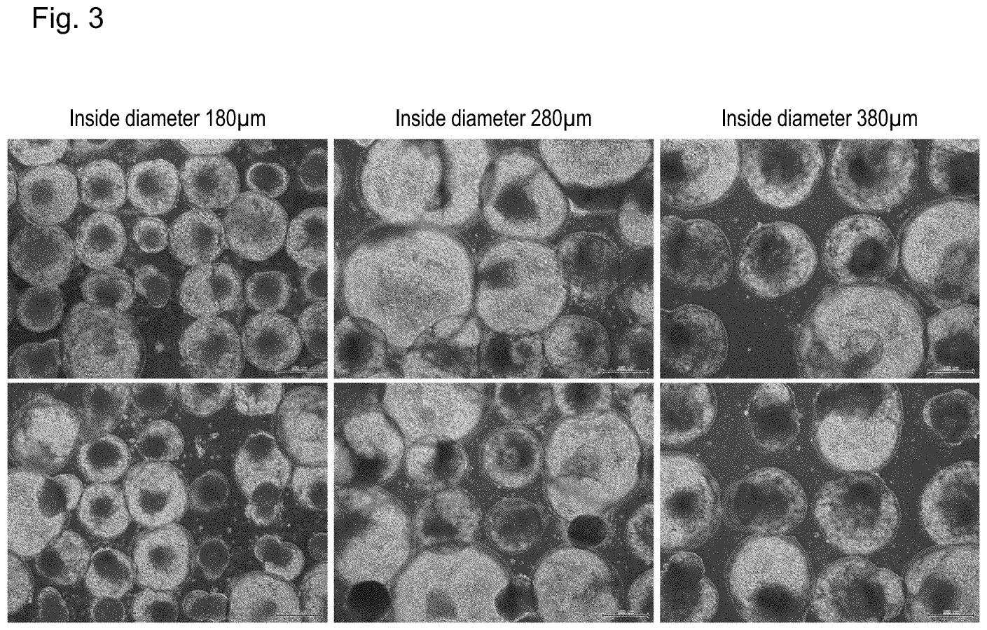

FIG. 3 shows observation images of cultures on culture week 3. The observation images in FIG. 3 show that tissues having a sac-shaped structure were formed at a high proportion by culture on each cell culture substrate.

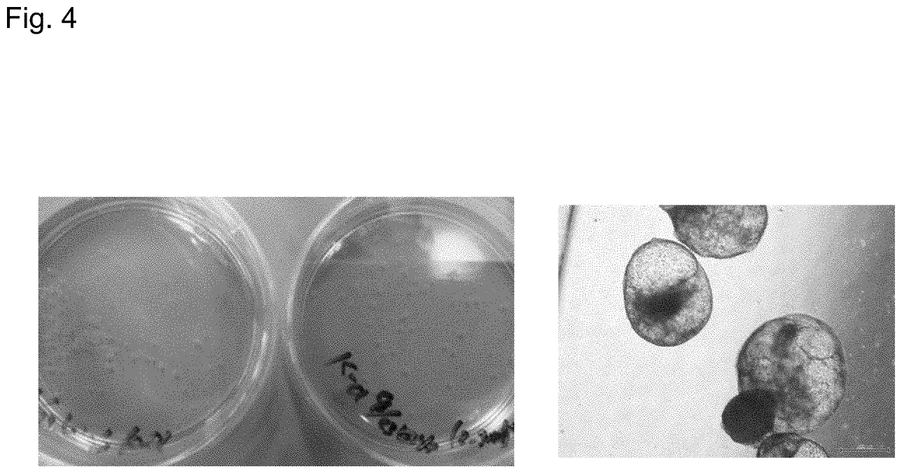

The results of this culture showed that tissues having a sac-shaped structure are spontaneously detached from the surface in 2 to 3 weeks after the start of culture and can be collected. FIG. 4 shows observation images of a tissue having a sac-shaped structure formed on and detached from a substrate having an annular cell-adhesive part having an inside diameter of 380 μm (the left photograph shows the whole dish, and the right photograph is an observation image of the tissue). In the left photograph of FIG. 4, white small objects seen in the dish are tissues having a sac-shaped structure. The right photograph of FIG. 4 is a microscopic observation image of tissues having a sac-shaped structure obtained in this Example. The observation image of tissues having a sac-shaped structure obtained in this Example is similar to an observation image of sac-shaped tissues (gut organoids) having intestinal functions obtained by culturing iPS cells in a medium similar to that used herein, as described in Uchida et al., JCI Insight, Vol. 2, e86492 2017. From this and results of Example 4 and Example 5 mentioned later, it is evident that the tissues having a sac-shaped structure obtained in this Example are also gut organoids.

The proportion of the collected tissues having a sac-shaped structure to the number of annular cell-adhesive parts on the cell culture substrate (hereinafter, also referred to as “tissue collection rate”) was 80% or more and was thus high yields.

### Comparative Examples 1 to 3

In Comparative Example 1, the cell culture substrate described in Uchida et al., JCI Insight, Vol. 2, e86492 2017 was prepared by forming a non-cell-adhesive part which was a region coated with polyethylene glycol layer, and a plurality of round cell-adhesive parts of 1500 μm in diameter formed by the oxidation decomposition of the polyethylene glycol layer.

In Comparative Example 2, a cell culture substrate having the same structure as in Comparative Example 1 was prepared except that the respective diameters of the plurality of round cell-adhesive parts were 282 μm.

The methods for producing the cell culture substrates of Comparative Example 1 and Comparative Example 2 were similar to the method for producing the cell culture substrate of Example 1, and the shape of openings in a photomask can be appropriately changed according to the cell-adhesive parts.

In Comparative Example 3, a glass substrate provided with no adhesive pattern was prepared.

The seeding and culturing of Edom iPS cells were performed on the substrate of each of Comparative Examples 1 to 3 under the same conditions as in Example 1.

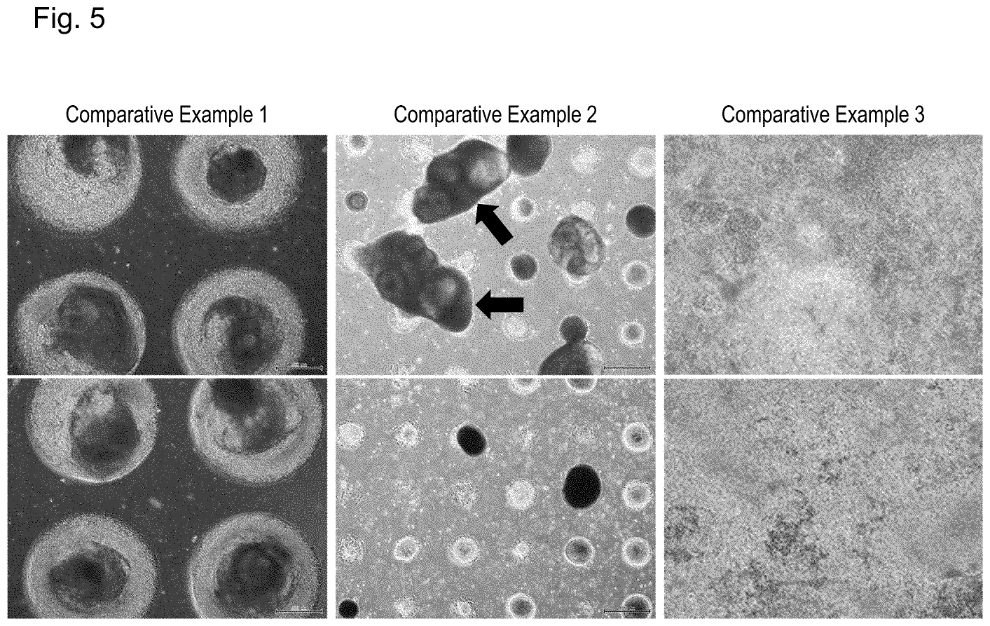

FIG. 5 shows microscopic observation images of cultures on 3 weeks after the start of culture on the substrate in each of Comparative Example 1, Comparative Example 2, and Comparative Example 3.

In Comparative Example 1, the start of detachment of tissues having a sac-shaped structure from the surface was observed 3 to 4 weeks after the start of culture, and the tissue collection rate was 4 to 5%. Comparative Example 1 compared with Example 1 described above had a long culture period until detachment and a low tissue collection rate.

In Comparative Example 2, tissues having a sac-shaped structure were obtained 2 to 3 weeks after the start of culture, whereas many cell aggregates which were dark in the observation images were obtained. The tissue collection rate of the tissues having a sac-shaped structure was 10% or less. As in the tissues indicated by arrows in “Comparative Example 2” of FIG. 5, tissues having a large-sized sac-shaped structure formed by the fusion of cultures were also formed in a plurality of adjacent round patterns.

In Comparative Example 3, there was a case in which about one or two small sac-shaped structures were obtained, whereas cell growth on the whole surface of the substrate was required before tissues having a sac-shaped structure were formed. Besides, a time of one month or more was required for culture, and the number of formed tissues having a sac-shaped structure was much smaller than that in the case of Example 1. Since the number of patterns in the cell-adhesive parts is not defined, the tissue collection rate cannot be calculated in this case.

These results show that the culture of iPS cells in a substrate having a plurality of cell-adhesive parts made of an annular pattern, as in Example 1, can yield tissues having a sac-shaped structure in a short culture period, and offers a markedly high tissue collection rate.

### Example 2

In order to perform the follow-up of the pattern culture in Example 1, the following time lapse observation was carried out.

Equipment was produced in which the cell culture substrate having a plurality of annular cell-adhesive parts having an inside diameter of 380 μm and a width of 60 μm, used in Example 1, was mounted on the bottom surface of a dish. By using this equipment, the seeding and culturing of Edom iPS cells were performed under the same conditions as in Example 1.

Photographs were taken using BioStation (Nikon Corp.) every 12 hours from culture days 4 to 21. The photographing operation followed the attached manual, and medium replacement was performed once every 2 to 3 days.

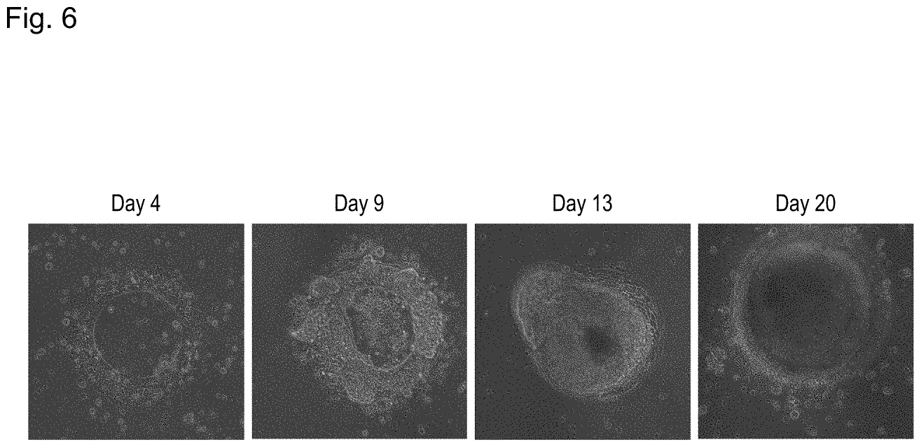

FIG. 6 shows observation images of cells around one annular cell-adhesive part on each of culture days 4, 9, 13 and 20. From this, it was confirmed that cells first grew and formed a layer on the annular pattern and then formed a sac-shaped structure up to culture day 21.

### Example 3

The formation of tissues having a sac-shaped structure was attempted using other cell types.

SEES2 cell line of human ES cells was cultured using the same cell culture substrate having annular cell-adhesive parts having an inside diameter of 180 μm, 280 μm or 380 μm and a width of 60 μm as in Example 1. First, growing culture was performed in a vitronectin-coated dish for cell culture (Corning Inc.) using StemFit medium (Ajinomoto Co., Inc.) supplemented with rhLIF (Wako Pure Chemical Industries, Ltd.) diluted 1/1000. The cells growing-cultured were collected by detachment treatment with Accutase (Life Technologies Corp.) at 37° C. for 5 minutes, then seeded to the substrate, and cultured by the same procedures as in Example 1.

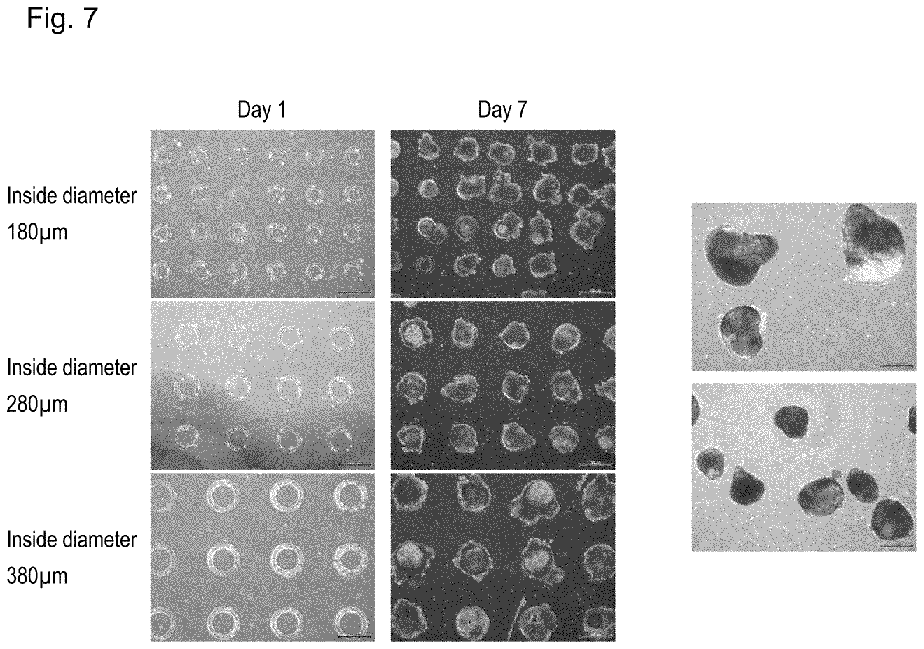

FIG. 7 shows observation images on days 1 and 7 of culture using each cell culture substrate, and observation images of tissues having a sac-shaped structure collected after 3-week culture using the cell culture substrate having annular cell-adhesive parts having an inside diameter of 280 μm. The observation images and tissue collection rate of the tissues having a sac-shaped structure were similar to those of Example 1 using iPS cells.

These results show that: the culture of stem cells on a cell culture substrate comprising an annular cell-adhesive part can efficiently produce tissues having a sac-shaped structure; and the formation of such tissues also takes place in the case of using ES cells in the same manner as in the case of using iPS cells in Example 1, irrespective of the type of pluripotent stem cells.

### Example 4

In order to study the process of culture in Example 1, cell culture was performed on the same cell culture substrate having annular cell-adhesive parts having an inside diameter of 380 μm and a width of 60 μm as in Example 1, and marker expression was examined by immunostaining.

The cell culture substrate having annular cell-adhesive parts having an inside diameter of 380 μm and a width of 60 μm, and cell culture conditions are as described in Example 1.

The cell culture substrate containing tissues on culture day 4, 7 or 12 to 14 was fixed in 4% paraformaldehyde (Wako Pure Chemical Industries, Ltd.) at room temperature for 20 minutes, then washed with PBS, and subjected to blocking operation with PBS containing 1% BSA and 0.1% Triton at room temperature for 30 minutes. Thereafter, the substrate was incubated at room temperature for 1 hour with a mouse IgG1-labeled anti-cytokeratin 7 antibody (Abcam plc, dilution ratio: 1/500), a mouse IgG3b-labeled anti-Oct3/4 antibody (Santa Cruz Biotechnologies, Inc., dilution ratio: 1/200), a rabbit IgG-labeled anti-Ki67 antibody (Abcam plc, dilution ratio: 1/500) or a rabbit IgG-labeled anti-CDX2 antibody (Abcam plc, dilution ratio: 1/1000). The substrate thus incubated was washed with PBS three times and then incubated at room temperature for 30 minutes with an Alexa 488-labeled anti-rabbit IgG antibody (Molecular Probes, dilution ratio: 1/1000) or an Alexa 546-labeled anti-mouse IgG antibody (Molecular Probes, dilution ratio: 1/1000) diluted with PBS. The substrate thus incubated was further washed with PBS three times. Thereafter, the nuclei of cells on the substrate were stained with DAPI (Sigma-Aldrich Co. LLC, dilution ratio: 1/1000) at room temperature for 10 minutes, then mounted, and observed under a confocal microscope. The types of the antibodies were appropriately chosen.

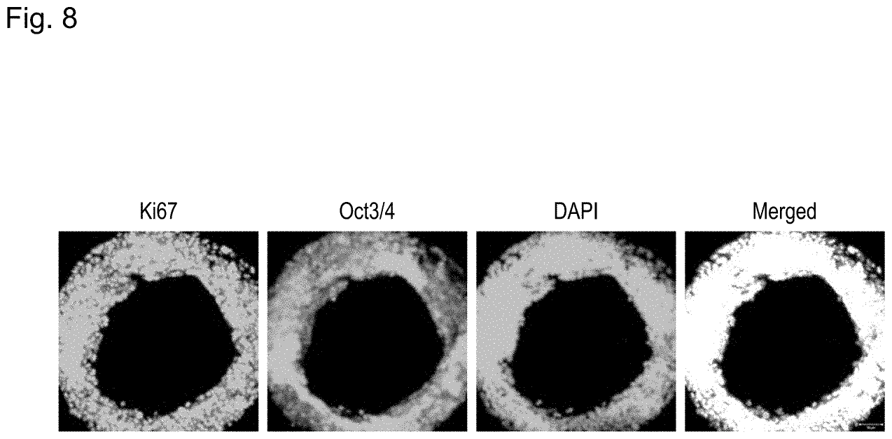

FIG. 8 shows observation images on culture day 4. As a result, Ki67-positive and Oct3/4-positive undifferentiated cells having the ability to grow were present on the annular cell-adhesive part on culture day 4, suggesting that an accumulating portion can be formed.

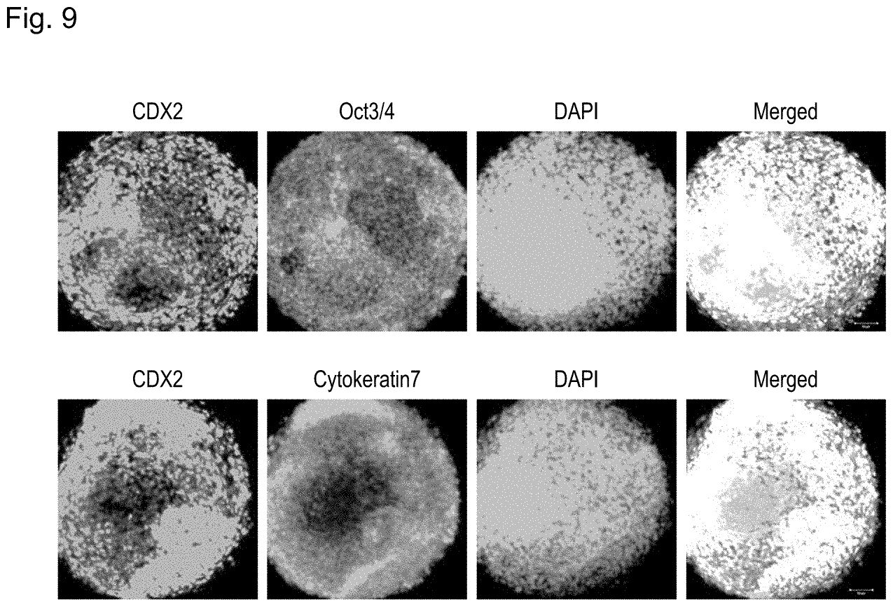

FIG. 9 shows observation images on culture day 7. The upper observation images of FIG. 9 show that tissues were formed in which Oct3/4-positive undifferentiated cells having multilineage potential were present mainly in the inside and CDX2-positive cells were present in the outer circumference. The lower observation images of FIG. 9 show that the CDX2-positive cells were cytokeratin 7-positive trophectodermal cells.

These results suggested that in tissues formed by culturing pluripotent stem cells on the cell culture substrate having annular cell-adhesive parts of Example 1, a particularly dense portion (an accumulating portion) formed by cells in the outer circumference consisted of trophectodermal cells and then, undifferentiated cells migrated into the inside through growth.

A cell culture substrate having annular cell-adhesive parts having an inside diameter of 600 μm and a width of 100 μm was further produced under the conditions described in Example 1. Edom iPS cells were cultured on this cell culture substrate under the conditions described in Example 1. On culture day 9, cell constructs that adhered to the cell-adhesive parts of the cell culture substrate were fixed, stained by the procedures described above using a mouse IgG2a-labeled anti-E-cadherin antibody for the endodermal cell marker (BD Pharmingen, dilution ratio: 1/1000), a rabbit IgG-labeled anti-βIII tubulin antibody for the nerve cell marker (Abcam plc, dilution ratio: 1/1000) or a mouse IgG2a-labeled anti-smooth muscle actin antibody for the muscle cell marker (SMA; Sigma-Aldrich Co. LLC, dilution ratio: 1/500) and each secondary antibody described above, and observed under a confocal microscope BZ-X710 (Keyence Corp.).

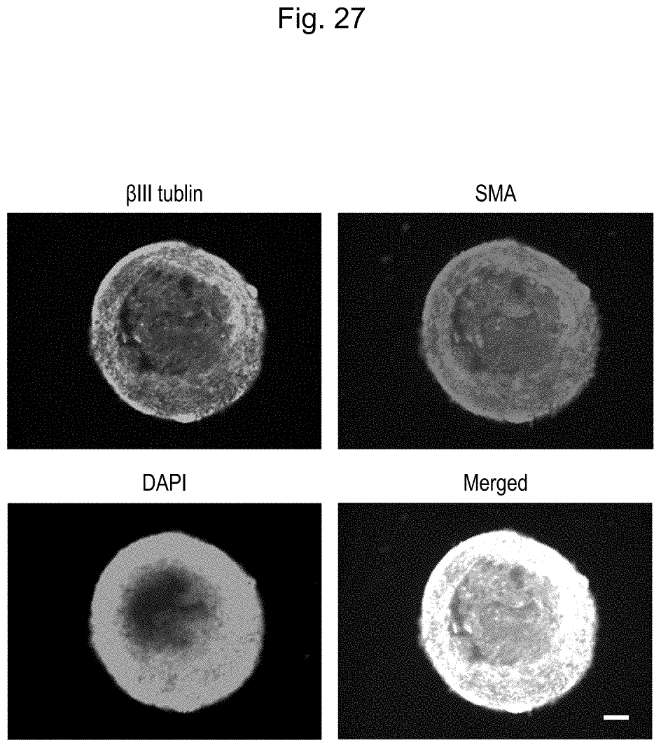

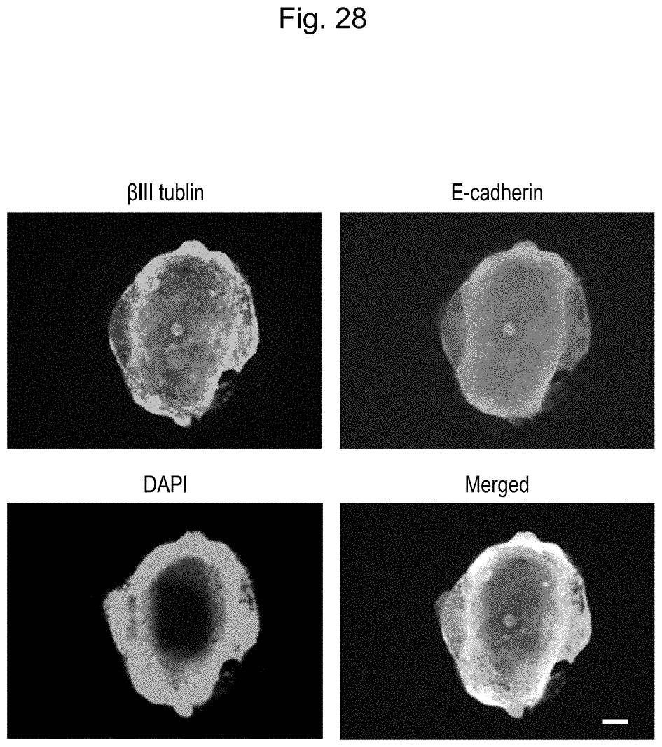

The results are shown in FIG. 27 and FIG. 28. From FIG. 27 and FIG. 28, it was confirmed that: a sac-shaped cell construct containing E-cadherin-positive cells was induced in the outer circumference; and the inside thereof (central part (non-cell-adhesive part) of an annular pattern) was occupied by migrated nerve cells or muscle cells. From this, it is considered that E-cadherin-positive cells constituting the outer circumference of the sac-shaped cell construct were induced on the annular cell-adhesive parts and the non-cell-adhesive central parts were covered with migrated nerve cells, muscle cells, or the like. The sac-shaped cell construct shown in FIG. 28 shrank as compared with the outer outline size of the annular cell-adhesive part. This shrinkage is probably influenced by cell migration, etc.

### Example 5

In order to study the presence or absence of intestinal cells and cells derived from 3 germ layers in tissues in Example 1, tissues having a sac-shaped structure obtained by culturing cells on a cell culture substrate having annular cell-adhesive parts having an inside diameter of 280 μm or an inside diameter of 380 μm and a width of 60 μm, collecting tissues spontaneously detached therefrom in 3 to 4 weeks after the start of culture, and suspension-culturing the tissues up to 6 weeks after the start of culture in another dish were evaluated by immunostaining.

The tissues were fixed overnight using iPGell (GenoStaff Co., Ltd.) and a 4% paraformaldehyde solution (Wako Pure Chemical Industries, Ltd.) according to the protocol attached to the product. The fixed tissues were embedded in paraffin, and then, tissue sections having a thickness of 4 to 6 μm were produced. Antibody staining was performed by the method described in Uchida et al., JCI Insight, Vol. 2, e86492, 2017. The antibody staining method is as described below.

The tissue sections were incubated overnight at 4° C. with a rabbit IgG-labeled anti-CDX2 antibody (Abcam plc; dilution ratio: 1/1000), a mouse IgG-labeled anti-villin antibody (Santa Cruz Biotechnologies, Inc.; dilution ratio: 1/200), a mouse IgG-labeled anti-smooth muscle actin antibody (Sigma-Aldrich Co. LLC; dilution ratio: 1/500), or a rabbit IgG-labeled anti-PGP9.5 antibody (DAKO; dilution ratio: 1/200) for primary antibody staining. The tissue sections thus stained with the primary antibody were washed with PBS three times for 5 minutes and then incubated at room temperature for 1 hour with an Alexa 488-labeled anti-rabbit IgG antibody or an Alexa 546-labeled anti-mouse IgG antibody (both from Molecular Probes; dilution ratio: 1/1000) diluted with PBS, for secondary antibody staining. The tissue sections thus stained with the secondary antibody were washed with PBS three times for 5 minutes. Thereafter, the nuclei of the cells were stained (DAPI; Sigma-Aldrich Co. LLC; dilution ratio: 1/1000) and mounted.

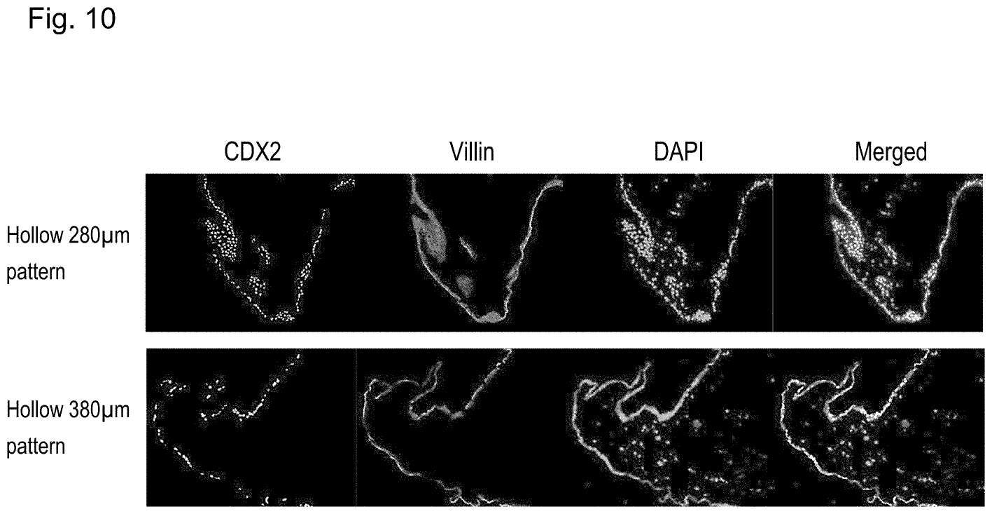

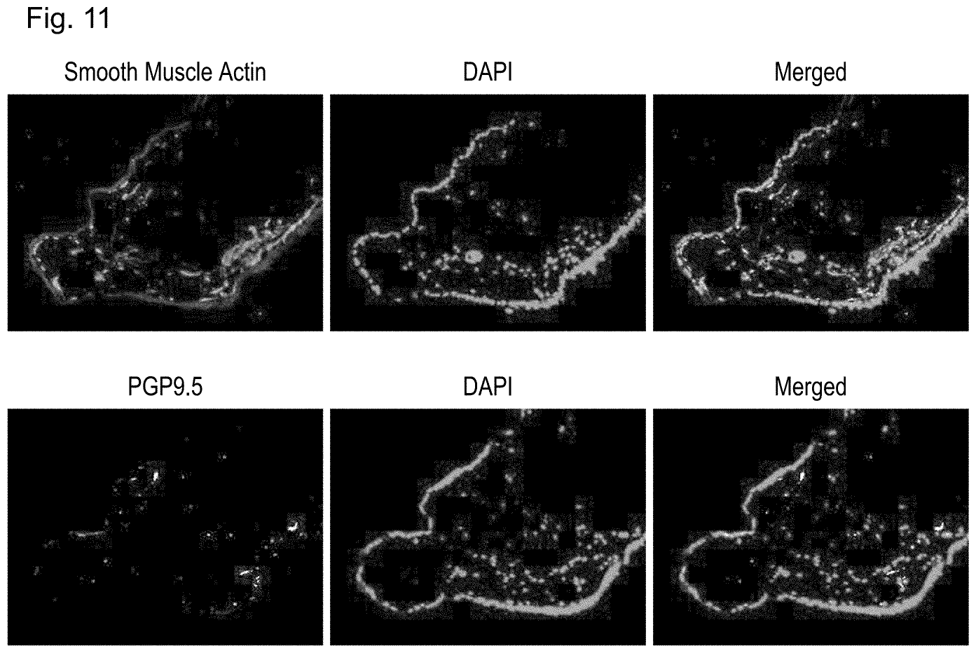

FIG. 10 shows results of staining, with an anti-CDX2 antibody, an anti-villin antibody and DAPI, of tissues formed by culture on the cell culture substrate having annular cell-adhesive parts having an inside diameter of 280 μm or 380 μm and a width of 60 μm in Example 1. The results shown in FIG. 10 show that the tissues formed in Example 1 included intestinal epithelial tissues having CDX2-positive cell nuclei and villin-positive epithelium and having villi. FIG. 11 shows results of staining, with a smooth muscle actin antibody, an anti-PGP9.5 antibody and DAPI, of tissues formed by culture on the cell culture substrate having annular cell-adhesive parts having an inside diameter of 380 μm and a width of 60 μm in Example 1. The results shown in FIG. 11 show that the tissues formed in Example 1 had endoderm-derived intestinal epithelial tissues as well as mesoderm-derived smooth muscle actin-positive muscle tissues or ectoderm-derived PGP9.5-positive nerve fiber-like tissues. The results shown in FIG. 10 and FIG. 11 show that the tissues formed in Example 1 included tissues derived from 3 germ layers.

### Example 6

In order to study an appropriate size of an annular cell-adhesive part and the width of the adhesive part, the following analysis was conducted.

Each cell culture substrate having an annular cell-adhesive part having a different inside diameter and width was produced in the same manner as in Example 1. Edom iPS cells were cultured in the same manner as in Example 1. Tissues having a sac-shaped structure were classified by visual evaluation according to the state of formation into 3 grades: ++ (tissues having a sac-shaped structure were efficiently obtained), + (although differentiation into tissues having a sac-shaped structure occurred, many tissues were peeled off, or the generation of the tissues was as slow as that in Comparative Example 1), and − (no tissue was obtained because cells were peeled off or failed to cover by cell growth in the process of culture).

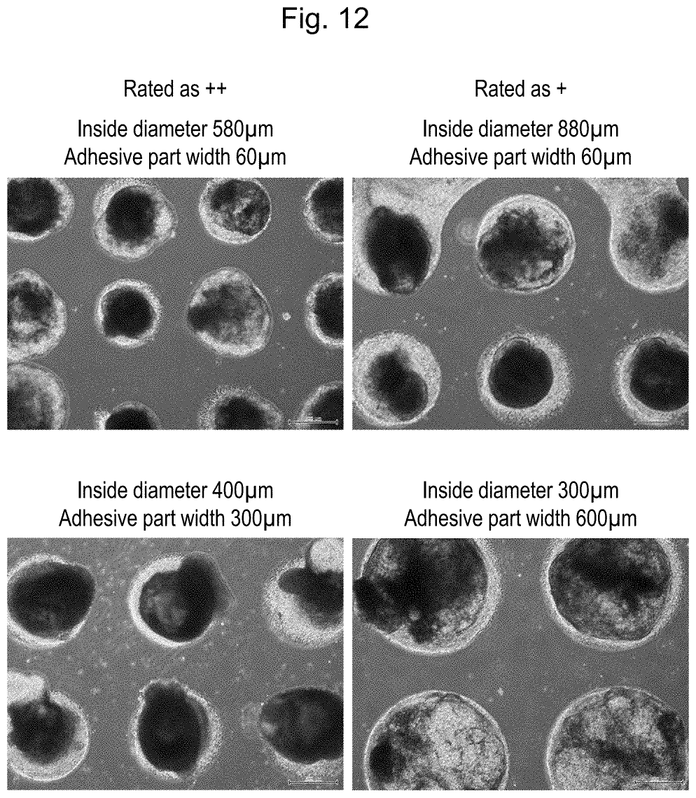

Observation images of typical examples are shown in FIG. 12. FIG. 12 is observation images on day 18 of culture on a cell culture substrate having an annular cell-adhesive part of each size. When rated “++” (left columns), tissues having a sac-shaped structure were detached and collected within 3 weeks.

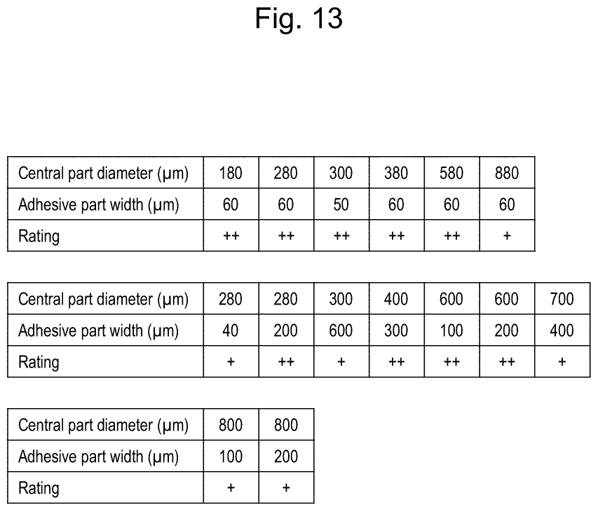

The results are shown in FIG. 13. FIG. 13 also includes the analysis results in Example 1. These results show that the inside diameter of the annular cell-adhesive part is preferably 180 μm to 880 μm and more preferably 180 μm to 600 μm, and show that the width of the annular cell-adhesive part is preferably in a range of 30 μm to 400 μm, more preferably in a range of 40 μm to 400 μm, and particularly preferably 60 μm to 300 μm.

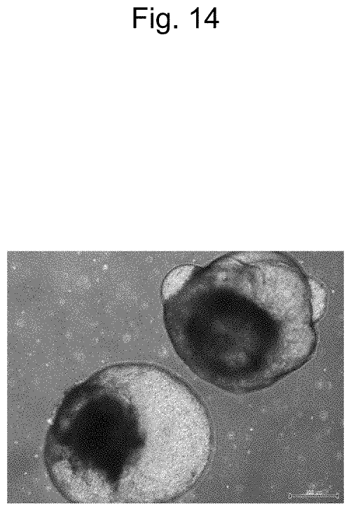

FIG. 14 shows an observation image of a tissue having a sac-shaped structure obtained by culture on a cell culture substrate having an annular cell-adhesive part having an inside diameter of 580 μm and a width of 60 μm.

### Example 7

The following study was conducted on the shape of a cell-adhesive part.

The studied shapes of cell-adhesive parts in cell culture substrates of Example were as shown in FIG. 15A, FIG. 15B, FIG. 15C, and FIG. 15D.
(15A) A square cell-adhesive part having an inside dimension of 280 μm to 300 μm in side and a width of 50 μm to 60 μm
(15B) An annular cell-adhesive part having an inside diameter of 280 μm and a width of 60 μm and lacking ⅛ in the circumferential direction
(15C) A rectangular cell-adhesive part having an inside dimension of 600 μm in long side and 300 μm in short side and a width of 50 μm
(15D) A square cell-adhesive part having an inside dimension of 600 μm in side and a width of 50 μm

Methods for producing these cell culture substrates and a cell culture method are as described in Example 1.

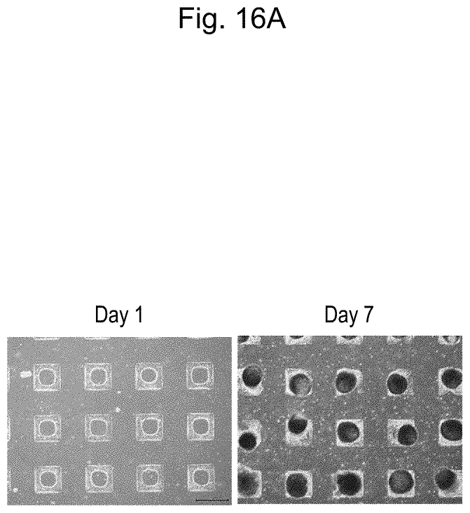

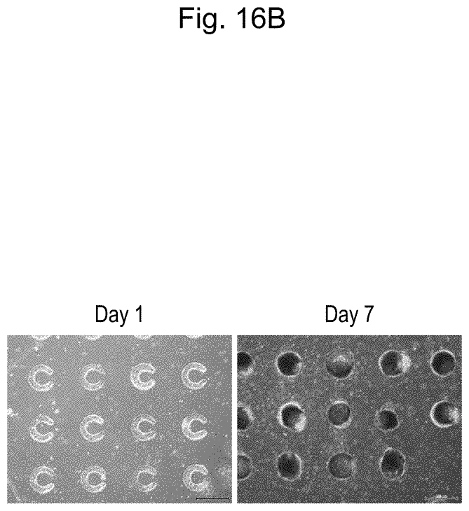

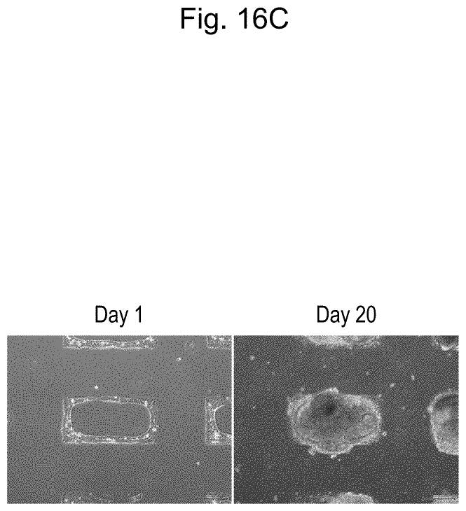

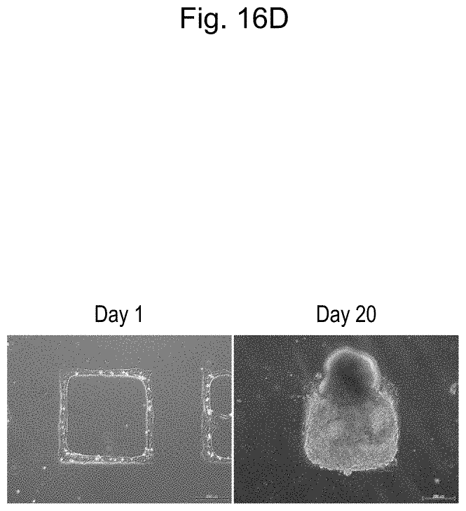

FIGS. 16A, 16B, 16C, and 16D show observation images of cultures obtained by culturing Edom iPS cells using cell culture substrates having cell-adhesive parts in the shapes shown in FIGS. 15A, 15B, 15C, and 15D, respectively. In the case of using the cell culture substrate having any of the shapes of the cell-adhesive parts, tissues having a sac-shaped structure were obtained in a short period, as in the other Examples. Even in the annular cell-adhesive part lacking ⅛ in the circumferential direction shown in FIG. 15B, tissues having a sac-shaped structure were obtained, as in the circumferentially completely annular cell-adhesive part, because the missing part was covered with cells that grew.

From these results, it is evident that the shape of the cell-adhesive part is not particularly limited and is not necessarily required to be round. Furthermore, a closed system can be created by culture, and the initial pattern is not necessarily required to be a closed pattern. Moreover, even a cell-adhesive part having a rectangular shape surrounding a non-cell-adhesive part also yielded a gut structure, suggesting that the non-cell-adhesive part surrounded by the cell-adhesive part does not have to have equal vertical and horizontal lengths and the non-cell-adhesive part surrounded by the cell-adhesive part may be oval or semicircular in shape.

### Example 8

In Example 1, the non-cell-adhesive part was formed by coating with polyethylene glycol. In this Example, whether other compounds used instead of polyethylene glycol could produce similar effects was studied. Accordingly, a cell culture substrate having a plurality of annular cell-adhesive parts having an inside diameter of 280 μm or 380 μm and a width of 60 μm was produced by the following method.

Glass (170 μm thick) was cut into 125 mm square as a substrate, immersed in an alkali washing solution PARKEM (Parker Corp., PK-LCG23) for 48 hours or longer for preliminary washing, and rinsed with pure water. Thereafter, the substrate was irradiated with vacuum ultraviolet ray (172 nm) for 6 minutes in a nitrogen atmosphere. Next, as a process of forming an annular pattern, a photosensitive dry film resist (Nichigo-Morton Co., Ltd., NIT915) was laminated onto the washed glass substrate on a hot plate of 100° C., and heated and kept for 5 minutes. Thereafter, the substrate was irradiated with 200 mJ of UV light (broad band) via a photomask having openings of the same size as that of annular patterns of the size described above. Resist patterns were formed by treatment with an aqueous sodium carbonate solution for 2 minutes, followed by baking at 100° C. for 5 minutes and subsequent step baking at 180° C. for 5 minutes. The substrate thus treated was cut into 15 mm×25 mm square. Aside from this, 0.5 wt % Lipidure(R) (NOF Corp.) was dissolved in 99.5% ethanol to prepare a solution, which was then used in an amount on the order of 200 μl to coat the substrate cut out by cast coating. The substrate was naturally dried for 1 day, then immersed in AZ Remover 100 (Tokyo Ohka Kogyo Co., Ltd.) under ultrasonic application for 5 minutes for resist removal, and then rinsed. Finally, EOG sterilization treatment was performed for 22 hours. Thus, a substrate having a plurality of annular cell-adhesive parts having an inside diameter of 280 μm or 380 μm and a width of 60 μm where the surface of the glass substrate was exposed, and non-cell-adhesive parts where the surface (inside and outside the annular cell-adhesive part) of the glass substrate was coated with Lipidure®, was obtained. The cell culture substrate in this Example has a cross-sectional structure as shown in FIG. 1(B), as in Example 1.

The substrate was cut into a size of 15 mm×25 mm and studied by seeding iPS cells, as in Example 1.

The film thicknesses of the Lipidure® coatings constituting the non-cell-adhesive parts on the substrate were measured in a profilometer and were consequently 288 nm on average.

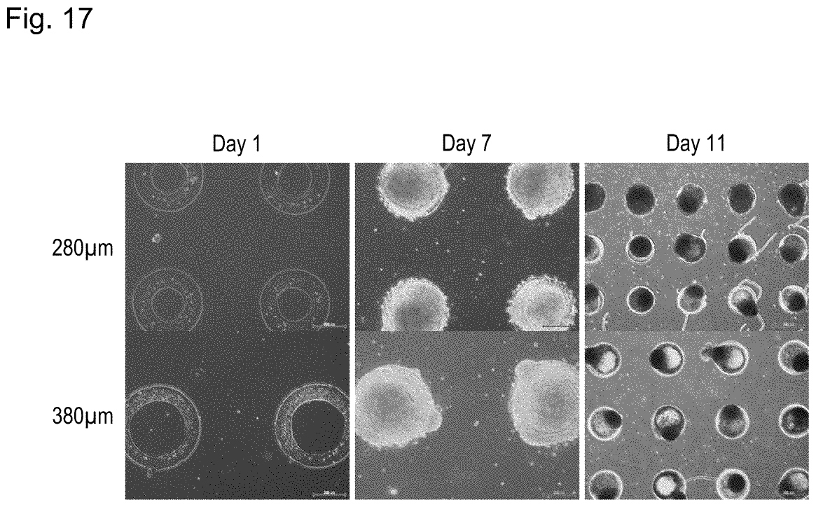

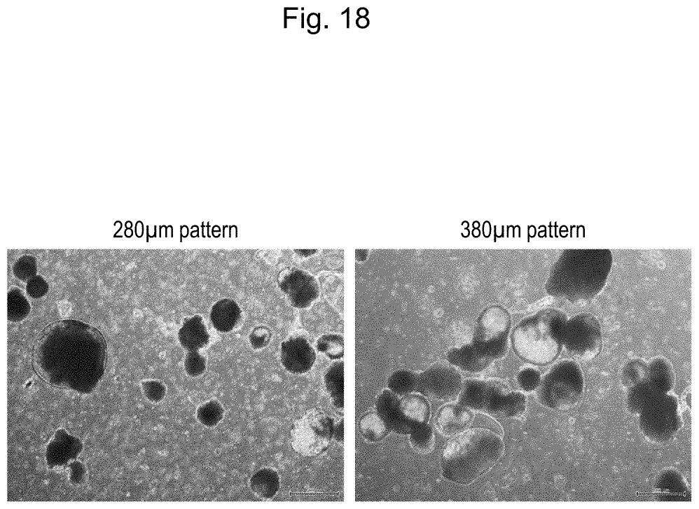

FIG. 17 shows observation images on culture days 1, 7 and 11 of a culture on each substrate. The magnification factors of the observation images on culture days 1 and 7 are higher than that of the observation image on culture day 11. FIG. 18 shows tissues having a sac-shaped structure obtained by culture for 3 weeks.

In Example 1 or other examples, the non-cell-adhesive parts were prepared by coating a substrate surface with polyethylene glycol, a compound suppressing cell adhesion. The results of this experiment show that in the case of using a substance other than polyethylene glycol as a compound suppressing cell adhesion, a non-cell-adhesive part can also be formed, as in polyethylene glycol.

### Example 9

Tissues obtained by cell culture on a cell culture substrate having an annular cell-adhesive part were analyzed for their features.

Cell culture substrates having an annular pattern having an inside diameter of 180 μm and a width of 60 μm, an annular pattern having an inside diameter of 280 μm and a width of 60 μm, an annular pattern having an inside diameter of 380 μm and a width of 60 μm, an annular pattern having an inside diameter of 600 μm and a width of 100 μm, an annular pattern having an inside diameter of 600 μm and a width of 200 μm, an annular pattern having an inside diameter of 800 μm and a width of 100 μm, or an annular pattern having an inside diameter of 800 μm and a width of 200 μm as a cell-adhesive part were produced by the procedures described in Example 1, and used as substrates in Example. By using these substrates, Edom iPS cells were cultured by the same procedures as in Example 1. After adhesion culture for 2 to 4 weeks, tissues that adhered onto the cell culture substrates were forcedly detached by flowing a medium. After the detachment, suspension culture was further performed for 1 to 3 weeks, and the resulting tissues were used in the following analysis.

For comparison, tissues obtained in Comparative Example 1 (using a cell culture substrate having a plurality of round cell-adhesive parts of 1500 μm in diameter) described above were similarly analyzed (as Comparative Example 1). For further comparison, by using a substrate produced by the procedures described in Example 1, which had an annular cell-adhesive part having an inside diameter of 600 μm and a width of 100 μm as a cell-adhesive part, Edom iPS cells were cultured by the same procedures as in Example 1, and tissues that adhered for 1 month or longer without being spontaneously detached were forcedly detached by flowing a medium. After the detachment, suspension culture was further performed for 1 to 2 weeks, and the resulting tissues were similarly analyzed (as Comparative Example 4).

The tissues obtained under each condition were washed with PBS. Thereafter, QIAzol Lysis Reagent (Qiagen N.V.) was added thereto in an amount of 900 μl, and then, the tissues were lysed with a homogenizer. Next, mRNA was extracted by operation using RNeasy Plus Universal Mini Kit (Qiagen N.V.) and chloroform (Nacalai Tesque, Inc.) according to the attached instruction manual, and suspended in RNase-free water. RNA concentration and purity were measured with Nanodrop. cDNA was produced by reverse transcription operation using RNA in an amount of 100 to 200 ng and SuperScript IV VILO Master Mix (Invitrogen Corp.), and then, the operation of real-time quantitative PCR (qPCR) was performed by the method described in Uchida et al., JCI Insight, Vol. 2, e86492, 2017. cDNA (BioChain Institute Inc.) derived from the human small intestine (this small intestine is a product that is not limited by a site as mentioned later) or duodenum, jejunum and ileum was used so as to be compared, and diluted in an amount of 1 μl in 200 μl of UltraPure Distilled Water (Invitrogen Corp.). Here, PCR Ready First Strand cDNA—Human Adult Normal Tissue: Small Intestine (BioChain Institute Inc.) was used as cDNA derived from the human small intestine that was not limited by a site. PCR Ready First Strand cDNA-Human Adult Normal Tissue: Ileum (BioChain Institute Inc.) was used as cDNA derived from the human ileum. After normalization based on the GAPDH levels, ABCB1 and ABCG2 levels in each tissue were determined when the values of ABCB1 and ABCG2 levels in the small intestine were each defined as 1. From the determined ABCB1 and ABCG2 levels, the ratio of the ABCG2 level to the ABCB1 level (ABCG2/ABCB1) was further determined. Likewise, after normalization based on the GAPDH levels, villin and CUBN levels in each tissue were determined when the values of villin and CUBN levels in the ileum having the expression of CUBN were each defined as 1. From the determined villin and CUBN levels, the ratio of the CUBN level to the villin level (CUBN/villin) was further determined.

The primers used here are as described below.

ABCB1 (forward primer)
(SEQ ID NO: 1)
5′-AAGGCCTAATGCCGAACACA-3′
ABCB1 (reverse primer)
(SEQ ID NO: 2)
5′-GTCTGGCCCTTCTTCACCTC-3′
ABCG2 (forward primer)
(SEQ ID NO: 3)
5′-GTTTATCCGTGGTGTGTCTGG-3′
ABCG2 (reverse primer)
(SEQ ID NO: 4)
5′-CTGAGCTATAGAGGCCTGGG-3′
villin (forward primer)
(SEQ ID NO: 5)
5′-CGGAAAGCACCCGTATGGAG-3′
villin (reverse primer)
(SEQ ID NO: 6)
5′-CGTCCACCACGCCTACATAG-3′
CUBN (forward primer)
(SEQ ID NO: 7)
5′-AATGGATGTGTGCAGCTCAG-3′
CUBN (reverse primer)
(SEQ ID NO: 8)
5′-GGGGTTGCTCAAACACTCAT-3′
GAPDH (forward primer)
(SEQ ID NO: 9)
5′-CCTGCACCACCAACTGCTTA-3′
GAPDH (reverse primer)
(SEQ ID NO: 10)
5′-GGCCATCCACAGTCTTCTGAG-3′

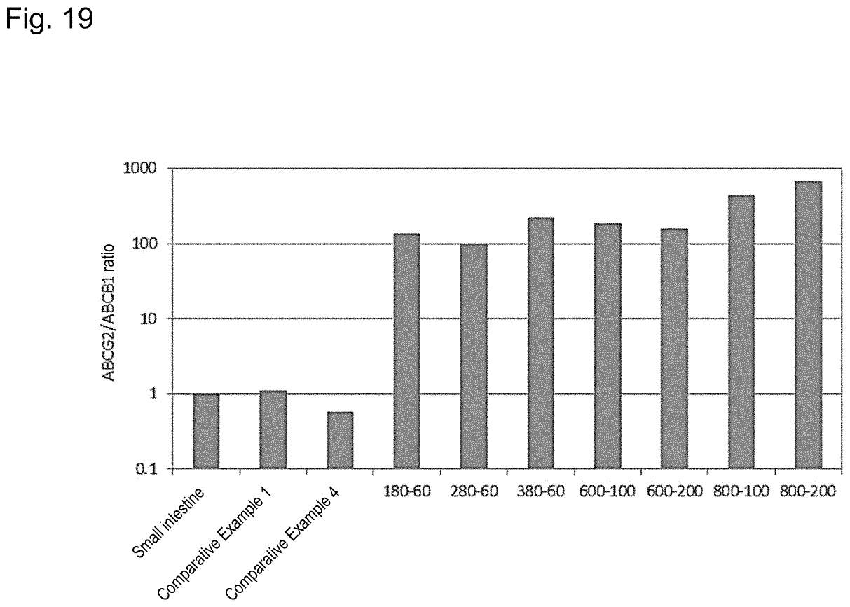

FIG. 19 shows an average ABCG2/ABCB1 ratio under each condition. All the notations in the graph, except for Comparative Example 1 and Comparative Example 4, mean the inside diameter (unit: μm)-width (unit: μm) of the annular cell-adhesive part. The range of the ABCG2/ABCB1 ratio in Comparative Example 1 was from 0.084 to 3.65, and the ABCG2/ABCB1 ratio in Comparative Example 4 was 0.44. From these results, tissues that were obtained according to the present disclosure and detached relatively early had a high expression ratio of ABCG2 to ABCB1, suggesting that tissues differing in properties from the gut organoid described in Uchida et al., JCI Insight, Vol. 2, e86492, 2017 can be obtained. It is considered that the range of the ABCG2/ABCB1 ratio is preferably larger than 90.

From the results of Comparative Example 4, a patterned substrate according to an embodiment of the present disclosure can also yield tissues having an ABCG2/ABCB1 ratio as low as that of the gut organoid described in Uchida et al., JCI Insight, Vol. 2, e86492, 2017, by long-term adhesion culture.

According to the description of Uchida et al., JCI Insight, Vol. 2, e86492, 2017, long-term culture for 30 days or longer in the pattern of Comparative Example 1 using human ES cells resulted in an ABCB1 transporter level higher than an ABCG2 transporter level, suggesting that the ABCG2/ABCB1 ratio is 1 or less.

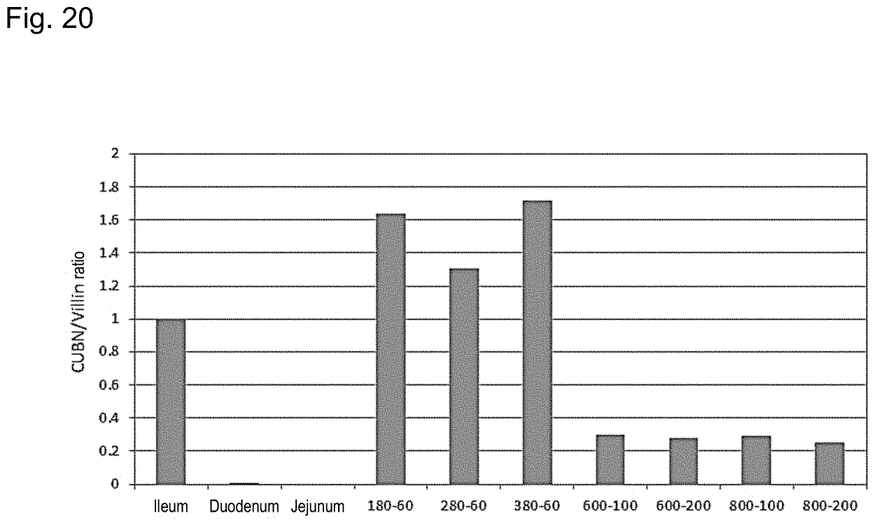

FIG. 20 shows average CUBN/villin measurement results. These results show that the range of the CUBN/villin ratio is preferably from 0.2 to 1.8. For each tissue having a CUBN/villin ratio that falls within the above range, the time of detachment from a cell culture substrate and the period of suspension culture are not constant, and tissues obtained by adhesion culture for 2 to 4 weeks and subsequent detachment, followed by further suspension culture for 1 to 3 weeks are included. This supports that tissues having a CUBN/villin ratio that fall within the above range are not limited to tissues having an adhesion culture period and a suspension culture period in particular ranges.

### Example 10

The following analysis was conducted on change in the properties of a portion having a high cell density (accumulating portion) in tissues.

Tissues obtained in Comparative Example 1 described above using a substrate having round cell-adhesive parts of 1500 μm in diameter were immunostained by the method described in Example 3. The primary antibodies used were a mouse IgG-labeled anti-Oct3/4 antibody for the undifferentiated cell marker (Santa Cruz Biotechnologies, Inc.; dilution ratio: 1/200) and a rabbit IgG-labeled anti-Ki67 antibody for the cell growth marker (Abcam plc; dilution ratio: 1/500).

The results are shown in FIG. 21. These results suggest that the expression of undifferentiated cell maekers was decreased in the portion having a high cell density (accumulating portion) in tissues.

Also, the following study was conducted on the presence or absence of difference depending on a container.

Edom iPS cells were seeded to a dish for cell culture having a diameter of 3.5 cm (culture dish), a glass substrate without any pattern (both, 1.5 cm×2.5 cm in size) (blank glass), or a substrate having a round cell-adhesive part having a diameter of 1500 μm (patterned substrate), and cultured in the same manner as in Example 1. In order to standardize a cell density, 5×10⁶ cells were seeded as to the dish for cell culture.

After culture for 7 days on each of the substrates, immunostaining was performed according to the method described in Example 4. The primary antibody used here was a mouse IgG-labeled anti-CDX2 antibody (BioGenex Laboratories; dilution ratio: 1/500), and the secondary antibody was an Alexa 546-labeled anti-mouse IgG antibody. The nuclei of the cells were stained with DAPI.

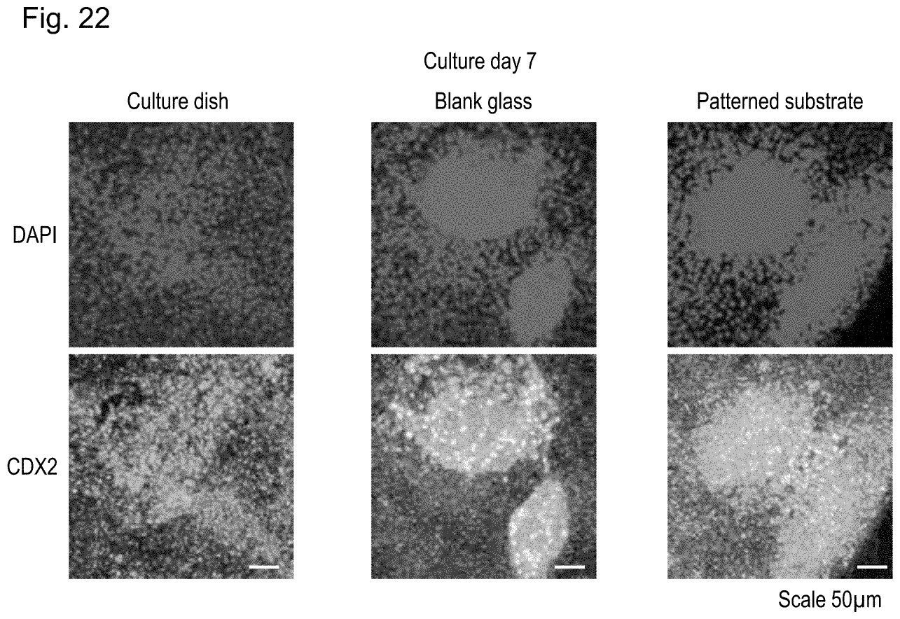

The results are shown in FIG. 22. These results suggest that differentiation into CDX2-positive trophectodermal cells take place in the portion having a high cell density (accumulating portion) in formed tissues, irrespective of the type of a substrate.

### Example 11

Genetic analysis was conducted in relation to the analysis of Example 9.

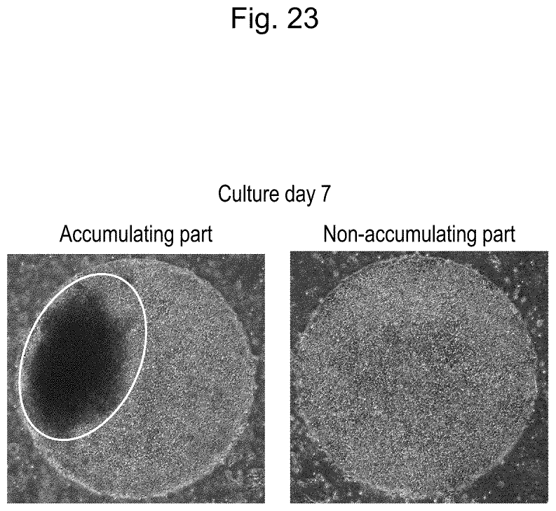

FIG. 23 shows observation images of tissues on culture day 7 when Edom iPS cells were cultured by the procedures of Example 1 on a substrate having a round cell-adhesive part having a diameter of 1500 μm in Comparative Example 1. The accumulating portion refers to the portion having dense accumulations of cells surrounded by the ellipse in the left photograph of FIG. 23 in a tissue that adhered to a cell-adhesive part. The non-accumulating portion refers to a portion without dense accumulations of cells as shown in the right photograph of FIG. 23 in a tissue that adhered to a cell-adhesive part (in the right photograph of FIG. 23, the whole tissue is a non-accumulating portion). Cells of the accumulating portion and cells of the non-accumulating portion from tissues on culture day 7 were separately sampled using a glass capillary. From each sample of the cells of the accumulating portion and the cells of the non-accumulating portion, RNA was obtained using RNeasy Plus Mini Kit (Qiagen N.V.), and suspended in RNase-free water. RNA concentration and purity were measured with NanoDrop™ (Thermo Fisher Scientific Inc.). cDNA was prepared by reverse transcription using RNA in an amount of 100 to 200 ng and SuperScript IV VILO Master Mix (Invitrogen Corp.), and then, the operation of real-time quantitative PCR (qPCR) was performed by the method described in Uchida et al., JCI Insight, Vol. 2, e86492, 2017 and Example 9.

The primers used here are as described below.

Oct3/4 (forward primer)
(SEQ ID NO: 11)
5′-CGAGGAATTTGCCAAGCTCTGA-3′
Oct3/4 (reverse primer)
(SEQ ID NO: 12)
5′-TTCGGGCACTGCAGGAACAAATTC-3′
GAPDH (forward primer)
5′-CCTGCACCACCAACTGCTTA-3′
(which is the same as SEQ ID NO: 9)
GAPDH (reverse primer)
5′-GGCCATCCACAGTCTTCTGAG-3′
(which is the same as SEQ ID NO: 10)

For each sample of the accumulating portion and the non-accumulating portion, the expression level of Oct3/4 was normalized with the expression level of GAPDH to determine a normalized value. Then, the relative value of the expression level of Oct3/4 in the sample of the accumulating portion was determined when the expression level of Oct3/4 in the sample of the non-accumulating portion was defined as 1.

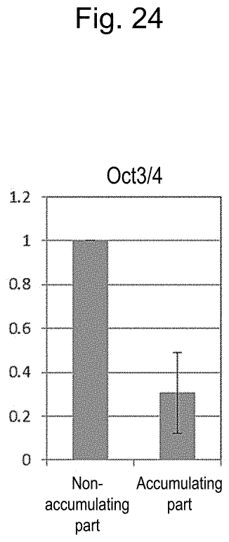

The results are shown in the graph of FIG. 24. These results suggest that the expression of the undifferentiated cell marker Oct3/4 gene was significantly decreased in the accumulating portion with respect to the non-accumulating portion. From these results, when pluripotent stem cells were cultured on a substrate having a cell-adhesive part, the expression of the undifferentiated cell marker Oct3/4 gene was decreased in an accumulating portion formed by the dense accumulations and adhesion of cells in the cell-adhesive part, indicating that trophectodermal cells are likely to be produced.

The characteristics of the accumulating portion of a culture of pluripotent stem cells observed in Examples 10 and 11 are characteristics common in accumulating portions formed by dense accumulations of pluripotent stem cells during culture. Therefore, it is considered that an accumulating portion formed by dense accumulations of pluripotent stem cells in an annular cell-adhesive part also has similar characteristics.

### Example 12

In order to examine the properties of epithelial cells in an obtained gut organoid, the following analysis was conducted.

Edom iPS cells were cultured in the same manner as in Example 1 on a cell culture substrate having a plurality of annular cell-adhesive parts having an inside diameter of 580 μm and a width of 60 μm produced in the same manner as in Example 1, and a cell culture substrate having a plurality of round cell-adhesive parts of 1500 μm in diameter produced in Comparative Example 1, to produce sac-shaped tissues (gut organoids). By using the obtained tissues, immunostaining was performed by the same method as described in Example 5 described above. As antibodies, a mouse IgG1-labeled anti-cytokeratin 7 antibody (KRT7; Abcam plc, dilution ratio: 1/500) and a rabbit IgG-labeled anti-CDX2 antibody used in Example 4 described above were used. The same secondary antibodies as in Example 4 were also used, and the nuclei of the cells were stained with DAPI.

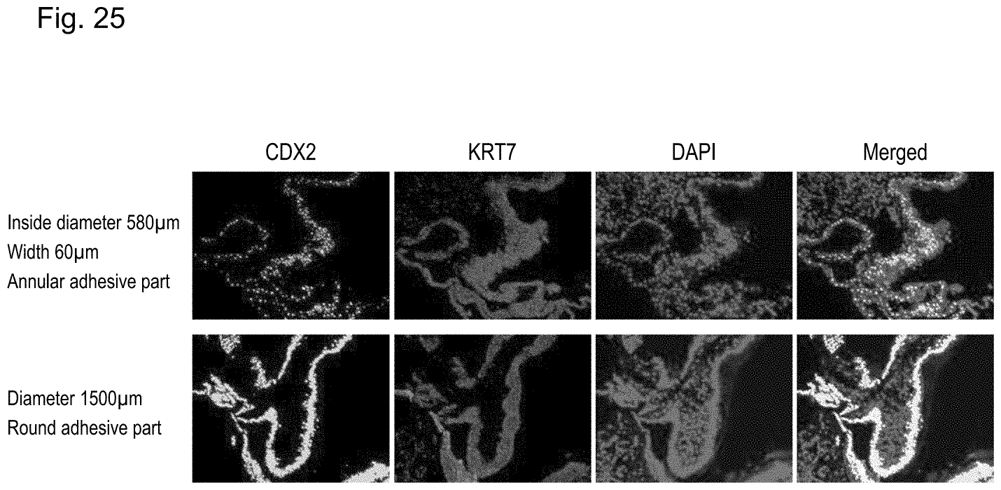

The results are shown in FIG. 25. From FIG. 25, in sac-shaped tissues, CDX2-positive cells of epithelial cells also expressed KRT7. It was further suggested that this property did not depend on a pattern shape and was derived from CDX2-positive trophectodermal cells found in an accumulating portion found inside the pattern.

### Example 13

In order to study whether nerve cells would be migrated from an accumulating portion of cells formed during culture on a cell culture substrate having a pattern of a cell-adhesive part, the following analysis was conducted.

Edom iPS cells were cultured under the same conditions as in Example 1 and other examples. except that as a cell culture substrate, a cell culture substrate having a plurality of round cell-adhesive parts of 1500 μm in diameter used in Comparative Example 1 was used in consideration of easy operation of collection and easy distinguishment between an accumulating portion and a non-accumulating portion.

One week and two weeks after the start of culture, the culture was observed under a stereo microscope, and the formed accumulating portion of cells was collected with a glass capillary and re-seeded in the state of masses having an appropriate number on a vitronectin-coated culture dish of 3.5 cm in diameter (AGC Inc.). As a medium, the XF hESC culture medium described in Non Patent Literature 3 was used, as in Example 1. Then, the culture was maintained for several days, followed by the identification of nerve cells by immunostaining.

The staining method was the same as in Example 4. The primary antibodies used were a mouse IgG1-labeled anti-nestin antibody (Sigma-Aldrich Co. LLC, dilution ratio: 1/500) and a rabbit IgG-labeled anti-βIII tubulin antibody (Abcam plc, dilution ratio: 1/1000).

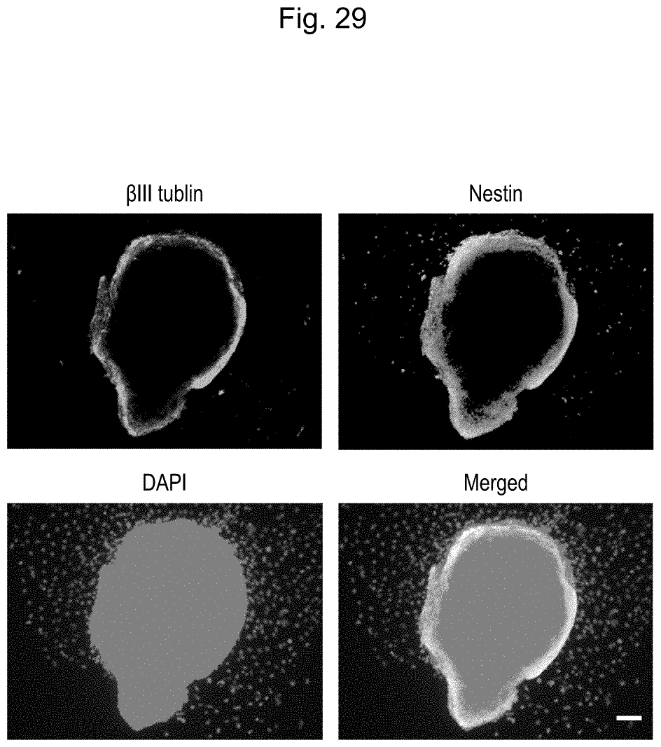

FIG. 29 shows results of immunostaining a culture obtained by re-culturing for 4 days the accumulating portion collected 1 week after the start of culture. The presence of nerve marker-positive cells was found in the outer circumference of the accumulating portion. In other words, it is considered that the development of nerve cells occurred from the accumulating portion.

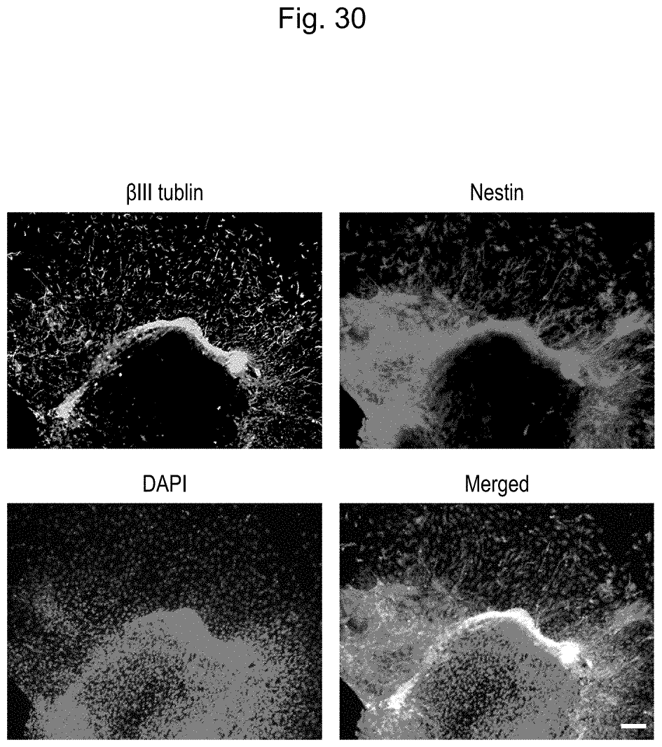

FIG. 30 shows results of immunostaining a culture obtained by re-culturing for 1 day the accumulating portion collected 2 weeks after the start of culture. It was suggested that nerve cells having a high migratory capacity were contained in the accumulating portion at this point of time. It is predicted that nerve cells having a high migratory capacity are capable of spreading and covering a non-cell-adhesive part (central part).

### Example 14

In order to find conditions under which cells and a sac-shaped cell construct obtained on a cell culture substrate were maintained while adhering on the substrate without being detached, the following study was conducted.

By using a cell culture substrate having a plurality of annular cell-adhesive parts having an inside diameter of 600 μm and a width of 100 μm produced by the method described in Example 1, Edom iPS cells were cultured under the same conditions as in Example 1.

On day 11 from the start of culture, the medium was replaced with each of the XF hESC medium (control; the same as that used until day 11) described in Non Patent Literature 3, Hank's buffer solution (HBSS(+), Nacalai Tesque, Inc.) containing calcium ions and magnesium ions, a medium obtained by removing the nutrient factors bFGF, heregulin, and IGF-1 from the XF hESC medium (GF(−) medium), and a medium obtained by removing the nutrient factors from the XF hESC medium and further adjusting an XF-KSR (Knockout Serum Replacement XF CTS (XF-KSR; Life Technologies Corp.)) concentration to 1% (1% XF-KSR medium). The culture was continued, and photographs were obtained by appropriate observation.

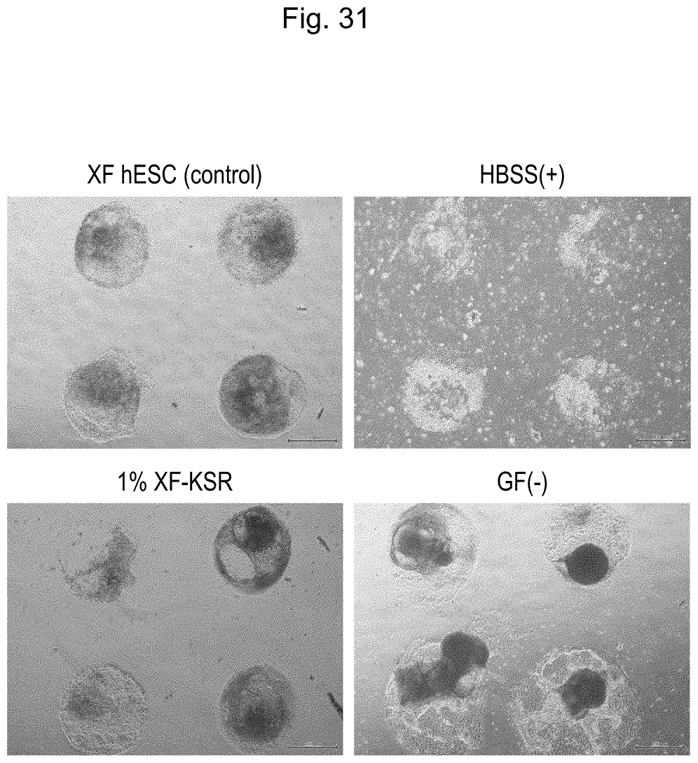

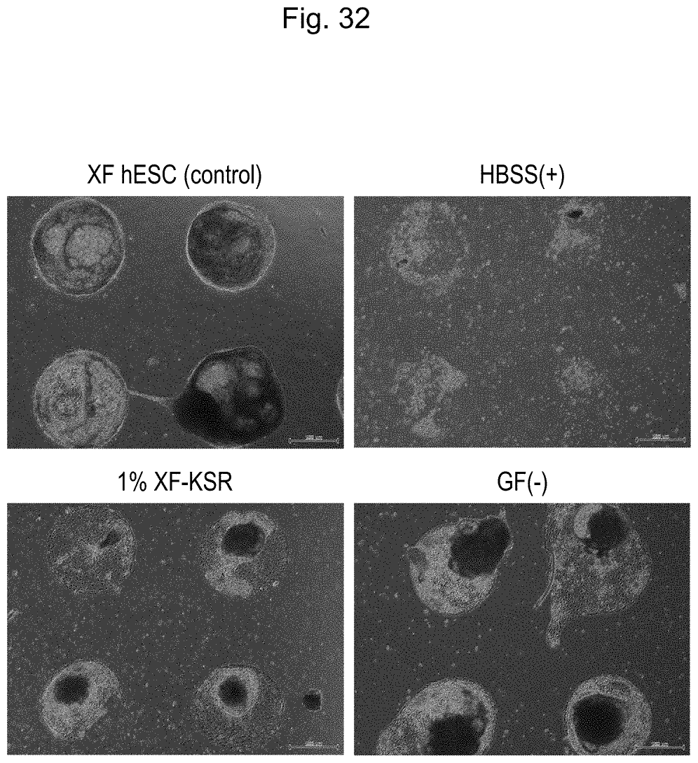

FIG. 31 shows observation images of cultures obtained by culture for 4 days after medium replacement. FIG. 32 shows observation images of cultures obtained by culture for 10 days after medium replacement. It was confirmed that cell detachment was observed in HBSS(+) which was thus unsuitable, whereas small sac-shaped structures and cells were maintained in the media free from the nutrient factors (GF(−) and 1% XF-KSR) while adhering on the cell culture part of the cell culture substrate. In the control, sac-shaped structures having an ordinary size and shape were observed at the stage of FIG. 32, which was 3 weeks after the start of culture. Thus, it is considered that the culture itself was normal.

When the culture was further continued, spontaneous detachment of the sac-shaped cell constructs and the accompanying cells were observed in the control, whereas the maintenance of adhesion of the cells and the sac-shaped cell constructs was observed in the media free from the nutrient factors (GF(−) and 1% XF-KSR). This suggests that: a sac-shaped cell construct can be maintained in a medium free from nutrient factors while adhering on a cell culture substrate; and cells or tissues in the cell construct are maintained.

All publications, patents and patent applications cited herein are incorporated herein by reference in their entirety.

1.-13. (canceled)

14. A method for producing a cell construct comprising small intestinal epithelial cells, comprising:
seeding stem cells onto a cell culture substrate,
the cell culture substrate having a surface comprising a cell culture part, the cell culture part comprising a non-cell-adhesive part, and a cell-adhesive part extending continuously or intermittently along the periphery of the non-cell-adhesive part and surrounding the non-cell-adhesive part; and
culturing the stem cells seeded to differentiate a part of the stem cells into small intestinal epithelial cells.

15. The method according to claim 14, wherein
the cell culture substrate comprises a support substrate having a surface comprising
a first non-cell-adhesive part, and
the one or more cell culture parts arranged in the first non-cell-adhesive part, wherein
each of the one or more cell culture parts comprises a central part serving as a second non-cell-adhesive part, and the cell-adhesive part extending continuously or intermittently along the periphery of the central part and surrounding the central part, wherein a cell-adhesive part is not present in the second non-cell-adhesive part.

16. The method according to claim 14, wherein a distance between two points of intersection of a straight line with an inner circumference of the cell-adhesive part is larger than 80 μm and 880 μm or smaller, the straight line passing through a midpoint between two points on the inner circumference of the cell-adhesive part opposed to and most distant from each other across the non-cell-adhesive part.

17. The method according to claim 14, wherein a width of the cell-adhesive part along a straight line is larger than 30 μm and 400 μm or smaller, the straight line passing through a midpoint between two points on an inner circumference of the cell-adhesive part opposed to and most distant from each other across the non-cell-adhesive part.

18. The method according to claim 14, wherein
the cell construct comprises small intestinal epithelial cells, wherein
when an expression level of ABCG2 transporter and an expression level of ABCB1 transporter in the cell construct are indicated as relative values with respect to an expression level of ABCG2 transporter and an expression level of ABCB1 transporter, respectively, in a small intestinal tissue of an origin animal, the relative value of the expression level of ABCG2 transporter in the cell construct is 90 or more times the relative value of the expression level of ABCB1 transporter.

19. The method according to claim 18, wherein each of the expression levels is based on a mRNA level.

20. The method according to claim 18, wherein the cell construct comprises endodermal cells, ectodermal cells and mesodermal cells.

21. A cell construct comprising small intestinal epithelial cells, wherein
when an expression level of ABCG2 transporter and an expression level of ABCB1 transporter in the cell construct are indicated as relative values with respect to an expression level of ABCG2 transporter and an expression level of ABCB1 transporter, respectively, in a small intestinal tissue of an origin animal, the relative value of the expression level of ABCG2 transporter in the cell construct is 90 or more times the relative value of the expression level of ABCB1 transporter.

22. The cell construct according to claim 21, wherein each of the expression levels is based on a mRNA level.

23. The cell construct according to claim 21, comprising endodermal cells, ectodermal cells and mesodermal cells.

24. A cell construct-holding substrate comprising:
the cell construct according to claim 21.

25. A test kit comprising:
the cell construct according to claim 21.

26. A test method comprising:
using the cell construct according to claim 21.

27. A cell construct-holding substrate comprising:
a cell culture substrate having a surface comprising a cell culture part, the cell culture part comprising a non-cell-adhesive part, and a cell-adhesive part extending continuously or intermittently along the periphery of the non-cell-adhesive part and surrounding the non-cell-adhesive part; and
a cell construct comprising small intestinal epithelial cells adhering onto the cell culture part.

28. The cell construct-holding substrate according to claim 27, wherein
the cell culture substrate comprises a support substrate having a surface comprising
a first non-cell-adhesive part, and
the one or more cell culture parts arranged in the first non-cell-adhesive part, wherein
each of the one or more cell culture parts comprises a central part serving as a second non-cell-adhesive part, and the cell-adhesive part extending continuously or intermittently along the periphery of the central part and surrounding the central part, wherein a cell-adhesive part is not present in the second non-cell-adhesive part.

29. The cell construct-holding substrate according to claim 27, wherein when the cell construct-holding substrate is viewed along the normal direction of the surface, the small intestinal epithelial cells are present at least at a position overlapping the cell-adhesive part.

30. The cell construct-holding substrate according to claim 27, wherein
when the cell construct-holding substrate is viewed along the normal direction of the surface, the cell construct overlaps the cell-adhesive part and the non-cell-adhesive part, and
the small intestinal epithelial cells are present in a larger amount at a position overlapping the cell-adhesive part than at a position overlapping the non-cell-adhesive part.

31. The cell construct-holding substrate according to claim 27, wherein the cell construct comprises endodermal cells, ectodermal cells and mesodermal cells.

32. The cell construct-holding substrate according to claim 27, wherein a distance between two points of intersection of a straight line with an inner circumference of the cell-adhesive part is larger than 80 μm and 880 μm or smaller, the straight line passing through a midpoint between two points on the inner circumference of the cell-adhesive part opposed to and most distant from each other across the non-cell-adhesive part.

33. The cell construct-holding substrate according to claim 27, wherein a width of the cell-adhesive part along a straight line is larger than 30 μm and 400 μm or smaller, the straight line passing through a midpoint between two points on an inner circumference of the cell-adhesive part opposed to and most distant from each other across the non-cell-adhesive part.
